SEQUENCE LISTING (2 sequences)

SEQ ID NO: 1 - protein, 4 aa, synthetic construct
AEDG

SEQ ID NO: 2 - protein, 7 aa, synthetic construct
TKPRPGP

(19) United States
(12) Patent Application Publication
(10) Pub. No.: US 2025/0281531 A1
(43) Pub. Date: Sep. 11, 2025

(54) METHODS AND COMPOSITIONS FOR DETOXIFICATION OF CHRONIC EXPOSURE TO TOXINS

(71) Applicant: MDLifespan Enterprises, LLC, Branch, MI (US)

(72) Inventors: Paul Eldon Savage, Branch, MI (US); Edwin Lee, Orlando, FL (US)

(21) Appl. No.: 19/073,986

(22) Filed: Mar. 7, 2025

Related U.S. Application Data
(60) Provisional application No. 63/563,454, filed on Mar. 10, 2024.
(60) Provisional application No. 63/692,682, filed on Sep. 9, 2024.
(60) Provisional application No. 63/705,022, filed on Oct. 8, 2024.
(60) Provisional application No. 63/741,400, filed on Jan. 2, 2025.

(51) Int. Cl. A61K 35/16; A61K 9/00; A61K 31/194; A61K 31/198; A61K 31/4184; A61K 31/4196; A61K 31/5415; A61K 31/7048; A61K 31/727; A61K 33/38; A61K 35/28; A61K 35/51; A61K 38/06; A61K 38/07; A61K 38/08; A61K 38/17; A61K 38/22; A61K 38/38; A61K 39/395; A61K 45/06; A61P 39/00

(52) U.S. Cl. CPC A61K 35/16; A61K 9/0053; A61K 31/194; A61K 31/198; A61K 31/4184; A61K 31/4196; A61K 31/5415; A61K 31/7048; A61K 31/727; A61K 33/38; A61K 35/28; A61K 35/51; A61K 38/063; A61K 38/07; A61K 38/08; A61K 38/1709; A61K 38/2292; A61K 38/38; A61K 39/39516; A61K 45/06; A61P 39/00

(57) ABSTRACT

The disclosure relates to methods, compositions, and combinations for detoxification in a patient comprising performing therapeutic plasma exchange (TPE) on the patient. The disclosure also relates to methods, compositions, and combinations for removing toxins in a patient as a result of chronic exposure to the toxins, comprising performing one or more TPE sessions on the patient and administering an oral composition to the patient. The methods, compositions, and combinations of the disclosure may also be used to replenish biomarkers indicative of a healthy state of the body in order to improve immune system function or patient longevity, and/or reduce oxidative stress or inflammation. The methods, compositions, and combinations of the disclosure may also be used to reduce the level of atherosclerosis, improve cognitive function, prevent early cancer development, promote immune health, and/or treat health conditions or diseases such as autoimmune diseases, cancers, Alzheimer's disease, Parkinson's disease, chronic fatigue syndromes, or fibromyalgia. Additionally, the methods, compositions, and combinations of the disclosure may comprise an intravenous supplementation, wherein each TPE session is followed by administration to the patient of an IV composition. The disclosure further relates to oral compositions and IV compositions for use in the methods, compositions, and combinations of the disclosure.

### CROSS-REFERENCE TO RELATED APPLICATIONS

This application claims priority to U.S. Provisional Application Nos. 63/563,454, filed Mar. 10, 2024; 63/692,682, filed Sep. 9, 2024; 63/705,022, filed Oct. 8, 2024; and 63/741,400, filed Jan. 2, 2025, all of which are incorporated by reference herein in their entirety.

### REFERENCE TO AN ELECTRONIC SEQUENCE LISTING

The instant application contains a Sequence Listing that has been submitted electronically and is hereby incorporated by reference in its entirety. The Sequence Listing was created on Oct. 8, 2024, is named “24-0150-US-PRO3_Sequence_Listing.xml”, and is 2,572 bytes in size.

### FIELD OF THE DISCLOSURE

The disclosure relates to methods, compositions, and combinations for detoxification in a patient comprising performing therapeutic plasma exchange (TPE) on the patient. The disclosure also relates to methods, compositions, and combinations for removing toxins in a patient as a result of chronic exposure to the toxins, comprising performing one or more TPE sessions on the patient and administering an oral composition to the patient. The methods, compositions, and combinations of the disclosure may also be used to replenish biomarkers indicative of a healthy state of the body in order to improve immune system function or patient longevity, and/or reduce oxidative stress or inflammation. The methods, compositions, and combinations of the disclosure may also be used to reduce the level of atherosclerosis, improve cognitive function, prevent early cancer development, promote immune health, and/or treat health conditions or diseases such as autoimmune diseases, cancers, Alzheimer's disease, Parkinson's disease, chronic fatigue syndromes, or fibromyalgia. Additionally, the methods, compositions, and combinations of the disclosure may comprise an intravenous supplementation, wherein each TPE session is followed by administration to the patient of an IV composition. The disclosure further relates to oral compositions and IV compositions for use in the methods, compositions, and combinations of the disclosure.

### BACKGROUND

Current therapeutic options to remove toxins in the body are limited and generally are used only in emergency situations in which patients have been acutely exposed to the toxins at a dangerous level. For chemical toxins, aside from standard of care emergency medical response to acute toxicity with organophosphate (Robb et al., “Organophosphate Toxicity” in StatPearls [Internet](Treasure Island (FL), StatPearls Publishing, 2024), no approved or even experimental therapeutic procedures are available. Mitigation strategies with phytochemicals and antioxidants are suggested, but unfortunately these only address the downstream effects of chemical toxins (Sokan-Adeaga et al., J. Public Health Res. 12(2): 22799036231181226 (2023); Abdel-Daim et al., Oxid. Med. Cell. Longev. 2018: 6276438 (2018)). For heavy metals, treatment is limited to emergency medical intervention in which the patient is removed from toxic exposure. The heavy metals may be removed from the body by gastric lavage, activated charcoal, and skin decontamination. Supportive care can be in the form of intravenous fluids, oxygen, and ventilatory and circulatory support as needed. In severe cases, hemodialysis, plasma exchange, and extracorporeal membrane oxygenation (ECMO) may be necessary (Rajkumar et al., “Heavy Metal Toxicity,” in StatPearls [Internet](Treasure Island (FL), StatPearls Publishing, 2024). Metal chelation therapy, where a drug or drugs with binding affinity to metals are given to a patient constitutes the current standard of care for both acute and chronic exposure. Limitations of chelation therapy can include low efficacy, required co-treatments to improve efficacy and safety, and few drug options. The properties of an ideal chelating agent are high solubility, high cell permeability, the ability to bind most toxic heavy metals and form non-toxic compounds, sufficient efficacy by oral and parenteral routes, and a high rate of elimination. However, such ideal chelators have not yet been identified (Rajkumar et al., “Heavy Metal Toxicity,” in StatPearls [Internet](Treasure Island (FL), StatPearls Publishing, 2024; Sears, Scientific World Journal 2013: 219840 (2013)). While methods such as plasmapheresis or plasma exchange can be used for detoxification, such methods are known to be effective only for certain toxins and in cases of acute intoxication (Nenov et al., Nephrol. Dial. Transplant. 18(Suppl5): v56-v58 (2003); Yilmaz et al., Am. J. Emerg. Med. 31(6): 953-57 (2013)).

Thus, there remains a need for effective and safe methods of detoxification for both long-term and slow exposure to a wide variety of toxins.

### SUMMARY

The disclosure provides methods, compositions, and combinations for detoxification in a patient comprising performing therapeutic plasma exchange (TPE) on the patient. The disclosure also provides methods, compositions, and combinations for removing toxins in a patient as a result of chronic exposure to the toxins, comprising performing one or more TPE sessions on the patient and administering an oral composition to the patient. The methods, compositions, and combinations of the disclosure may also be used to replenish biomarkers indicative of a healthy state in order to improve immune system function or patient longevity, and/or reduce oxidative stress or inflammation. The methods, compositions, and combinations of the disclosure may also be used to reduce the level of atherosclerosis, improve cognitive function, prevent early cancer development, promote immune health, and/or treat health conditions or diseases such as autoimmune diseases, cancers, Alzheimer's disease, Parkinson's disease, chronic fatigue syndromes, or fibromyalgia.

The disclosure also provides methods, compositions, and combinations wherein plasma is removed from the patient at least once every 1 to 20 weeks. The disclosure further provides methods, compositions, and combinations wherein the step of removing plasma from the patient at least once every 1 to 20 weeks after a TPE session is performed by donating plasma.

The disclosure also provides methods, compositions, and combinations wherein each TPE session is accompanied by IV or subcutaneous infusion of immunoglobulin. The disclosure further provides methods, compositions, and combinations wherein a metal chelator is administered to the patient either by IV or orally throughout the TPE session.

The disclosure also provides methods, compositions, and combinations comprising additional supplementation, wherein each TPE session is followed by administration to the patient of an IV composition. The disclosure further provides oral compositions and IV compositions for use in the methods, compositions, and combinations of the disclosure.

The methods, compositions, and combinations of the disclosure may also be used to reduce the level of atherosclerosis in a patient. In such methods, compositions, and combinations, at least one cholesterol-reducing agent is administered to the patient after the first TPE session. In some methods, compositions, and combinations of the disclosure, the at least one cholesterol-reducing agent is an agent that has not been administered to the patient before the method is commenced on the patient. In some methods, compositions, and combinations of the disclosure, at least one regenerative material is administered to the patient.

The methods, compositions, and combinations of the disclosure may also be used to improve cognitive function in a patient with cognitive decline. In some methods, compositions, and combinations of the disclosure, low dose naltrexone is administered to the patient. In some methods, compositions, and combinations of the disclosure, at least one neuropeptide is administered to the patient. In some methods, compositions, and combinations of the disclosure, at least one regenerative material is administered to the patient.

The methods, compositions, and combinations of the disclosure may also be used to improve immune health in a patient. In some methods, compositions, and combinations of the disclosure, low dose naltrexone is administered to the patient. In some methods, compositions, and combinations of the disclosure, at least one regenerative material is administered to the patient.

The methods, compositions, and combinations of the disclosure may also be used to prevent early cancer development in patient. In some methods, compositions, and combinations of the disclosure, low dose naltrexone is administered to the patient. In some methods, compositions, and combinations of the disclosure, at least one anti-inflammatory compound, one anti-fungal compound, or a combination thereof is administered to the patient.

The disclosure also provides an IV composition for use in the methods, compositions, and combinations of the disclosure, wherein the IV composition comprises one or more of at least one mineral, at least one vitamin, and at least one amino acid.

The disclosure further provides an oral composition for use in the methods, compositions, and combinations of the disclosure, wherein the oral composition comprises one or more of at least one vitamin, at least one mineral, at least one anti-inflammatory agent, at least one antioxidant, at least one amino acid, at least one saccharide, and at least one herb. The oral composition may further comprise at least one protein. The oral composition may further comprise at least one other component. In some methods, compositions, and combinations of the disclosure, the oral composition is administered to the patient daily after the first TPE session.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTIONS OF THE DRAWINGS

FIG. 1 shows graphs depicting changes in the level of the indicated biomarkers of immune system function over time before and after combined TPE (with supplementation) treatment. CD=cluster of differentiation. Data shown as average of tested patients.

FIG. 2 shows graphs depicting changes in the level of the indicated biomarkers of longevity over time before and after combined TPE (with supplementation) treatment. NAD intracellular=nicotinamide adenine dinucleotide intracellular; β-gal=β-galactosidase; JPM U=Jinfiniti Precision Medicine Unit. Data shown as average of tested patients.

FIG. 3 shows graphs depicting changes in the level of the indicated biomarkers of inflammation over time before and after combined TPE (with supplementation) treatment. Hs-CRP=high-sensitivity C-reactive protein; TNF-α=tumor necrosis factor α; IL=interleukin. Data shown as average of tested patients.

FIG. 4 shows graphs depicting changes in the level of the indicated biomarkers of oxidative stress over time before and after combined TPE treatment (with supplementation). 8-OHdG=8-Oxoguanine; MPO=myeloperoxidase. Data shown as average of tested patients.

FIG. 5 shows graphs depicting changes in the level of the indicated heavy metals over time before and after combined TPE treatment (with supplementation). Data shown as average of tested patients.

FIG. 6 shows graphs depicting changes in the level of the indicated herbicides over time before and after combined TPE treatment (with supplementation). Data shown as average of tested patients.

FIG. 7 shows graphs depicting changes in the level of the indicated pesticides over time before and after combined TPE treatment (with supplementation). Data shown as average of tested patients.

FIG. 8 shows graphs depicting changes in the level of the indicated phenols over time before and after combined TPE treatment (with supplementation). Data shown as average of tested patients.

FIG. 9 shows graphs depicting changes in the level of the indicated phthalates over time before and after combined TPE treatment (with supplementation). Data shown as average of tested patients.

FIG. 10 shows graphs depicting changes in the level of the indicated volatile organic compounds over time before and after combined TPE treatment (with supplementation). 4MHA=4-methylhippuric acid; 2HIB=2-hydroxyisobutyric acid; PGO=phenylglyoxylic acid; NAE=N-acetyl-S-(2-carbamoylethyl)-cysteine. Data shown as average of tested patients.

FIG. 11 shows graphs depicting changes in the level of the indicated mycotoxins over time before and after combined TPE treatment (with supplementation). GenX/HPFO-DA=GenX or hexafluoropropylene oxide dimer acid; PFHpS=perfluoro-1-heptane sulfonic acid; PFDoA=perfluorododecanoic acid; PFHxS=perfluorohexane sulfonic acid; PFOS=perfluorooctane sulfonic acid. Data shown as average of tested patients.

FIG. 12 shows graphs depicting changes in the level of the indicated per- and polyfluorinated substances (PFAS) over time before and after combined TPE treatment (with supplementation). Data shown as average of tested patients.

FIG. 13 shows a graph depicting changes in the level of microplastic particles (MP) over time before and after combined TPE treatment (with supplementation). Data shown as average of tested patients.

FIG. 14 shows graphs depicting changes in the levels of the indicated biomarkers of immune system before (initial average) and 6 weeks after combined TPE, brain TPE, and cancer TPE treatments (final average). CD=cluster of differentiation. Data shown as average of tested patients. The shaded box in each graph indicates the healthy range.

FIG. 15 shows a graph depicting changes in the level of the indicated biomarker of oxidative stress before (initial average) and 6 weeks after combined TPE, brain TPE, and cancer TPE treatments (final average). MPO=myeloperoxidase. Data shown as average of tested patients. The shaded box in the graph indicates the healthy range.

FIG. 16 shows graphs depicting changes in the levels of the indicated biomarkers of inflammation before (initial average) and 6 weeks after combined TPE, brain TPE, and cancer TPE treatments (final average). Hs-CRP=high-sensitivity C-reactive protein; TNF-α=tumor necrosis factor α; IL=interleukin. Data shown as average of tested patients. The shaded box in each graph indicates the healthy range.

FIG. 17 shows graphs depicting changes in the levels of the indicated heavy metals before (initial average) and 6 weeks after combined TPE, brain TPE, and cancer TPE treatments (final average). Data shown as average of tested patients. The shaded box in each graph indicates the healthy range.

FIG. 18 shows graphs depicting changes in the levels of the indicated herbicides before (initial average) and 6 weeks after combined TPE, brain TPE, and cancer TPE treatments (final average). Data shown as average of tested patients. The shaded box in each graph indicates the healthy range.

FIG. 19 shows graphs depicting changes in the levels of the indicated pesticides before (initial average) and 6 weeks after combined TPE, brain TPE, and cancer TPE treatments (final average). Data shown as average of tested patients. The shaded box in each graph indicates the healthy range.

FIG. 20 shows a graph depicting changes in the level of the indicated phenol before (initial average) and 6 weeks after combined TPE, brain TPE, and cancer TPE treatments (final average). Data shown as average of tested patients. The shaded box in the graph indicates the healthy range.

FIG. 21 shows a graph depicting changes in the level of the indicated volatile organic compound before (initial average) and 6 weeks after combined TPE, brain TPE, and cancer TPE treatments (final average). Data shown as average of tested patients. The shaded box in the graph indicates the healthy range.

FIG. 22 shows graphs depicting changes in the levels of the indicated myocotoxins before (initial average) and 6 weeks after combined TPE, brain TPE, and cancer TPE treatments (final average). Data shown as average of tested patients. The shaded box in each graph indicates the healthy range.

FIG. 23 shows graphs depicting changes in the levels of the indicated PFAS before (initial average) and 6 weeks after combined TPE, brain TPE, and cancer TPE treatments (final average). Data shown as average of tested patients. The shaded box in each graph indicates the healthy range.

FIG. 24 shows graphs depicting changes in the cognitive function test scores before (initial average) and 6 weeks after combined TPE, brain TPE, and cancer TPE treatments (final average). Data shown as average of tested patients. The shaded box in each graph indicates the healthy range.

FIG. 25 shows graphs depicting changes in the levels of the indicated cancer biomarkers before (initial average) and 6 weeks after combined TPE, brain TPE, and cancer TPE treatments (final average). PanCK=pancytokeratin. Data shown as average of tested patients.

### DETAILED DESCRIPTION

The disclosure relates to methods, compositions, and combinations for detoxification in a patient comprising performing therapeutic plasma exchange (TPE) on the patient. The disclosure also relates to methods, compositions, and combinations for removing toxins in a patient as a result of chronic exposure to the toxins, comprising performing one or more TPE sessions on the patient and administering an oral composition to the patient. The methods, compositions, and combinations of the disclosure may also be used to replenish biomarkers indicative of a healthy state of the body in order to improve immune system function or patient longevity, and/or reduce oxidative stress or inflammation. The methods, compositions, and combinations of the disclosure may also be used to reduce the level of atherosclerosis, improve cognitive function, prevent early cancer development, promote immune health, and/or treat health conditions or diseases such as autoimmune diseases, cancers, Alzheimer's disease, Parkinson's disease, chronic fatigue syndromes, or fibromyalgia.

In some embodiments of the methods, compositions, and combinations of the disclosure, plasma is removed from the patient at least once every 1 to 20 weeks after the last TPE session. In some embodiments of the methods, compositions, and combinations of the disclosure, plasma is removed from the patient at least once every 1 to 20 weeks after a TPE session is performed by donating plasma.

In some embodiments, the methods, compositions, and combinations of the disclosure comprise an additional intravenous supplementation, wherein each TPE session is followed by administration to the patient of an IV composition.

The disclosure also relates to oral compositions and IV compositions for use in the methods, compositions, and combinations of the disclosure. In some embodiments, the IV compositions of the disclosure comprise at least one mineral, at least one vitamin, and at least one amino acid. In some embodiments, the oral compositions of the disclosure comprise at least one vitamin, at least one mineral, at least one anti-inflammatory compound, at least one antioxidant, at least one amino acid, at least one saccharide, and at least one herb. In some methods, compositions, and combinations of the disclosure, the oral composition is administered to the patient daily after the first TPE session.

As utilized in accordance with the present disclosure, unless otherwise indicated or defined, all technical and scientific terms used herein shall be understood to have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Unless otherwise required by context, singular terms shall include pluralities and plural terms shall include the singular. For example, the terms “a,” “an,” and “the,” as used herein, are understood to be singular or plural unless the context clearly dictates otherwise. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives unless otherwise indicated. Thus, unless specifically stated or apparent from context, the term “or,” as used herein, is understood to be inclusive.

The terms “comprises” and “comprising,” as used herein, can have the meaning ascribed to them in U.S. patent law and can mean “includes,” “including,” “containing,” “having,” and the like. Thus, unless expressly specified otherwise, the terms “comprises” and “comprising,” as used herein, indicate that further components or members may optionally be present in addition to the components or members of the list introduced by “comprising.” The terms “consisting essentially of” or “consists essentially,” as used herein, likewise have the meaning ascribed in U.S. patent law, and allow for the presence of more than that which is recited so long as basic or novel characteristics of that which is recited are not changed by the presence of more than that which is recited.

Any of the methods, compositions, or combinations provided herein can be combined with one or more of any of the other methods, compositions, or combinations provided herein.

In the present disclosure, any concentration range, percentage range, ratio range, or integer range is to be understood to include the value of any integer within the recited range and, when appropriate, fractions thereof (such as one tenth and one hundredth of an integer), unless otherwise indicated. Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or apparent from context, the terms “about” and “approximately,” as used herein, are understood as meaning within a range of normal tolerance in the art, for example within 2 standard deviations of the mean, or mean within an acceptable error range for the particular value as determined by one of ordinary skill in the art, which will depend in part on how the value is measured or determined, i.e., the limitations of the measurement system. “About” can be understood as meaning within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

It is noted that terms like “generally,” “commonly,” “optionally,” and “typically” are not utilized herein to limit the scope of the claimed subject matter or to imply that certain features are critical, essential, or even important to the structure or function of the claimed subject matter. Rather, these terms are merely intended to highlight alternative or additional features that can or cannot be utilized in a particular embodiment of the present disclosure.

### 1. METHOD OF DETOXIFICATION WITH COMBINED THERAPEUTIC PLASMA EXCHANGE (TPE) TREATMENT

The disclosure provides methods, compositions, and combinations for detoxification in a patient, wherein the methods, compositions, and combinations can be used to remove toxins in the patient as a result of chronic exposure. The methods, compositions, and combinations of the disclosure can also be used to replenish biomarkers indicative of a healthy state of the body in order to improve immune system function or patient longevity, and/or reduce oxidative stress or inflammation. The methods, compositions, and combinations of the disclosure may also be used to reduce the level of atherosclerosis, improve cognitive function, prevent early cancer development, promote immune health, and/or treat health conditions or diseases such as autoimmune diseases, cancers, Alzheimer's disease, Parkinson's disease, chronic fatigue syndromes, or fibromyalgia.

In the methods, compositions, and combinations of the disclosure, one or more therapeutic plasma exchange (TPE) sessions is performed on the patient. In some embodiments, the methods, compositions, and combinations for detoxification described herein comprise: (a) performing one or more TPE sessions on the patient; and (b) administering an oral composition to the patient. In some embodiments, the oral composition is administered to the patient daily after the first TPE session. In some embodiments, plasma is removed from the patient at least once every 1 to 20 weeks after the last TPE session. The step of removing plasma from the patient can be performed by donating plasma.

As used herein, the term “therapeutic plasma exchange” or “TPE” refers to a procedure in which plasma is removed from the body and the plasma is replaced with a colloid solution. In TPE, the patient's blood is passed through an apheresis machine, where the filtered plasma is removed and replaced with reinfusion of red blood cells and colloid fluid containing plasma or albumin. TPE is typically used to eliminate plasma-bound proteins or antibodies that contribute to diseases. However, a series of TPE treatments has never been tested or described before for use in the removal of toxins from the body as a result of chronic exposure to the toxins. In some embodiments, between 2 and 8 TPE sessions are performed and each TPE session is performed between 1 and 6 weeks after the preceding TPE session.

The methods, compositions, and combinations for detoxification described herein involve patients donating plasma at a blood bank in which the plasma is filtered out from blood and removed from the body once every 1, 2, 3, 4, 5, or up to every 20 weeks but no more than once a week for no more than 3 weeks in a row, after completion of the TPE sessions. Removal of plasma is effective in maintaining the state of detoxification obtained from TPE sessions even after 6 months following completion of the TPE sessions (see Table 5).

In some embodiments of the methods, compositions, and combinations of the disclosure, IV or subcutaneous infusion of between 1 g to 20 g of immunoglobulin (or 10 mL to 200 mL of 10% immunoglobulin) may be administered to the patient after a TPE session is completed. In some embodiments, IV or subcutaneous infusion of 5 g of immunoglobulin is administered to the patient.

In some embodiments of the methods, compositions, and combinations for detoxification, a metal chelator medication may be taken orally by the patient following each TPE session. The metal chelator is to synergize with the TPE sessions to remove heavy metals in the body. In some embodiments, the metal chelator includes, but is not limited to, dimercaptosuccinic acid (DMSA), 2,3 dimercaptopropane sulphonate (DMPS), ethylenediaminetetraacetic acid (EDTA), sodium calcium edetate, D-penicillamine, or N-acetyl-DL-penicillamine. The metal chelator may be administered by IV or orally. In some embodiments, the metal chelator is DMSA. In some embodiments, 50 mg to 2000 mg DMSA is administered orally to the patient for 1, 2, 3, or 4 days a week. In some embodiments, 250 mg DMSA is administered to the patient for 3 consecutive days and stopped for the next 4 days, every week after the first TPE is completed. In some embodiments, 50 mg to 500 mg DMSA is administered intravenously to the patient following a TPE procedure and no more than once per week. In some embodiments, the metal chelator is EDTA, wherein the EDTA may be administered by IV at a dose of between 500 and 5000 mg, or administered orally at a dose of between 500 and 3000 mg. In some embodiments, the metal chelator is DMPS, wherein the DMPS may be administered by IV at a dose of between 500 and 250 mg, or administered orally at a dose of between 100 and 500 mg. In some embodiments, DMSA is administered orally to the patient at 250 mg for the first 3 consecutive days in a week (Monday through Wednesday), every week for 1 to 52 weeks.

In some embodiments, patients are advised to make lifestyle changes to avoid exposure to toxins such as air filtration, water filtration, food selection and preparation, and elimination of household chemical toxins.

In some embodiments, the methods, compositions, and combinations of the disclosure further comprise administration of an IV composition. In some embodiments, the methods, compositions, and combinations of the disclosure further comprise administration of an IV composition and glutathione solution. The IV composition is administered to the patient immediately after the first TPE session is completed. In some embodiments, the administration of an oral composition is initiated after the first TPE session with IV composition and glutathione solution administration is completed and maintained at least until all TPE sessions are completed. The administration of an oral composition may continue after all TPE sessions are completed. The number of TPE sessions and the time intervals between the TPE sessions can be adjusted depending on the level of toxins in a patient before the treatment and the general health of the patient.

In some embodiments, patients are optionally subjected to one or a series of laboratory tests to determine the pre-treatment biomarker and/or toxin levels, and optionally one or more laboratory tests after each cycle of the methods disclosed herein has been completed to determine the effectiveness of the treatment. Such tests can be scheduled one or two weeks before the first TPE and after the last TPE has been completed. Samples collected from patients for testing can include urine and/or blood. Patients can be given a metal chelator medication before each laboratory test. Metal chelators are metal-binding ligands specific for each metal forming a ring-structure called a chelate. In some embodiments, the metal chelator includes, but is not limited to, dimercaptosuccinic acid (DMSA), 2,3 dimercaptopropane sulphonate (DMPS), ethylenediaminetetraacetic acid (EDTA), sodium calcium edetate, D-penicillamine, or N-acetyl-DL-penicillamine. In some embodiments, the metal chelator is DMSA and the DMSA is given at a dose of between 50 mg and 2000 mg. In certain embodiments, the DMSA is given at a dose of 1500 mg.

As used herein, the terms “combined TPE” or “combined TPE treatment” refer to the methods, compositions, and combinations for detoxification described herein with oral supplementation. Specifically, the combined TPE treatment comprises: (a) performing one or more TPE sessions on the patient; and (b) administering an oral composition to the patient. In some embodiments, the combined TPE treatment further comprises administering an intravenous (IV) composition to the patient and administering a glutathione solution to the patient after each TPE session has been performed on the patient. In some embodiments, plasma is removed from the patient at least once every 1 to 20 weeks after the last TPE session.

As used herein, the term “combined TPE” is intended to distinguish the methods, compositions, and combinations for detoxification with additional IV and/or oral supplementation disclosed herein from control TPE treatment (referred to herein as “TPE only” or “TPE only treatment”) comprising the methods, compositions, and combinations for detoxification without additional IV and oral supplementation.

As described herein, in combined TPE treatment, the oral composition is administered to the patient daily after the first TPE session. After all TPE sessions have been completed, administration of the oral composition to the patient continues daily for 2, 4, 6, 8, 10, 12 months or longer in order to maintain the state of detoxification achieved by the combined TPE.

It has been shown that chronic exposure to toxins (especially heavy metals) even at low levels, can result in severe health consequences, including various types of cancers, kidney damage, neuropsychiatric disorders, neurodegenerative diseases, and cardiovascular disease (Briffa et al., Heliyon 6(9): e04691 (2020); Brylinski et al., Int. J. Mol. Sci. 24(8): 7228 (2023)). The combined TPE treatment described herein is not only capable of reducing the toxin levels but is also effective at halting the progression of atherosclerosis, improving cognitive function, reducing circulating tumor cells or preventing early development of cancer, and/or improving immune health.

In some embodiments, combined TPE treatment is used to halt the progression of atherosclerosis. Atherosclerosis is a common condition that develops when plaque builds up inside the artery. In some embodiments, the combined TPE treatment is used to halt the progression of atherosclerosis in a patient by lowering the oxidative stress and/or inflammation. These patients would otherwise require bypass surgery because they have been maximized on medications, meaning that the medications have exerted their maximal effects, but the plaque is still developing, or the patients or physicians do not wish to continue or increase the medications due to side effects or for other reasons. In other words, there are no other viable medication options remaining for these patients.

In some embodiments, the methods, compositions, and combinations of the disclosure are used to reduce the level of atherosclerosis. Such methods, compositions, and combinations, referred herein as “heart TPE,” comprise combined TPE plus administration of at least one cholesterol-reducing agent after the first TPE session and administration of regenerative materials. As used herein, the term “cholesterol-reducing agent” refers to a compound that lowers the cholesterol level in the body. The at least one cholesterol-reducing agent is a drug that has not been administered to the patient prior to the patient receiving heart TPE treatment as described herein. In some embodiments, the cholesterol-reducing agent is beta-cyclodextrin. In some embodiments, beta-cyclodextrin can be administered to the patient as an IV or as an enema daily for 4 to 12 months after the first TPE session. In some embodiments, beta-cyclodextrin is administered at a dose of between 8 g and 16 g, as frequently as once a day, or as infrequently as twice a week, for 6 months after the first TPE session. In some embodiments, beta-cyclodextrin is administered at a dose of 8 g once a day for 6 months after the first TPE session.

In some embodiments, the methods, compositions, and combinations of the disclosure are used to improve cognitive function in a patient. Such methods, compositions, and combinations, referred herein as “brain TPE,” comprise combined TPE plus administration of neuropeptides and regenerative materials. Brain TPE may be used to improve cognitive function in patients with cognitive decline.

Neuropeptides can regulate gene expression and secretion of growth factors that stimulate neuronal survival and growth (Burger et al., J. Biol. Chem. 291(31): 16293-306 (2016); Kaplan et al., Neurosci. Res. Commun. 19(2): 115-23 (1996)). Neuropeptides are administered to the patient before at least one TPE session but no more than 2 consecutive TPE sessions or before the first TPE session and are maintained throughout all the rest of the TPE sessions. Examples of neuropeptides include, but are not limited to, an adrenocorticotropin such as SEMAX; a tetrapeptide having the amino acid sequence AEDG (SEQ ID NO: 1) such as epitalon; a heptapeptide having the amino acid sequence TKPRPGP (SEQ ID NO: 2) such as selank; a tripeptide having the amino acid sequence EDR such as pinealon; a tripeptide having the amino acid sequence KED such as vesugen; a fibroblast growth loop peptide (FGL), CEREBROLYSIN which includes a mixture of brain-derived neurotrophic factor (BDNF), glial cell line-derived neurotrophic factor (GDNF), nerve growth factor (NGF), and ciliary neurotrophic factor (CNTF); hypocretin-1; low molecular peptides with a molecular weight up to 5000 Da isolated from the brain tissues of calves, CERLUTEN, or low molecular peptides with molecular weight up to 10000 Da isolated from the vascular (aorta) tissue of calves, VENTFORT. In some embodiments, the neuropeptides are adrenocorticotropin (4-10) SEMAX and/or synthetic tetrapeptide epitalon having the amino acid sequence of SEQ ID NO: 1. In some embodiments, adrenocorticotropin (4-10) SEMAX is administered via intranasal spray at a dose of between 1 mcg to 2000 mcg. In some embodiments, adrenocorticotropin (4-10) SEMAX is administered via intranasal spray at a dose of 750 mcg. In some embodiments, administration of adrenocorticotropin (4-10) SEMAX is initiated 5 to 7 days before the first TPE session and is maintained until the last TPE session is completed. In some embodiments, synthetic tetrapeptide epitalon is administered via subcutaneous injection or oral spray at a dose of between 0.1 and 50 mg. In some embodiments, synthetic tetrapeptide epitalon is administered via subcutaneous injection at a dose of 5 mg. In some embodiments, administration of synthetic tetrapeptide epitalon is initiated 5 to 7 days before the first TPE session and before every other TPE session thereafter (e.g., 3rd TPE, 5th TPE, and 7th TPE) and maintained daily for 5 to 10 consecutive days.

As used herein, the term “regenerative materials” refers to biological materials such as molecules, tissue or cell-derived components, and cells that have regenerative properties. Examples of regenerative materials include, but are not limited to, stem cells, cytokines, growth factors, exosomes, nucleic acids, or natural nanoparticles or proteins that have regenerative properties. In some embodiments, the regenerative materials are mesenchymal stem cells and exosomes. In some embodiments, umbilical cord product which is known to contain stem cells and other regenerative materials can also be used. In some embodiments, the regenerative materials are administered to the patient after at least one TPE session but no more than 2 consecutive TPE sessions. In some embodiments, a Wharton's Jelly allograft contained in Vitti Labs WJ-PURE PLUS 1 is administered via intranasal spray at a dose of between 0.1 mL and 10 mL. WJ-PURE PLUS 1 is a minimally manipulated, bio-ethical human umbilical cord harvested product from donated human tissue manufactured under GTP, cGMP, AATB, and FDA HCT/P 361 guidelines for homologous use in clinical settings. WJ-PURE PLUS 1 is DMSO free, and the harvesting process is absent of harmful enzymes and chemicals to preserve integrity. In some embodiments, a Wharton's Jelly allograft contained in Vitti Labs WJ-PURE PLUS 1 is administered via intranasal spray at a dose of 1 mL. Wharton's Jelly allograft comprises stem cells, hyaluronan, elastin, collagen fibers, cytokines, growth factors, natural nanoparticles or proteins with regenerative properties. In some embodiments, the Wharton's Jelly allograft contained in Vitti Labs WJ-PURE PLUS 1 is administered immediately after the first TPE session and every other TPE session thereafter (e.g., 3rd TPE, 5th TPE, and 7th TPE). In some embodiments, exosomes, growth factors, cytokines, and nucleic acids contained in Vitti Pure EV PURE PLUS 1 is administered via intranasal spray and/or intravenous injection at a dose of between 0.1 mL and 10 mL. Vitti Pure EV PURE PLUS 1 is a minimally manipulated, bio-ethical human umbilical cord harvested product from donated human tissue manufactured under GTP, cGMP, AATB, and FDA HCT/P 361 guidelines. Vitti Pure EV PURE PLUS 1 is DMSO free, and the harvesting process is absent of harmful enzymes and chemicals to preserve integrity. In some embodiments, the exosomes, growth factors, cytokines, and nucleic acids contained in Vitti Pure EV PURE PLUS 1 are administered via intranasal spray at a dose of 1 mL and/or intravenous injection at a dose of between 2 to 4.5 mL. In some embodiments, the exosomes, growth factors, cytokines, and nucleic acids contained in Vitti Pure EV PURE PLUS 1 is administered via intranasal spray at a dose of 1 mL and intravenous injection at a dose of 3.5 mL. In some embodiments, the exosomes, growth factors, cytokines, and nucleic acids contained in Vitti Pure EV PURE PLUS 1 is administered immediately after the first TPE session and every other TPE session thereafter (e.g., 3rd TPE, 5th TPE, and 7th TPE).

In some embodiments, the methods, compositions, and combinations of the disclosure are used to reduce circulating tumor cells or prevent early development of cancer. Such methods, compositions, and combinations, referred herein as “cancer TPE,” comprise combined TPE plus administration of at least one anti-inflammatory compound, one anti-fungal compound, or a combination thereof. It has been hypothesized that cancer is caused and propagated by inflammation. Anti-inflammatory medications can slow or reverse the development of cancer; while anti-fungal medications can reduce inflammation caused by fungus (Weng et al., J. Adv. Res. 48: 259-73 (2023); Thun et al., J. Natl. Cancer Inst. 94(4): 252-66 (2002)). Cancer TPE may be used to reduce circulating tumor cells in the peripheral blood and reverse expression of biological markers in peripheral mononuclear cells (PBMC), which are indicative of early cancer development.

In some embodiments, after completion of the first TPE, patients are administered thymosin al, met enkephalin, methylene blue, nystatin, diflucan, colloidal silver, and mebendazole. In some embodiments, thymosin al is injected subcutaneously to the patient at a dose of between 0.5 mg and 3 mg once daily for 1 to 52 weeks. In some embodiments, thymosin al is injected subcutaneously to the patient at a dose of 1.6 mg once daily for 1 to 52 weeks. In some embodiments, met enkephalin is injected subcutaneously to the patient at a dose of between 0.5 mg and 3 mg once daily for 1 to 52 weeks. In some embodiments, met enkephalin is injected subcutaneously to the patient at a dose of 1.5 mg once daily for 1 to 52 weeks. In some embodiments, methylene blue is taken orally by the patient at a dose of between 1 mg and 100 mg once daily for 1 to 52 weeks. In some embodiments, methylene blue is taken orally by the patient at a dose of 50 mg once daily for 1 to 52 weeks. In some embodiments, nystatin liquid is taken orally by the patient at a dose of between 10,000 units and 4,000,000 units four times a day for 1 to 52 weeks. In some embodiments, nystatin liquid is taken orally by the patient at a dose of 500,000 units four times a day for 1 to 52 weeks. In some embodiments, Diflucan is taken orally by the patient at a dose of between 50 mg and 200 mg once a day for 1 to 52 weeks. In some embodiments, Diflucan is taken orally by the patient at a dose of 100 mg once daily for 1 to 52 weeks. In some embodiments, colloidal silver 2000 ppm is taken orally by the patient at a dose of between 1 mL and 100 mL four times a day for 1 to 52 weeks. In some embodiments, colloidal silver 2000 ppm is taken orally by the patient at a dose of 10 mL four times a day for 1 to 52 weeks. In some embodiments, mebendazole is taken orally by the patient at a dose of between 50 mg and 200 mg once daily for 1 to 52 weeks. In some embodiments, mebendazole is taken orally by the patient at a dose of 200 mg once daily for 1 to 52 weeks.

In some embodiments, the methods, compositions, and combinations of the disclosure are used to improve immune health. Such methods, compositions, and combinations, referred herein as “immunity TPE,” comprise combined TPE plus administration of regenerative materials. The administration of the regenerative materials is similar to that described herein for brain TPE and heart TPE.

In some embodiments, brain TPE, cancer TPE, or immunity TPE comprises administration of low dose of naltrexone (LDN). Naltrexone is an opioid antagonist that can act as an anti-inflammatory agent in low dose (Younger et al., Clin. Rheumatol. 33(4): 451-59 (2014)). In some embodiments, naltrexone is taken orally by the patient after the first TPE is completed at a dose of between 1 mg and 20 mg once daily for 1 to 52 weeks. In some embodiments, naltrexone is taken orally by the patient after the first TPE is completed at a dose of 1.5 mg once daily for 2 weeks, and then the dose is increased to 3 mg once daily for the next 2 weeks, and the dose subsequently increased to 4.5 mg once daily for the next 11 to 19 weeks.

As used herein, the term “detoxification” refers to the removal of toxic substances from a living organism. As used herein the term “medical procedure” refers to a series of actions performed on a patient to achieve an intended result in the delivery of healthcare. The medical procedure performed herein can include TPE followed by intravenous infusion of a composition comprising supplements and nutrients such as minerals, vitamins, and amino acids, which is followed by an infusion of a strong antioxidant such as a glutathione solution. The aim of the medical procedure described herein is to remove toxins from the patient and supply the patient with substances through IV infusion that are beneficial for the health of the patient.

As used herein, the term “chronic exposure” refers to prolonged or repeated contact with a substance over a long period of time, such as months or years. The amount of the contacted substance may be sub-lethal or minimal after each exposure but can build up in the body over time. The built-up substance might not show immediately, and in some cases, symptoms of the toxic exposure may gradually contribute to physiological disorders and diseases. Furthermore, as the substance is present in the body for a long period of time, it might be incorporated into blood and tissues that make access to and removal of the substance difficult. This is in contrast with “acute exposure” which involves contact with an excessive or toxic dose of substance over a short period to time, such as seconds, hours, or days. An acute exposure of a toxic substance can lead to poisoning symptoms that require immediate treatment in order to remove the substance.

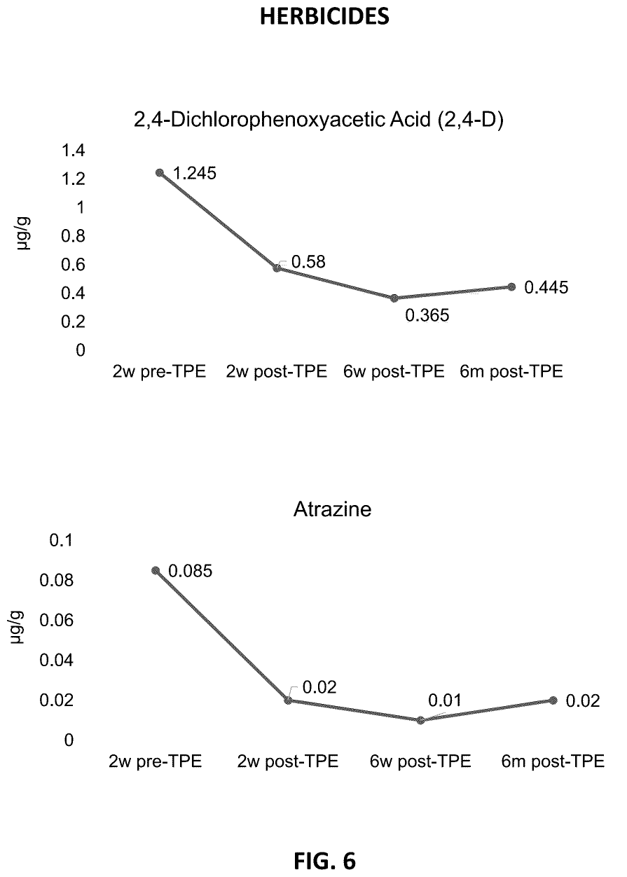

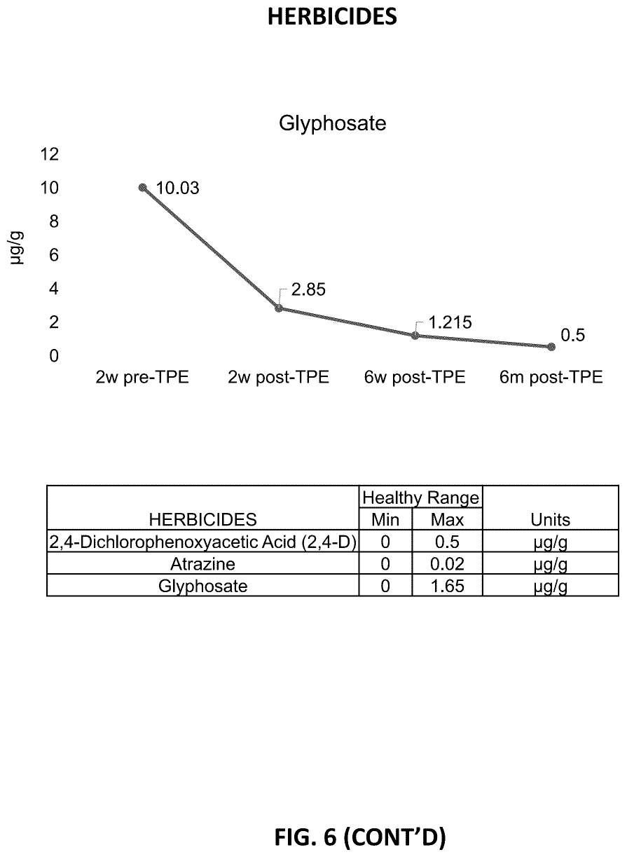

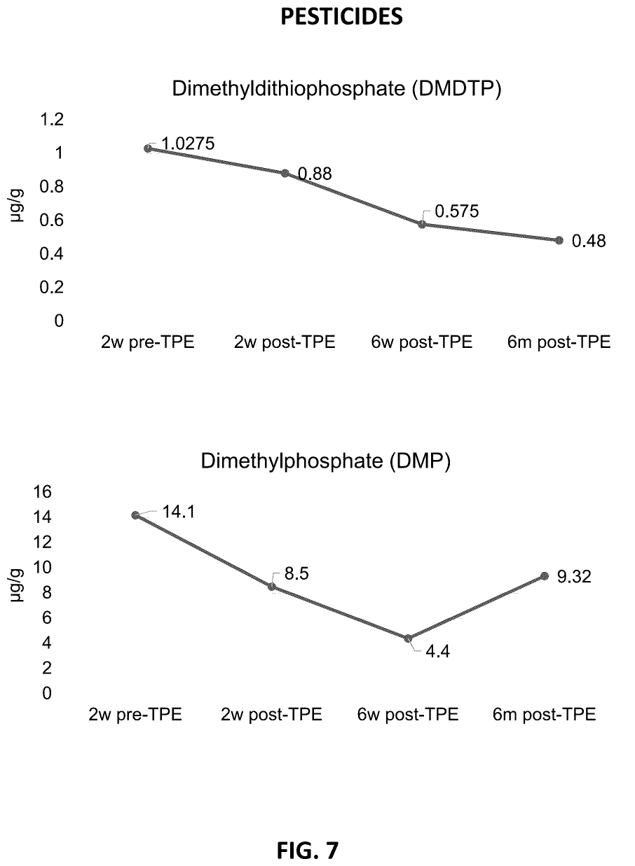

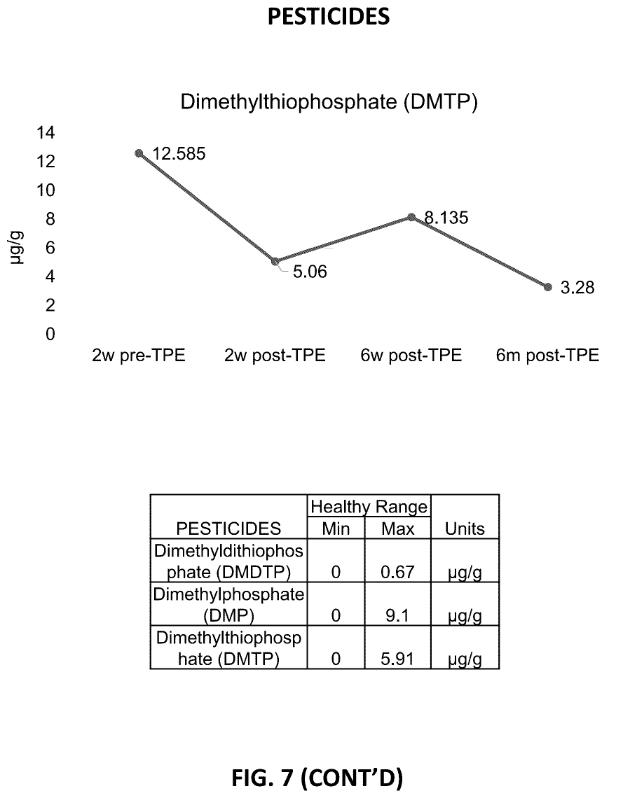

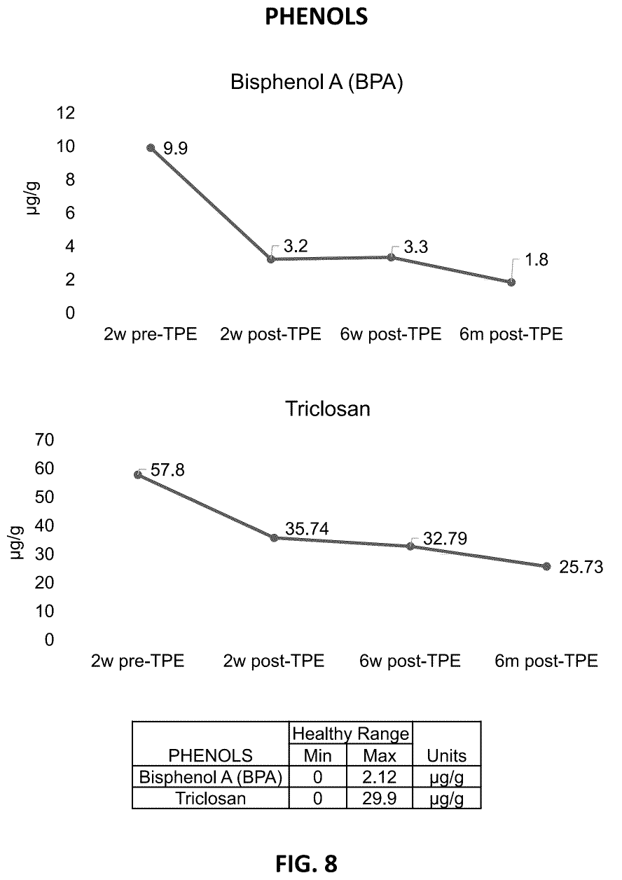

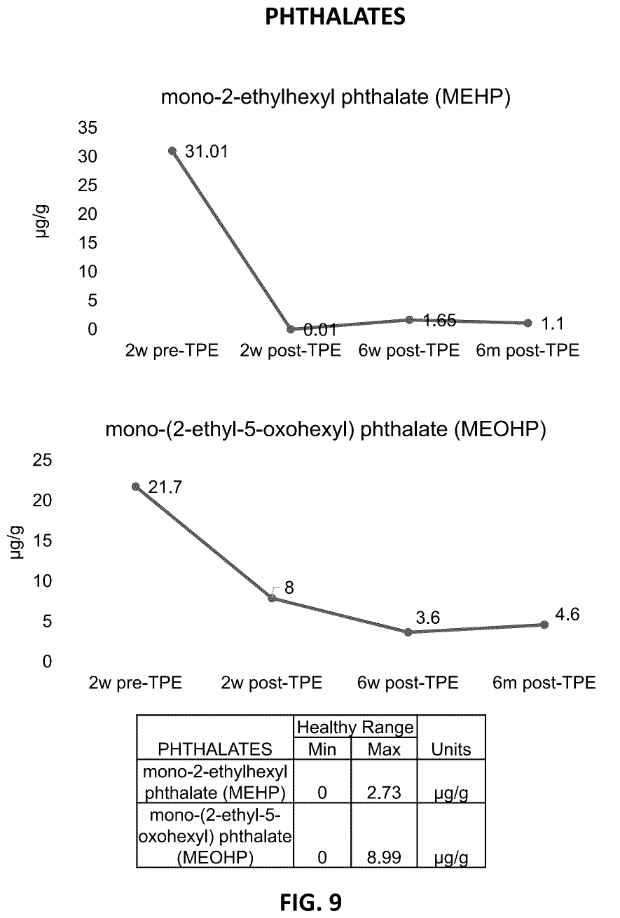

As described in Example 4 and as shown in FIGS. 6-13 and Tables 5 and 6, combined TPE treatment and TPE only treatment reduce many tested toxins from a high or abnormal level before treatment to a normal level after treatment. The toxic substances, or “toxins,” include substances that are naturally occurring or that are produced by or as a by-product of non-natural (synthetic) activities. Such toxins can be introduced into the patient from the environment via diet, inhalation, ingestion, skin contact, or by other routes. Toxins can also be produced within the patient. Toxins can include, but are not limited to, heavy metals such as nickel, aluminum, arsenic, palladium, cadmium, antimony, tin, tellurium, cesium, barium, gadolinium, tungsten, platinum, mercury, lead, thallium, bismuth, thorium, uranium; and chemical toxins such as herbicides (e.g., atrazine, glyphosate); pesticides (e.g., 2,2-bis(4-chlorophenyl) acetic acid (DDA), diethyldithiophosphate (DEDTP), dimethylphosphate (DMP)); phenols (e.g., triclosan, bisphenol A (BPA)); phthalates (e.g., mono-(2-ethyl-5-hydroxyhexyl) phthalate (MEHHP), mono-(2-ethyl-5-oxohexyl) phthalate (MEOHP)); volatile organic compounds (N-acetyl-S-(2-carbamoylethyl)-cysteine (NAE), 2-hydroxyisobutyric Acid (2HIB), phenylglyoxylic Acid (PGO)); and mycotoxins (e.g., aflatoxin B2, ochratoxin, zearalenone). Heavy metals have been shown to disrupt cellular events including growth, proliferation, differentiation, damage-repairing processes, and apoptosis. Their mechanisms of action can include generation of reactive oxidative species (ROS) and oxidative stress, selective binding to macromolecules, and genomic instability, which contribute to carcinogenicity (Balali-Mood et al., Front. Pharmacol. 12: 643972 (2021); Jaishankar et al., Interdiscip. Toxicol. 7(2): 60-72 (2014); Witkowska et al., Molecules 26(19): 6060 (2021)). Meanwhile, evidence of causal links between chemical toxins and diseases have been strongest in obesity and diabetes, reproduction, hormone-sensitive cancers in females, prostate, thyroid, neurodevelopment, neuroendocrine systems (Gore et al., Endocr. Rev. 36(6): E1-E150 (2015)), and immune dysregulation (Masri et al., Environ. Sci. Eur. 33: 65 (2021)).

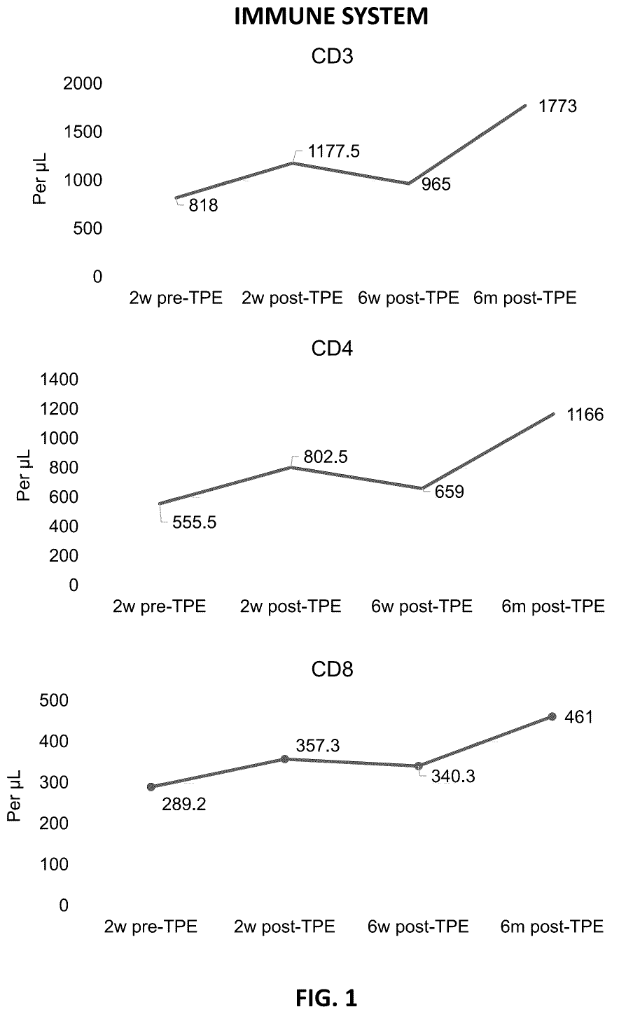

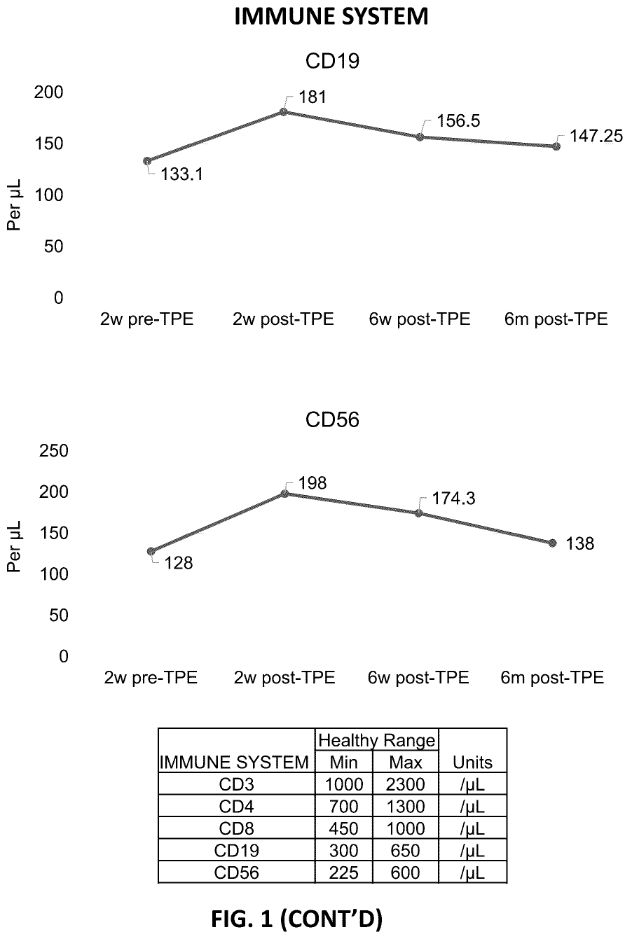

As described in Examples 2 and 3 and as shown in FIGS. 1-4 and Table 5, combined TPE treatment and TPE only treatment increase many biomarkers that are indicative of an improvement in immune system and longevity. Examples of the former include, but are not limited to, CD3, CD4, CD8, CD19, and CD56 (Hoi et al., Exp. Gerontol. 1: 176: 112163 (2023); Hashimoto et al., Proc. Natl. Acad. Sci. U.S.A. 116(48): 24242-51 (2019)). As used herein, the term “biomarkers of longevity” or “biomarkers of aging” are biomarkers that could predict functional capacity at some later age better than chronological age (Baker et al., Exp. Gerontol. 23(4-5): 223-39 (1988)). In other words, biomarkers of aging would give the true “biological age,” which may be different from the chronological age. Examples of biomarkers of longevity include, but are not limited to, Klotho (Cheikhi et al., J. Gerontol. A Biol. Sci. Med. Sci. 74(7): 1031-42 (2019); Correa et al., Sci. Rep. 12(1): 17587 (2022)), nicotinamide adenine dinucleotide intracellular (NAD intracellular) (Kincaid et al., Exp. Biol. Med. (Maywood) 245(17): 1594-614 (2020)), reactive oxygen metabolite-derived compound (ROM) (Ristow et al., Exp. Gerontol. 45(6): 410-18 (2010)), total antioxidant capacity (TAC) (Wojcik et al., Curr. Med. Chem. 17(28): 3262-88 (2010)), albumin (Soeters et al., JPEN J. Parenter. Enteral. Nutr. 43(2): 181-93 (2019)), alkaline phosphatase (ALP) (Kunutsor et al., Int. J. Epidemiol. 43(1): 187-201 (2014)), alanine transaminase (ALT) (Couteur et al., J. Gerontol. A Biol. Sci. Med. Sci. 65(7): 712-17 (2010)), creatine kinase (Smith et al., F1000Res. 3: 222 (2014)), estimated glomerular filtration rate (eGFR) (Ortiz et al., Nephrol. Dial. Transplant. 38(3): 523-26 (2022)), glycated serum protein (GSP) (Dall'Olio et al., Ageing Res. Rev. 12(2): 685-98 (2013)), high density lipoprotein (HDL-C) (Hong et al., Int. J. Mol. Sci. 24(20): 15305 (2023)), low density lipoprotein (LDL-C) (Pietri et al., J. Am. Coll. Cardiol. 77(2): 188-204 (2021)), triglycerides (Johnson et al., Aging Cell. 18(6): e13048 (2019)), uric acid (Li et al., BMJ357: j2376 (2017)), vitamin D3 (Berridge, J. Physiol. 595(22): 6825-36 (2017); Schottker et al., J. Gerontol. A Biol. Sci. Med. Sci. 74(1): 121-28 (2019)), high sensitivity C-reactive protein (hs-CRP) (Tang et al., Clin. Exp. Pharmacol. Physiol. 44(Suppl 1): 9-14 (2017); Li et al., Atherosclerosis 259: 75-82 (2017)), senescence-associated beta galactosidase (SA-j-gal) (Sikora et al., Curr. Vasc. Pharmacol. 12(5): 698-706 (2014)); Kamal et al., Eur. J. Cell. Biol. 99(6): 151108 (2020)), interleukin-10 (IL-1) (Franceschi et al., Mech. Ageing Dev. 128(1): 92-105 (2007); Frangogiannis, Discoveries (Craiova) 3(1): e41 (2015)), interleukin 6 (IL-6) (Maggio et al., J. Gerontol. A Biol. Sci. Med. Sci. 61(6): 575-84 (2006)), interleukin 8 (IL-8) (Apostolakis et al., Cardiovascular Res. 84(3): 353-60 (2009)), tumor necrosis factor α (TNFα) (Minciullo et al., Arch. Immun. Thera. Exp. (Warsz.) 64(2): 111-26 (2016); Bruunsgaard et al., Am. J Med. 115(4): 278-83 (2003)), 8-Oxoguanine or 8-Oxo-2′-deoxyguanosine (8-OHdG or 8-oxo-dG) (Marasco et al., Mech. Ageing Dev. 164: 100-07 (2017); Di Minno et al., Antioxid. Redox Signal. 24(10): 548-55 (2016)), and myeloperoxidase (MPO) (Siraki, Redox Biol. 46: 102109 (2021)).

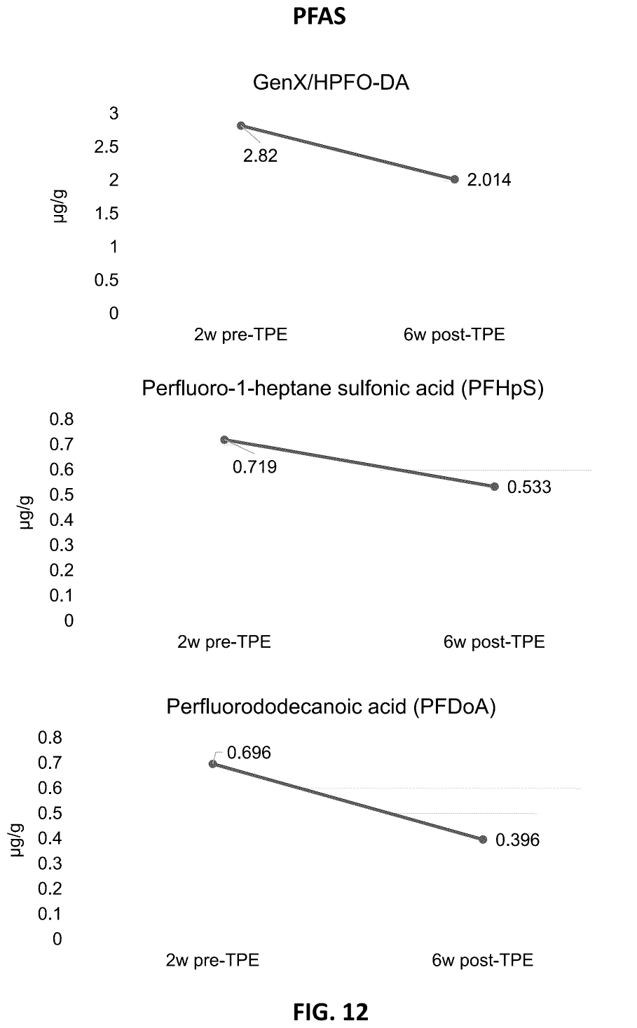

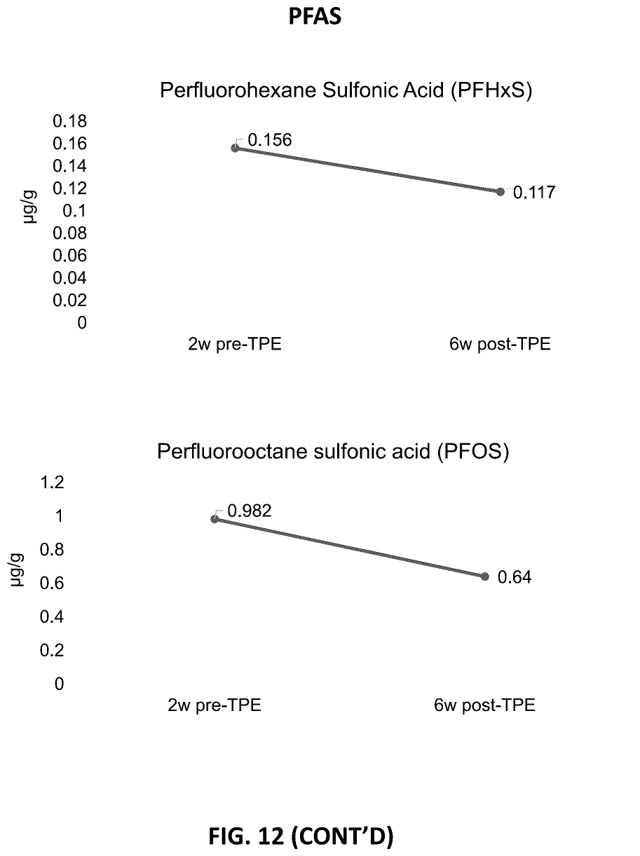

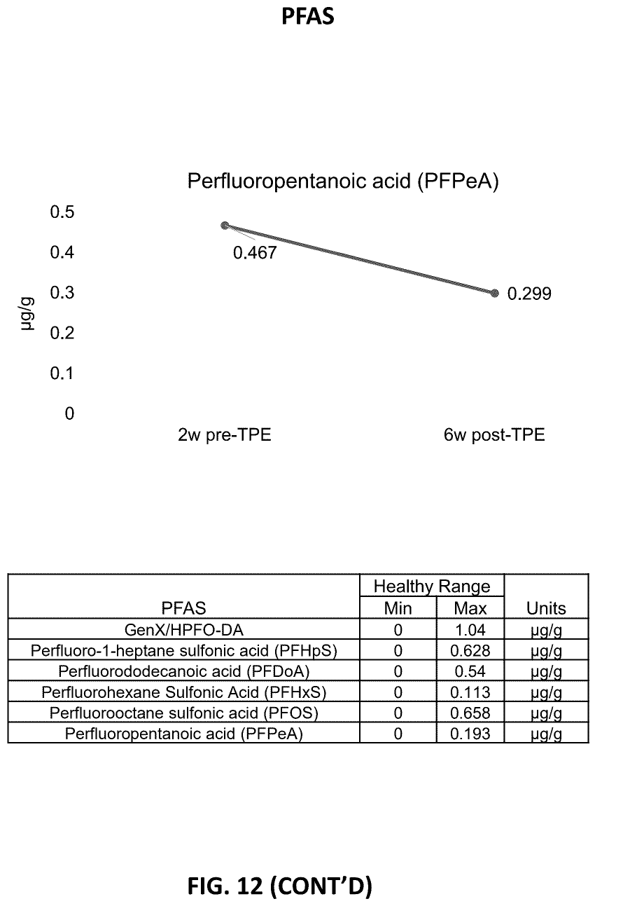

As described herein, the removal of toxins and increase in biomarkers of immune system and longevity following combined TPE treatment and TPE only treatment are sustained for 2 weeks, for 6 weeks, or for 6 months after treatment. This demonstrates that the results are not acute and that the methods, compositions, and combinations disclosed herein can offer prolonged benefits to the well-being of a patient. Furthermore, combined TPE treatment and TPE treatment were surprisingly effective in removing a wide variety of toxins, which is not observed or reported for any currently available detoxification methods. Based on the results described herein, the methods, compositions, and combinations described herein utilizing combined TPE and TPE only can be used to remove any type of environmental or man-made toxin. As shown in Example 5, FIG. 12, and Table 7, combined TPE can remove per- and polyfluorinated substances (PFAS) for which no approved detoxification method is available.

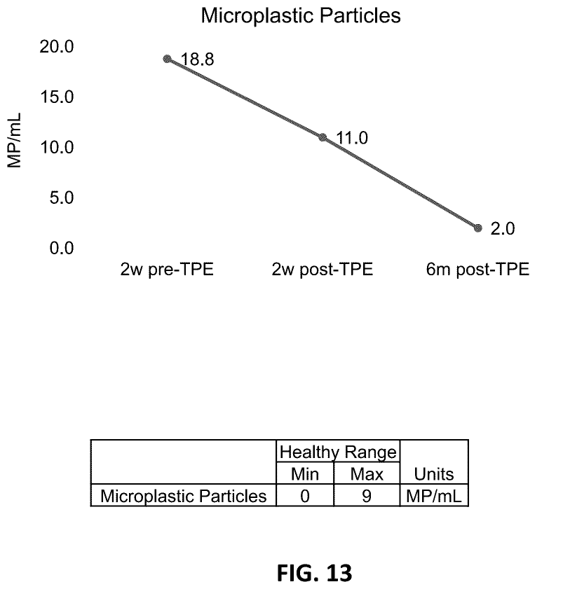

The term “microplastics” is used to describe fragments of any type of plastic less than 5 mm or 0.2 inches in length. As shown in Example 6, FIG. 13, and Table 8, combined TPE treatment is highly effective in removing microplastics such as polyamide, polyethylene (PE), polypropylene, polyethylene terephthalate, polytetrafluoroethylene, polymethyl methacrylate (PMMA), peroxyacyl nitrate (PAN), polyether ether ketone (PEEK), polylactic acid (PLA), polyoxymethylene (POM), poly(p-oxybenzoyl) (POB), polyisoprene, polyurethane (PU), polycarbonate (PC), polyether block amide (PEBA), silicone/polysiloxanes, poly(p-hydroxybutyrate, and other bio-derived and biodegradable plastics.

It is reported that chronic exposure to low levels of heavy metals increases the risk of cardiovascular diseases (American Heart Association News, “Higher cardiovascular risk linked to toxic metals found in everyday life,” https://www.heart.org/en/news/2023/06/12/higher-cardiovascular-risk-linked-to-toxic-metals-found-in-everyday-life, published Jun. 12, 2023, last accessed Sep. 18, 2024). Excessive oxidative stress (Cioffi et al., J. Alzheimers Dis. 72(4): 981-1017 (2019)), neuroinflammation (Rajesh et al., Cells 11(12): 1885 (2022)), and environmental toxins (Pamphlett et al., J. Alzheimers Dis. 45(2): 437-47 (2015); Mazzatenta et al., Respir. Physiol. Neurobiol. 209: 81 (2015)) also cause cognitive decline. Example 7 and Example 8 demonstrate that the methods, compositions, and combinations for detoxification described herein are effective in improving cognitive function and halting the progress of atherosclerosis in coronary arteries in patients who are already maximized on medications.

Patients who are suitable candidates for combined TPE treatment or TPE only treatment may or may not have one or more health conditions or diseases. For healthy patients, combined TPE treatment and TPE only treatment can lower toxins, boost immune function, and prevent disease development, thereby promoting patient well-being and longevity. Because of aging-related immune changes and accumulation of toxins over time, it is contemplated that healthy patients over 50 years old would benefit greatly from the methods, compositions, and combinations disclosed herein. However, the methods, compositions, and combinations of the disclosure can be used with any patient over the age of 18 years. For patients with clinical symptoms or diseases, the methods, compositions, and combinations disclosed herein utilizing combined TPE or TPE only detoxification can be used together with other procedures to remove pathogenic proteins and assist with the recovery and healing process. The methods, compositions, and combinations of the disclosure may also be used for the treatment of diseases including, but are not limited to, autoimmune diseases, cancers, Alzheimer's disease, Parkinson's disease, chronic fatigue syndromes, and fibromyalgia.

### 2. THERAPEUTIC PLASMA EXCHANGE (TPE)

The methods, compositions, and combinations disclosed herein utilize combined TPE treatment or TPE only treatment, which comprises therapeutic plasma exchange (TPE).

TPE has been used to treat a variety of autoimmune, hematological/oncological, metabolic, neurological, and renal disorders (Connelly-Smith et al., J. Clin. Apher. 38(2): 77-278 (2023)). In these instances, the goal of TPE is two-fold: to collect nearly cell-free plasma from the patient and replace the removed plasma with a physician-specified replacement fluid. Removing the patient's plasma removes all components that are carried by the plasma, which include but are not limited to, water, salts, sugars, fats, enzymes, nutrients, hormones, proteins (both vital and pathogenic), antibodies, clotting factors, foreign bodies, toxins (both endogenous and exogenous), and waste products. Removing the patient's plasma may modify/reduce the patient's symptoms caused by the pathogenic and toxic substances. Meanwhile, the replacement fluid is used to maintain the appropriate intravascular volume, the appropriate plasma protein concentration and replace a missing component in the plasma. Recently, there have been reports that have focused on the use of TPE to reverse biomarkers of aging (Kim et al., GeroScience 44(6): 2701-20 (2022)) and its association with aging and disease (Malchesk, Ther. Apher. Dial. 22(4): 312-16 (2018)). While TPE has also been used for detoxification, TPE is known to be effective with only certain toxins and in acute intoxication (Nenov et al., Nephrol. Dial. Transplant 18(Suppl5): v56-v58 (2003); Yilmaz et al., Am. J. Emerg. Med. 31(6): 953-57 (2013)).

Despite the above reports, however, there have been no studies assessing the effectiveness of TPE in removing toxins following long-term and/or slow exposure to such toxins. Some toxins, such as heavy metals, are known to be notoriously difficult to eliminate from the body (Rajkumar et al., “Heavy Metal Toxicity,” in StatPearls [Internet](Treasure Island (FL), StatPearls Publishing, 2024); Soloway, “Chelation: Therapy or ‘Therapy’?” <www.poison.org/articles/chelation-therapy> accessed Mar. 5, 2024). Thus, there remains a need for effective and safe methods, compositions, and combinations of detoxification for long-term and slow exposure to a wide variety of toxins. Long-term exposure to toxins is typically non-deliberate via numerous routes that include, but are not limited to, skin contact, inhalation, or digestion of toxins contaminated in daily food, air, or other consumer products. These types of exposure occur slowly and involve low doses of toxins that do not result in immediate mortality, but which accumulate an abnormal level of toxins over a longer period of time as compared to high and acute exposure. Nevertheless, accumulated toxins in the body (at sub-lethal but abnormal levels) have been known to be associated with accelerated aging (van der Laan et al., Environ. Int. 158: 106871 (2022); Wise et al., Front. Aging 3: 1014675 (2022)) and underlie many diseases such as cancer (National Cancer Institute, “Cancer-Causing Substances in the Environment,”<www.cancer.gov/about-cancer/causes-prevention/risk/substances> last accessed Mar. 8, 2024), Alzheimer's disease (Vasefi et al., Neurochem. Int. 141: 104852 (2020)), and heart disease (American Heart Association, “Higher cardiovascular risk linked to toxic metals found in everyday life,”<www.heart.org/en/news/2023/06/12/higher-cardiovascular-risk-linked-to-toxic-metals-found-in-everyday-life> last accessed Mar. 8, 2024). Furthermore, unlike acute exposure and emergency intoxication, where the toxins are limited to one or a few types and are clearly identified in most cases, long-term and slow exposure is non-deliberate in nature and is associated with many potential toxins. Thus, the latter exposure requires methods, compositions, and combinations of detoxification that are safe and effective in removing a wide range of toxins.

The methods, compositions, and combinations of detoxification disclosed herein utilize one or more TPE treatments alone (TPE only treatment) or TPE treatment in combination with IV infusion and/or oral administration of supplements (combined TPE treatment). Without being bound by any mechanism of action, the methods, compositions, and combinations of detoxification disclosed herein utilize several sessions of TPE with resting time intervals between those sessions, which allows for the equilibration and removal of all substances, including toxins, residing in intravascular (plasma) and extravascular (outside plasma) space. Thus, by employing multiple sessions of treatment, toxins can be widely eliminated from a patient. As described in Examples 2 and 3 and shown in FIGS. 1-5 and Table 5, combined TPE treatment and TPE only treatment increase many biomarkers that are indicative of an improvement in immune system and longevity. However, TPE non-selectively removes pathogenic proteins as well as other beneficial nutrients and proteins in the plasma. In some embodiments of the methods, compositions, and combinations disclosed herein, immunoglobulin is administered to the patient after a TPE session is completed. Without being bound by any theory, immunoglobulin can affect cell biomechanics in the direction of repair (Fuentealba et al., medRxiv, doi: doi.org/10.1101/2024.08.02.24310303 (2024)). In the combined TPE treatment, IV infusion and administration of oral supplements replenishes beneficial nutrients and proteins that have been removed during TPE and also provides additional nutrients and compounds that boost the immune system and promote regeneration. Surprisingly, although the methods, compositions, and combinations of detoxification disclosed herein utilizing combined TPE treatment or TPE only treatment were found to be effective, combined TPE was found to be superior at removing chemical toxins, heavy metals, and biotoxins as compared with TPE only treatment (Table 5 and Table 6). These results demonstrate the high capacity of the methods, compositions, and combinations of detoxification disclosed herein that utilize combined TPE for removing a wide range of toxins, which has not been reported for any currently available detoxification method.

As described herein, replacement fluid is required in TPE. In some embodiments, the replacement fluid is between 0.8 and 1.5 estimated plasma volume (EPV) with 5% albumin, saline, and anticoagulant. These parameters can be modified according to patient need and are well within the skill of the physician. The amount of albumin can be adjusted between 5-10%. Albumin can also be replaced with untreated fresh frozen human plasma or pathogen-inactivated (treated) human plasma. In some embodiments, the anticoagulant is citrate. Anticoagulants for use in the methods, compositions, and combinations of the disclosure can include, but are not limited to, heparin or citrate. In some embodiments, the TPE procedure is a centrifugation-based, continuous-flow process that uses a sterile fluid path with a single-use disposable kit. Patient height, weight, gender, and hematocrit are determined in order to estimate total blood volume and plasma volume for the patient. The physician's prescription provides the target fluid balance, the number of plasma volumes to replace, or the volume of replacement fluids. In the methods, compositions, and combinations of detoxification disclosed herein, TPE is performed using a suitable apheresis device. In general, the patient's blood is drawn using peripheral or central venous access into a sterile fluid path, single use disposable kit and is then centrifugally separated by the apheresis device into its cellular and plasma components. Before starting the procedure, the operator can perform automated custom prime using blood (if indicated). During the procedure, most of the plasma is removed while the remaining blood cells, as well as albumin and normal saline as replacement fluids, are returned to the patient. The process continues until the target plasma volume has been removed. During the procedure, the device uses weight scales and mechanical pumps to accurately track the waste plasma volume that has been removed from the patient and to monitor the volume of replacement fluid that has been returned. The separator device uses algorithms to adjust the plasma and replacement fluid pumps to achieve the desired volume exchange and to maintain the targeted fluid balance. When the targeted volume of the patient's plasma has been removed, the device prompts the operator to end the procedure and to reinfuse the patient's cells that remain in the kit.

### 3. IV INFUSION

In some embodiments, the methods, compositions, and combinations disclosed herein comprise a step of TPE followed by infusion of an intravenous (IV) composition and 2 to 3 minutes thereafter, administration of a glutathione solution. The goal of the IV infusion step is to provide an immediate supplement of nutrients and minerals that might be lost during TPE. In some embodiments, the components of the IV composition are as shown in Table 1 below. It is also contemplated that in some embodiments of the methods, compositions, and combinations disclosed herein, administration of the IV composition and glutathione solution are not required as the oral composition contains sufficient supplements and nutrients to achieve the same goal.

In some embodiments, the amount of IV composition being administered to the patient is between 300 mL and 350 mL. In certain embodiments, the amount of IV composition being administered to the patient is 326 mL. In some embodiments, the osmolality of the IV composition being administered to the patient is between 230 to 300 mOsm. The IV compositions for use in the methods, compositions, and combinations disclosed herein can be modified as described below. Glutathione is a strong antioxidant that would neutralize and react with the minerals, amino acids, and vitamins in the IV composition. Thus, it is important that the glutathione solution is administered only following 2 to 3 minutes after completion of the IV composition infusion. In some embodiments, the glutathione solution is administered to the patient at a dose of between 1 mg and 5000 mg. In certain embodiments, the glutathione solution is administered to the patient at a dose of 1000 mg. In some embodiments, a volume of between 3 mL to 10 mL of the glutathione solution is administered to the patient.

### 4. ADMINISTRATION OF ORAL COMPOSITION

In the methods, compositions, and combinations disclosed herein that utilize combined TPE, an oral composition is administered daily following TPE or following TPE in combination with IV infusion. The oral composition provides a slower and more sustained supplement of nutrients than IV infusion. In some embodiments, the components of the oral composition are as shown in Table 2 below. In some embodiments, the oral composition can be modified as discussed below. In some embodiments, a patient administers 30 g to 200 g of an oral composition comprising the supplements and nutrients shown in Table 4 once a day following TPE (with or without IV infusion) and continues to take the oral composition until all rounds of TPE and IV infusion (and follow up laboratory tests) have been completed. In some embodiments, a patient administers 60 g to 100 g of an oral composition comprising the supplements and nutrients shown in Table 4 once a day following TPE (with or without IV infusion) and continues to take the oral composition until all rounds of TPE and IV infusion (and follow up laboratory tests) have been completed. The amount of the oral composition to be administered by the patient may vary depending on the supplements and nutrients in the oral composition. It is contemplated that administration of the oral composition is continued until all rounds of TPE or TPE in combination with IV infusion have been completed, and in some embodiments, administration of the oral composition can continue even after completion of TPE or TPE in combination with IV infusion as a daily supplement to maintain patient well-being.

### 5. IV COMPOSITION

In some embodiments, an IV composition is used in combination with the methods, compositions, and combinations disclosed herein that utilize combined TPE. In certain embodiments, the IV composition comprises at least one mineral, at least one vitamin, and at least one amino acid. In some embodiments, the amount of IV composition being administered to the patient is between 300 mL and 350 mL. In certain embodiments, the amount of IV composition being administered to the patient is 326 mL. In some embodiments, the osmolality of the IV composition being administered to the patient is between 230 to 300 mOsm.

The selection of components and doses of those components for the IV compositions for use in the methods, compositions, and combinations disclosed herein is based on a number of factors, including mathematical modeling, which suggests that certain components offer significant replenishment after the removal of beneficial nutrients and molecules from the plasma as a result of TPE treatment. To formulate the IV and oral compositions for use in the methods, compositions, and combinations disclosed herein, several biological processes that are considered to be the most critical for healthy functions were first selected, including the mitochondrial metabolic pathway, anti-inflammatory pathway, antioxidant pathway, detoxifying pathway, and essential metabolic and enzymatic pathways. The components were then selected according to their functions and associations with these processes. Based on the assumption that selected biological processes are compromised as a result of long-term toxicity, the dose of each component is determined so that it is sufficient to improve biological functions but will also be safe for the patient. In some embodiments, the components of the IV composition are as shown in Table 3 below.

As used herein, the term “mineral” refers to inorganic elements or compounds that are essential for body functions. As used herein, the term “vitamin” refers to organic molecules that are essential in small quantities for proper metabolic function in the patient. Collectively, vitamins and minerals are termed “micronutrients.” They are typically not produced in sufficient quantity in the body and must therefore be obtained from the patient's diet. Minerals provide electrolytes and perform essential metabolic and blood functions in the body (Weyhn et al., Nutrients 14(3): 644 (2022); Ali, J. Trace Elem. Minerals 6: 100076 (2023)). Vitamins play a role in various important physiological processes (Reddy et al., “Biochemistry, Fat Soluble Vitamins”. Treasure Island (FL): StatPearls Publishing (2022)) and have been shown to maintain or enhance cognitive function in older adults (Vyas et al., Am. J. Clin. Nutr. 119(3): 692-701 (2024)).

In some embodiments, the at least one mineral in the IV composition for use in the methods, compositions, and combinations disclosed herein can include, but is not limited to, calcium, chromium, copper, iron, leucovorin, manganese, magnesium, molybdenum, potassium, selenium, sodium, or zinc. In some embodiments, the at least one mineral can be calcium chloride, calcium gluconate, chromium chloride, copper chloride, ferric gluconate complex, iron, iron sucrose, manganese chloride, magnesium chloride, magnesium sulfate, molybdenum, potassium, potassium chloride, selenium, sodium bicarbonate, zinc chloride, or zinc sulfate. The acceptable dose ranges of these minerals are provided in Table 1 below. In some embodiments, the minerals are calcium gluconate, magnesium chloride, molybdenum, potassium chloride, selenium, and zinc sulfate, and their doses in the IV composition are provided in Table 3.

In some embodiments, the at least one vitamin in the IV composition for use in the methods, compositions, and combinations disclosed herein can include, but is not limited to, ascorbic acid, biotin, folic acid, cobalamin, niacin, pyridoxine, riboflavin, or thiamine. In some embodiments, the at least one vitamin can be ascorbic acid, ascorbic acid corn-based and non-corn-based, biotin, calcium folinate, dexpanthenol, folate, hydroxocobalamin, methylcobalamin, niacin, niacinamide, pyridoxine, riboflavin, or thiamine. The acceptable dose ranges of these vitamins are provided in Table 1 below. In some embodiments, the vitamins are ascorbic acid, dexpanthenol, hydroxocobalamin, niacin, pyridoxine, riboflavin, and thiamine, and their doses in the IV composition are provided in Table 3 below.

As used herein, the term “amino acid” refers to an organic compound that contains both amino and carboxylic acid functional groups. Some amino acids are building blocks of proteins, while others play a role in some biological processes such as regulating gene expression, cell signaling, food intake, nutrient digestion and absorption, neurological development and behavior, energy metabolism, immunity, hormone secretion, and detoxification of substances (Wu, Encyclopedia of Human Nutrition (Fourth Edition), pp. 36-47 (Academic Press, ed: Benjamin Caballero, 2023); Dai et al., Adv. Exp. Med. Biol. 1354: 127-43 (2022)).

In some embodiments, the at least one amino acid in the IV composition for use in the methods, compositions, and combinations disclosed herein can include, but is not limited to, arginine, carnitine, glycine, lysine, or taurine. In some embodiments, the at least one amino acid can be arginine, L-carnitine, glycine, lysine, or taurine. The acceptable doses of these amino acids are provided in Table 1 below. In some embodiments, the amino acids are L-carnitine and taurine, and their doses are provided in Table 3 below.

In some embodiments, the IV composition for use in the methods, compositions, and combinations disclosed herein further comprises other components including, but not limited to, acetylcysteine, dexamethasone, dextrose, diphenhydramine, 2,3-dimercapto-1-propanesulfonic acid (DMPS), EDTA, famotidine, glutathione, nicotinamide adenine dinucleotide, or ondansetron. The acceptable doses of these components are provided in Table 1 below. In some embodiments, the other component is glutathione and its dose in the IV composition is provided in Table 3 below.

TABLE 1
Acceptable IV composition components and doses.

| | | | |
|---|---|---|---|
| | Min-max | | |
| Component | dose | Unit | Alternatives |

Minerals

| | | | |
|---|---|---|---|
| Calcium gluconate | 1-5000 | mg | Calcium chloride |
| Copper chloride | 0.1-2 | mg | |
| Iron | 1-250 | mg | Ferric gluconate complex, |
| | | | iron sucrose |
| Manganese | 0.05-0.5 | mg | |
| chloride | | | |
| Magnesium | 1-5000 | mg | Magnesium sulfate |
| chloride | | | |
| Molybdenum | 1-500 | mcg | |
| Potassium chloride | 1-40 | meq | |
| Selenium | 1-800 | mcg | |
| Zinc sulfate | 0.25-25 | mg | Zinc chloride |

Vitamins

| | | | |
|---|---|---|---|
| Ascorbic Acid | 1-25000 | mg | Corn-based or non-corn-based |
| Biotin | 1-500 | mg | |
| Calcium folinate | 1-5000 | mcg | |
| Dexpanthenol | 1-2000 | mg | |
| Folate | 1-50 | mg | |
| Hydroxocobalamin | 1-1000 | mg | Methylcobalamin, |
| | | | Cyanocobalamin |
| Niacin | 1-1000 | mg | Niacinamide |
| Pyridoxine | 1-1000 | mg | |
| Riboflavin | 1-100 | mg | |
| Thiamine | 1-1000 | mg | |

Amino Acids

| | | | |
|---|---|---|---|
| Alanine | 1-5000 | mg | |
| Arginine | 1-5000 | mg | |
| Aspartic Acid | 1-5000 | mg | |
| Carnitine | 1-5000 | mg | L-carnitine |
| Glutamic Acid | 1-5000 | mg | |
| Glycine | 1-5000 | mg | |
| Histidine | 1-5000 | mg | |
| Isoleucine | 1-5000 | mg | |
| Leucine | 1-5000 | mg | |
| Lysine | 1-5000 | mg | |
| Methionine | 1-5000 | mg | |
| Phenylalanine | 1-5000 | mg | |
| Proline | 1-5000 | mg | |
| Serine | 1-5000 | mg | |
| Taurine | 1-5000 | mg | |
| Threonine | 1-5000 | mg | |
| Tryptophan | 1-5000 | mg | |
| Tyrosine | 1-5000 | mg | |
| Valine | 1-5000 | mg | |

Other Components

| | | |
|---|---|---|
| Acetylcysteine | 1-3000 | mg |
| Dexamethasone | 1-25 | mg |
| Dextrose | 1-100 | mg |
| Diphenhydramine | 1-500 | mg |
| 2,3-dimercapto-1- | 1-500 | mg |
| propanesulfonic | | |
| acid (DMPS) | | |
| EDTA | 1-1000 | mg |
| Famotidine | 1-100 | mg |
| Glutathione | 1-5000 | mg |
| Nicotinamide | 1-1500 | mg |
| adenine | | |
| dinucleotide | | |
| Ondansetron | 1-40 | mg |

### 6. ORAL COMPOSITION

In some embodiments, an oral composition is used in combination with the methods, compositions, and combinations disclosed herein, wherein the oral composition is taken daily by a patient after completing the first round of TPE treatment. In some embodiments, the oral composition comprises at least one vitamin, at least one mineral, at least one anti-inflammatory compound, at least one antioxidant, at least one amino acid, at least one saccharide, and at least one herb. In some embodiments, the oral composition further comprises at least one protein to improve taste and flavoring. The rationale for the selection of the components and doses of those components in the oral composition is similar to that employed for the IV composition, as described herein. In some embodiments, the components of the oral composition are as shown in Table 4 below.

As used herein, the term “anti-inflammatory agent” refers to a compound or extract containing one or more compounds that have anti-inflammatory properties. The term “anti-inflammatory” refers to reduction of inflammation (redness, swelling, and pain) in the body. Anti-inflammatory agents block certain substances in the body that cause inflammation.

As used herein, the term “antioxidant” refers to a compound that inhibits oxidation, a chemical reaction that can produce free radicals. Antioxidants therefore protect the body against oxidative stress that is caused by free radicals or reactive oxidative species. As used herein, the term “saccharide” or “carbohydrate” refers to a compound consisting of carbon, hydrogen, and oxygen atoms. The term encompasses sugars, starch, and cellulose, which serve as energy stores in the body, and also includes fibers.

As used herein, the term “herb” refers to a plant, a plant part, or a plant extract that contains one or more compounds/ingredients that can be consumed and supplement the diet to provide health benefits. Many ingredients in herbs are anti-inflammatory agents, antioxidants, amino acids, saccharides, vitamins, and/or minerals that can exert their effects through one or more of the essential biological processes. Note that in many cases, a component in the oral composition or IV composition can have more than one function and belong to more than one group of components. For example, while resveratrol is an anti-inflammatory agent, it also has antioxidant properties.

In some embodiments, the at least one vitamin in the oral composition for use in the methods, compositions, and combinations disclosed herein can include, but is not limited to, biotin, citicoline, folate, inositol, cobalamin, niacin, pantothenic acid, pyridoxine, riboflavin, thiamine, vitamin A, vitamin B6, vitamin B12, vitamin C, vitamin D3, vitamin E, vitamin K1, or vitamin K2. In some embodiments, the at least one vitamin can be adenosylcobalamin, ascorbic acid, ascorbyl palmitate, benfotiamine, beta carotene, biotin, calcium pantothenate, cholecalciferol, choline (as choline bitartrate), citicoline (CDP-choline), D-alpha tocopheryl acetate, D-alpha tocopheryl succinate, folate (as folic acid), L-5-methyltetrahydrofolate calcium salt, L-5-methyltetrahydrofolate glucosamine salt, folinic acid, hydroxocobalamin, inositol, inositol hexanicotinate, inositol hexaphosphate, liposomal vitamin C, menaquinone-7, methylcobalamin, mixed carotenoids, mixed tocopherols, niacin, niacinamide, nicotinic acid, nicotinamide mononucleotide (NMN), nicotinamide riboside chloride (NR), para-aminobenzoic acid (PABA), pantesin H155 (pantethine 55%), pantothenic acid, pyridoxal 5 phosphate, pyridoxine HCl, pyridoxine, riboflavin, riboflavin 5 phosphate, thiamine, thiamine mononitrate, tocotrienols, sodium ascorbate, vitamin K1, or vitamin A palmitate. In some embodiments, the at least one vitamin can be adenosylcobalamin, ascorbic acid, ascorbyl palmitate, benfotiamine, beta carotene, biotin, calcium pantothenate, cholecalciferol, cyanocobalamin, D-alpha tocopheryl acetate, D-alpha tocopheryl succinate, folate, folinic acid, L-5-methyltetrahydrofolate calcium salt, L-5-methyltetrahydrofolate glucosamine salt, liposomal vitamin C, hydroxocobalamin, methylcobalamin, niacin, niacinamide, nicotinic acid, nicotinamide riboside chloride (NR), menaquinone-7, mixed carotenoids, mixed tocopherols, para-aminobenzoic acid (PABA), pantesin HF55 (pantethine 55%), pantothenic acid, pyridoxal 5 phosphate, pyridoxine, pyridoxine HCl, tocotrienols, riboflavin, riboflavin 5 phosphate, sodium ascorbate, thiamine, thiamine mononitrate, vitamin A palmitate, or vitamin K1. The acceptable doses of these vitamins are provided in Table 2 below. In some embodiments, the vitamins in the oral composition are ascorbic acid, biotin, cholecalciferol, L-5-methyltetrahydrofolate, glucosamine salt, methylcobalamin, niacin, nicotinamide riboside chloride (NR), pantothenic acid, pyridoxine, riboflavin, thiamine, vitamin A palmitate, and vitamin E as mixed tocopherols, and their doses are provided in Table 4 below.

In some embodiments, the at least one mineral in the oral composition for use in the methods, compositions, and combinations disclosed herein can include, but is not limited to, boron, calcium, chromium, copper, iodine, iron, lithium, magnesium, manganese, molybdenum, potassium, silicon, sodium, strontium, vanadium, or zinc. In some embodiments, the at least one mineral can be boron citrate, calcium BHB, calcium citrate, calcium D-glucarate, calcium glycinate, chromium polynicotinate, copper bisglycinate chelate, copper gluconate, dimagnesium malate, ferrous bisglycinate chelate, lithium orotate, L-selenomethionine), magnesium BHB, magnesium bisglycinate chelate buffered, magnesium bisglycinate fumaric acid, magnesium citrate, magnesium creatine chelate, magnesium glycinate glutamine chelate potassium chloride, magnesium L-threonate, magnesium taurinate, manganese glycinate, microcrystalline hydroxyapatite calcium, molybdenum glycinate chelate, potassium bicarbonate, potassium citrate, potassium iodide, potassium iodide, precipitated silicon dioxide, sea salt, strontium citrate, vanadyl sulfate, zinc bisglycinate chelate, zinc carnosine, zinc gluconate, zinc picolinate, or zinc sulfate. In some embodiments, the at least one mineral can be boron citrate, calcium citrate, calcium d-glucarate, calcium glycinate, chromium polynicotinate, copper bisglycinate chelate, copper gluconate, dimagnesium malate, ferrous bisglycinate chelate, lithium orotate, l-selenomethionine, magnesium bhb, magnesium bisglycinate chelate buffered, magnesium citrate, magnesium 1-threonate, magnesium taurinate, manganese glycinate, microcrystalline hydroxyapatite calcium, molybdenum glycinate chelate, potassium bicarbonate, potassium citrate, potassium iodide, precipitated silicon dioxide, sea salt, strontium citrate, vanadyl sulfate, zinc bisglycinate chelate, zinc carnosine, zinc gluconate, zinc picolinate, or zinc sulfate. The acceptable doses of these minerals are provided in Table 2 below. In some embodiments, the minerals in the oral composition are calcium D-glucarate, chromium polynicotinate, copper gluconate, magnesium bisglycinate chelate buffered, manganese glycinate, molybdenum glycinate chelate, potassium iodide, and zinc bisglycinate chelate, and their doses are provided in Table 4 below.

In some embodiments, the at least one amino acid in the oral composition for use in the methods, compositions, and combinations disclosed herein can include, but is not limited to, γ-amino butyric acid, alanine, arginine, asparagine, aspartic acid, carnitine, carnosine, citrulline, cysteine, glutamine, glutathione, glycine, histidine, isoleucine, leucine, lysine, methionine, ornithine, phenylalanine, proline, serine, theanine, threonine, tryptophan, tyrosine, or valine. In some embodiments, the at least one amino acid can be 5-hydroxytryptophan (5-HTP), acetyl L-carnitine HCl, alpha ketoglutaric acid (AKG), calcium beta-hydroxy-beta-methylbutyrate (calcium HMB), DL-phenylalanine, L-alanine, L-arginine, L-arginine HCl, L-asparagine monohydrate, L-aspartic acid, L-carnitine tartrate, L-carnosine, L-citrulline, L-glutamine, L-glutathione (reduced), L-glycine, L-histidine HCl, L-isoleucine, L-leucine, L-lysine, L-lysine HCl, L-methionine, L-ornithine, L-ornithine HCl, L-phenylalanine, L-proline, L-serine, L-theanine, L-threonine, L-tryptophan, L-tyrosine, L-valine, pharma γ-amino butyric acid (PharmaGABA), phosphatidylserine, S-adenosyl-L-methionine (sam-e), taurine, trimethyl glycine (TMG)-betaine anhydrous, or γ-amino butyric acid (GABA). The acceptable doses of these amino acids are provided in Table 2 below. In some embodiments, the amino acids in the oral composition are L-alanine, L-arginine HCl, L-glutamine, L-glycine, L-lysine HCl, L-methionine, L-ornithine HCl, L-proline, L-tyrosine, taurine, and Trimethyl Glycine (TMG)-Betaine Anhydrous, and their doses are provided in Table 4 below. In some embodiments, the oral composition in Table 4 further comprises 100 mg of phosphatidylserine and 100 mg of L-theanine.

In some embodiments, the at least one anti-inflammatory agent in the oral composition for use in the methods, compositions, and combinations disclosed herein can include, but is not limited to, bromelain 2400 gelatin dissolving units (GDU), celery seed extract, elderberry extract 4:1, glucosamine sulfate potassium, Gymnema sylvestre leaf extract with 25% gymnemic acid, holy basil extract 2%, huperzine, noni fruit extract 4:1, omega-3 comprising 2:1 eicosapentaenoic acid (epa):docosahexaenoic acid (dha)pea (palmitoylethanolamide), omega-3 comprising 3:1 eicosapentaenoic acid (epa):docosahexaenoic acid (dha), pterostilbene, Pueraria lobata root extract with 40% isoflavones, resveratrol root extract, skullcap root extract with 30% baicalin, superoxide dismutase 14000 iu/g, Unicaria tomentosa bark extract with 3% oxindole alkaloids, Urtica dioica L. root extract 4:1, witch hazel extract, or yucca root extract. The acceptable doses of these anti-inflammatory compounds are provided in Table 2 below. In some embodiments, the anti-inflammatory agents in the oral composition are bromelain 2400 gelatin dissolving units (GDU), pterostilbene, resveratrol root extract, skullcap root extract with 30% baicalin), superoxide dismutase 14000 IU/g, Unicaria tomentosa Bark Extract (3% Oxindole Alkaloids), Omega-3 comprising 2:1 eicosapentaenoic acid (EPA):docosahexaenoic acid (DHA), and Noni Fruit Extract 4:1, and their doses are provided in Table 4 below.

In some embodiments, the at least one antioxidant in the oral composition for use in the methods, compositions, and combinations disclosed herein can include, but is not limited to, Alpha Lipoic Acid, Artemisinin, Artemether, Artichoke Flower Extract (5% Polyphenols), Asimina triloba, Astaxanthin, Astragalus extract, Bioflavonoid Complex from Citrus lemon with 90% Bioflavonoids, Blueberry extract powder, Boswellia serrata extract, Broccoli Seed Extract (13% Glucoraphanin), Camellia sinensis Leaf Extract with 50% Epigallocatechin gallate (EGCG), Camu-Camu with 20% Vitamin C, Capsicum, Coenzyme Q10, Coenzyme Q10 blend from Sunflower Lecithin, Crataugus pinnatifida Bge. Berry Extract 10:1, Curcuma longa Extract, Dandelion Root Extract, Dichrostachys glomerata Powder, Diindolylmethane (DIM), Ellagic Extract 90%, Genistein 98%, Ginkgo biloba Leaf Extract with 24% Ginkoflavonglycosides/6% Terpene Lactones, Goldenrod Extract Powder 4:1, Gotu Kola, Grape Seed Extract, Grapefruit seed extract, Green Coffee Bean Extract 50%, Hibiscus Flower Extract 5%, Hops Extract, Indole-3-Carbinol (I3C), L-Glutathione, Linum sitatissimum Seed, Lutein, Luteolin, Lycopene tomato extract, Lumefantrine, Micro pyrroloquinoline quinone (PQQ), Milk Thistle Seed extract (80% Silymarin), Monk Fruit Extract, Moringa Leaf Powder, N-Acetyl-L-Cysteine (NAC), Olea europeae Leaf Extract with 18% Oleuropein, Organic Pineapple Fruit Powder, Origanum vulgare Leaf Extract 10:1, Oxaloacetate, Punica granatum Fruit Extract (30% Punicalagins), Quercetin, Rosemary Extract, Schisandra Berry Extract, Sea Cucumber, Shilajit Extract, Standardized extract from the Asparagus officinalis stem, Tart Cherry Powder, Uvae ursi Extract, Vaccinium myrtillus Fruit Extract with 25% Anthocyanins, or Vitis vinfera Seed Extract with 95% Proanthocyanidins. The acceptable doses of these antioxidants are provided in Table 2 below. In some embodiments, the antioxidants in the oral composition are Alpha Lipoic Acid, Artichoke Flower Extract (5% Polyphenols), Astaxanthin, Boswellia serrata extract, Broccoli Seed Extract (13% Glucoraphanin), Coenzyme Q10 blend from Sunflower Lecithin, Dandelion Root Extract, Grape Seed Extract, Camellia sinensis Leaf Extract with 50% Epigallocatechin gallate (EGCG), Hops Extract, L-Glutathione, Micro PQQ, Milk Thistle Seed extract (80% Silymarin), N-Acetyl-L-Cysteine (NAC), Punica granatum Fruit Extract (30% Punicalagins), Quercetin, Rosemary Extract, Schisandra Berry Extract, Shilajit Extract, and Curcuma longa Extract, and their doses are provided in Table 4 below. In some embodiments, the antioxidants in Table 4 further comprise 120 mg of Ginkgo biloba Leaf Extract with 24% Ginkoflavonglycosides/6% Terpene Lactones. In some embodiments, the antioxidants in Table 4 further comprise 1500 mg sea cucumber, 360 mg of artemisinin, 3 mg Capsicum, 1000 mg of oxaloacetate, 4000 mg of Curcuma longa Extract, 250 mg genistein, 30 mg Asimina triloba, 50 mg lycopene, 1000 mg Theracurmin™ HP, and 1 capsule of Artemax™ Plus containing 20 mg artemether and 120 mg lumefantrine

In some embodiments, the at least one saccharide in the oral composition for use in the methods, compositions, and combinations disclosed herein can include, but is not limited to, 1,3 Beta Glucan 85%, D-Ribose, Fucoidan, Larch Arabinogalactan Powder, LeptiCore®, or organic coconut sugar. The acceptable doses of these saccharides are provided in Table 2 below. In some embodiments, the saccharides in the oral composition are 1,3 Beta Glucan 85%, D-Ribose, and Larch Arabinogalactan Powder, and their doses are provided in Table 4 below.

In some embodiments, the at least one herb in the oral composition for use in the methods, compositions, and combinations disclosed herein can include, but is not limited to, Acanthopanax senticous Stem and Root Extract with 0.8% Eleutherosides, Agaricus Extract, Allium sativum Bulb Extract with 1% Allicin, Aloe Vera Gel, Amla Powder, AnaGain Nu, Andrographis paniculata extract, Apigenin 98%, Apple Extract 30% Polyphenols, Arjuna 10:1, Artemisia absinthium extract, Bacopa Monnieri Extract, Beet Root Extract, Berberis aquifolium Root Extract 4:1, Berberis aristata root extract with 97% Berberine HCl, Bitter Melon Extract, Black Cohosh Extract, Black Walnut Hull Extract 10:1, Buchu Leaf Powder, Burdock Root Extract, California Poppy, Chaga Gold, Chamomile, Chaste Tree Berry Extract, Chlorella vulgaris Whole Plant, Cinnamonum burmannii Bark Extract with 3% trimeric and tetrameric Type-A Polymers, Cissus quadrangularis extract, Citrus bergamia with 40% Polyphenol Fraction, Citrus sinensis L. & palm Elaeis guineensis extract, Clove Extract Powder 4:1, Coconut Powder 70%, Coleus forskohlii Extract 10%, Cordyceps sinensis Extract with 8% Cordycepic Acid, Danshen (Red Sage), Echinacea, Epimedium Extract 8:1, Evodiamine, Fenugreek Extract with 50% Saponins UV, Ginger rhizome Extract (5% Gingerols), Goat's Rue, Hericium erinaceus extract, Hyssop Herb Powder, Kudzu root, Lagerstroemia speciosa L. Leaf Extract with 1% Corosolic Acid, Lemon Balm Extract, Maca Extract 10:1, Magnolia Bark Extract with 2% Honokiol, Magnolia Bark Extract with 90% Honokiol, Maitake Mushroom (TD-Fraction), Marshmallow Root, Melatonin, Naringin Extract, Nigella sativa Powder, Organic Calendula Flower Powder, Organic Coconut Cream Powder, Parsley Leaf Powder, Passiflora incarnata L. Aerial Powder, Piper nigrum fruit extract standardized to 95%, Psyllium Husk Powder, Red Clover Extract, Rhodiola rosea Root Extract with 4% Rosavins and 1% Salidroside, Sage, Saffron Extract with 0.3% Safranal, or Withania somnifera Root Extract. The acceptable doses of these herbs are provided in Table 2 below. In some embodiments, the herbs in the oral composition are Ginger rhizome extract with 5% gingerols and bitter melon extract, and their doses are provided in Table 4 below. In some embodiments, the herbs in the oral composition (as set forth in Table 4) further comprise 100 mg of Hericium erinaceus extract, 100 mg of Withania somnifera Root Extract, and 100 mg of Bacopa Monnieri Extract. In some embodiments, the herbs in the oral composition (as set forth in Table 4) further comprise 1800 mg Agaricus extract, 20 mg of melatonin, and 400 mg Allium sativum.

In some embodiments, the oral composition further comprises at least one protein to increase flavoring and taste. In some embodiments, the at least one protein in the oral composition is rice protein and its acceptable dose is provided in Table 2 below. In other embodiments, the protein in the oral composition is rice protein and its dose is provided in Table 4 below. In other embodiments, the protein is a powdered form of protein from plants (e.g., soybeans, peas, rice, potatoes, or hemp), eggs, or milk (casein or whey protein), and the protein is administered at a dose of between 1000 and 30000 mg.

In some embodiments, the oral composition further comprises other components including, but not limited to, Acacia Fiber, Adrenal Concentrate, Amylase (Food Grade), Apple Cider Vinegar, Bacillus clausii (100 Billion CFU/G), Bacillus coagulans (200 Billion CFU/G), Bacillus mesentericus (100 Billion CFU/G), Bacillus subtilis (100 Billion CFU/G), Betaine HCl, Bioferrin, Bovine Thyroid Tissue Powder, Pure Mango Fruit Powder, Catalase, Choline Bitartrate, Chondroitin Sulfate, Citric Acid, Creatine Monohydrate, Dehydroepiandrosterone (DHEA), Fisetin 98%, Gamma Oryzanol 97%, Guar Gum, Gugulipid Extract, Horsetail 7% Silica, Hyaluronic Acid, Hydrolyzed Collagen, Peach Ceramides, Lactase Powder, Lavender Extract, Lecithin (sunflower), Fenugreek Extract, Glycyrrhiza glabra Root Extract with 20% Glycyrrhizic Acid, Malic Acid, Methylsulfonylmethane (MSM), Mucuna pruriens with 98% 3,4-dihydroxy phenylalanine (L-Dopa), Muira puama Root Extract 10:1, Nattokinase (2,000 FU/100 mg), Natural Caffeine 98% (From Green Tea), Panax ginseng 5% Powdered Extract, Tabebuia impetiginosa inner bark powder, PeakO2® mushroom complex, Phosphatidylcholine (from Sunflower Lecithin), Pine Bark Extract, Plant Sterols with 95% Phytosterols, Policosanol 95%, Porcine Kidney Powder, Pregnenolone, Pueraria mirifica Root Powder, Red Yeast Rice Powder, Reishi Mushroom Powder, Magnolia officinalis and Phellodendron amurense blend extract, Rhubarb (Rheum) Root Extract 10:1, Serenoa serrulata Fruit Extract with 45% Total Fatty Acids, Shatavari Extract, Shiitake, Wheat Germ Extract, Spirulina, Stevia extract, Strawberry Flavor Blend, Strawberry Fruit Powder, Eurycoma longlifolia Root Extract, Tribulis terrestris L. Fruit Extract with 40% Saponins, Turkey Tail, Undenatured type II collagen (UC-II), Valerian Extract with 0.8% valerenic acids, Vinpocetine 99%, Wild Lettuce Powder, Willow Bark Dry Extract, Verisol® collagen, or Zeaxanthine. The acceptable doses of the other components are provided in Table 2 below. In some embodiments, the oral composition in Table 4 further comprises 30 mg vinpocetine and 100 mg of Panax ginseng 5% Powdered Extract.

TABLE 2
Acceptable oral composition components and doses.

| | | | |
|---|---|---|---|
| Component | Min-max doses | Units | Alternatives |

Minerals

| | | | |
|---|---|---|---|
| Boron citrate | 1-10,000 | mcg | |
| Calcium citrate | 20-1200 | mg | |
| Calcium D-glucarate | 25-1000 | mg | |
| Calcium glycinate | 10-2000 | mg | |
| Chromium polynicotinate | 10-1500 | mcg | |
| Copper bisglycinate chelate | 0.1-8 | mg | |
| Copper gluconate | 0.1-2000 | mg | |
| Dimagnesium malate | 10-2000 | mg | |
| Ferrous bisglycinate chelate | 1-100 | mcg | |
| Lithium orotate | 5-50,000 | mcg | |
| L-selenomethionine | 2-8000 | mg | |
| Magnesium bisglycinate chelate | 5-11,000 | mg | |
| buffered | | | |
| Magnesium citrate | 5-1000 | mg | Magnesium BHB, magnesium |
| | | | L-threonate |
| Magnesium taurinate | 5-10000 | mg | |
| Manganese glycinate | 0.5-80 | mg | |
| Microcrystalline hydroxyapatite | 10-1500 | mg | |
| calcium | | | |
| Molybdenum glycinate chelate | 1-2000 | mcg | |
| Potassium bicarbonate | 1-2000 | mg | |
| Potassium citrate | 5-2000 | mg | |
| Potassium iodide | 10-25,000 | mcg | |
| Precipipated silicon dioxide | 1-5000 | mg | |
| Sea salt | 1-2500 | mg | |
| Strontium citrate | 5-1000 | mg | |
| Vanadyl sulfate | 1-10,000 | mcg | |
| Zinc bisglycinate chelate | 1-200 | mg | |
| Zinc carnosine | 5-150 | mg | |
| Zinc gluconate | 1-200 | mg | |
| Zinc picolinate | 1-100 | mg | |
| Zinc sulfate | 1-100,000 | mg | |

Vitamins

| | | | |
|---|---|---|---|
| Adenosylcobalamin | 10-5000 | mcg | Hydroxycobalamin, |
| | | | Cyanocobalamin |
| Ascorbic Acid | 1-10,000 | mg | |
| Ascorbyl palmitate | 10-5000 | mg | |
| Benfotiamine | 1-600 | mg | |
| Biotin | 15-25000 | mcg | |
| Calcium pantothenate | 1-3000 | mg | |
| Cholecalciferol | 10-100,000 | IU | |
| D-alpha tocopheryl acetate | 8-2000 | IU | |
| D-alpha tocopheryl succinate | 10-2000 | IU | |
| Folate | 1-5000 | mcg | |
| Folinic acid | 10-5000 | mcg | |
| L-5-methyltetrahydrofolate, | 5-15000 | mcg | |
| calcium salt | | | |
| L-5-Methyltetrahydrofolate, | 10-15,000 | mcg | |
| glucosamine salt | | | |
| Liposomal vitamin C | 1-4000 | mg | |
| Menaquinone-7 (Vitamin K2) | 1-1000 | mcg | |
| Methylcobalamin | 10-10,000 | mcg | |
| Niacin | 0.1-1500 | mg | Niacinamide |
| Nicotinamide Riboside Chloride | 1-1000 | mg | |
| (NR) | | | |
| Nicotinic acid | 5-1000 | mg | |
| Nicotinamide Mononucleotide | 250-1200 | mg | |
| Pantesin HF55 (Pantethine 55%) | 1-1000 | mg | |
| Pantothenic Acid | 1-1500 | mg | |
| Para-aminobenzoic acid (PABA) | 5-1000 | mg | |
| Pyridoxal-5-phosphate | 0.1-600 | mg | |
| Pyridoxine | 1-300 | mg | |
| Pyridoxine HC1 | 1-600 | mg | |
| Riboflavin | 1-500 | mg | |
| Riboflavin-5-phosphate | 1-1100 | mg | |
| Sodium ascorbate | 10-10,000 | mg | |
| Thiamine | 1-300 | mg | |
| Thiamine mononitrate | 1-535 | mg | |
| Tocotrienols | 1-10,000 | mg | |
| Vitamin A Palmitate | 1-2000 | mcg | |
| Vitamin E as Mixed Tocopherols | 10-200 | IU | |
| Vitamin K1 | 10-2600 | mcg | |

Amino Acids

| | | |
|---|---|---|
| 5-hydroxytryptophan (5-HTP) | 10-700 | mg |
| Acetyl L-Carnitine HCI | 5-2500 | mg |
| Alpha ketoglutaric acid (AKG) | 1-10,000 | mg |
| Calcium beta-hydroxy-beta- | 1-100,000 | mg |
| methylbutyrate (calcium HMB) | | |
| DL-Phenylalanine | 1-2000 | mg |
| L-Alanine | 1-5000 | mg |
| L-Arginine HCI | 1-5000 | mg |
| L-Asparagine monohydrate | 1-5000 | mg |
| L-Aspartic acid | 1-5000 | mg |
| L-Carnitine Tartrate | 1-5000 | mg |
| L-Carnosine | 10-1000 | mg |
| L-Citrulline | 10-6000 | mg |
| L-Glutamine | 1-30,000 | mg |
| L-Glycine | 1-6000 | mg |
| L-Histidine HC1 | 1-5000 | mg |
| L-Isoleucine | 1-5000 | mg |
| L-Leucine | 1-5000 | mg |
| L-Lysine HC1 | 5-7000 | mg |
| L-Methionine | 1-5000 | mg |
| L-Ornithine HC1 | 1-2000 | mg |
| L-Phenylalanine | 1-5000 | mg |
| L-Proline | 1-5000 | mg |
| L-Serine | 1-5000 | mg |
| L-Theanine | 5-2500 | mg |
| L-Threonine | 1-5000 | mg |
| L-Tryptophan | 1-5000 | mg |
| L-Tyrosine | 1-5000 | mg |
| L-Valine | 1-5000 | mg |
| Pharma γ-amino butyric acid | 5-1000 | mg |
| (PharmaGABA) | | |
| Phosphatidylserine | 10-2000 | mg |
| S-adenosyl-L-methionine (SAM- | 1-2000 | mg |
| e) | | |
| Taurine | 1-24,000 | mg |
| Trimethyl Glycine (TMG) - | 10-1500 | mg |
| Betaine Anhydrous | | |
| γ-amino butyric acid (GABA) | 5-1500 | mg |

Anti-inflammatory agents

| | | | |
|---|---|---|---|
| Bromelain 2400 gelatin | 35-1000 | mg | |
| dissolving units (GDU) | | | |
| Celery seed extract | 25-1000 | mg | |
| Elderberry extract 4:1 | 1-2000 | mg | |
| Glucosamine sulfate potassium | 50-2500 | mg | |
| Gymnema Sylvestre leaf extract | 25-1000 | mg | |
| with 25% gymnemic acid | | | |
| Holy basil extract 2% | 1-3000 | mg | |
| Huperzine | 5-500 | mcg | |
| Noni Fruit Extract 4:1 | 1-2000 | mg | Mouse's Pineapple, Yellow |
| | | | root, jumbie breadfruit |
| Omega-3 comprising 2:1 | 25-3000 | mg | Omega-3 comprising 3:1 |
| eicosapentaenoic acid | | | eicosapentaenoic acid |
| (EPA):docosahexaenoic acid | | | (EPA):docosahexaenoic acid |
| (DHA) | | | (DHA) |
| Pterostilbene | 10-250 | mg | |
| Pueraria Lobata root extract with | 50-1000 | mg | |
| 40% isoflavones | | | |
| Resveratrol Root Extract | 5-2000 | mg | |
| Skullcap Root Extract (30% | 25-1000 | mg | |
| Baicalin) | | | |
| Superoxide dismutase 14000 IU/g | 1-2000 | mg | |
| Unicaria Tomentosa Bark Extract | 50-1200 | mg | |
| (3% Oxindole Alkaloids) | | | |
| Urtica Dioica L. root extract 4:1 | 50-1000 | mg | |
| Witch hazel extract | 50-500 | mg | |
| Yucca root extract | 5-1000 | mg | |

Antioxidants

| | | | |
|---|---|---|---|
| Alpha Lipoic Acid | 5-1800 | mg | |
| Artemisinin | 100-1500 | mg | |
| Artemether 20 mg and | 1-6 | tablet | ArteMax ™ Plus |
| Lumefantrine 120 mg in a tablet | | | |
| Artichoke Flower Extract (5% | 20-1000 | mg | |
| Polyphenols) | | | |
| Asimina Triloba | 1-100 | mg | |
| Astaxanthin | 1-20 | mg | |
| Astragalus extract | 20-2000 | mg | |
| Bioflavonoid Complex from | 10-2000 | mg | |
| Citrus lemon with 90% | | | |
| Bioflavonoids | | | |
| Blueberry extract powder | 5-1000 | mg | |
| Boswellia Serrata extract | 5-7000 | mg | |
| Broccoli Seed Extract (13% | 20-1000 | mg | |
| Glucoraphanin) | | | |
| Camellia Sinensis Leaf Extract | 10-1500 | mg | |
| with 50% Epigallocatechin | | | |
| gallate (EGCG) | | | |
| Camu-Camu with 20% Vitamin C | 5-4000 | mg | |
| Capsicum | 1-20 | mg | |
| Coenzyme Q10 | 5-2000 | mg | |
| Coenzyme Q10 blend from | 10-1000 | mg | |
| Sunflower Lecithin | | | |
| Crataugus pinnatifida Bge. Berry | 50-1500 | mg | |
| Extract 10:1 | | | |
| Curcuma Longa Extract | 50-8000 | mg | Theracurmin ™ HP |
| Dandelion Root Extract | 20-1000 | mg | |
| Dichrostachys Glomerata Powder | 10-500 | mg | |
| Diindolylmethane (DIM) | 10-450 | mg | |
| Ellagic Extract 90% | 10-1000 | mg | |
| Genistein 98% | 1-2000 | mg | |
| Ginkgo Biloba Leaf Extract with | 80-2000 | mg | |
| 24% Ginkoflavonglycosides/6% | | | |
| Terpene Lactones | | | |
| Goldenrod Extract Powder 4:1 | 10-1000 | mg | |
| Gotu Kola | 50-1000 | mg | |
| Grape Seed Extract | 5-1000 | mg | |
| Grapefruit seed extract | 25-1000 | mg | |
| Green Coffee Bean Extract 50% | 5-2000 | mg | |
| Hibiscus Flower Extract 5% | 1-2000 | mg | |
| Hops Extract | 20-1000 | mg | |
| Indole-3-Carbinol (I3C) | 5-1000 | mg | |
| L-Glutathione | 50-1000 | mg | |
| Linum Usitatissimum Seed | 50-2000 | mg | |
| Lutein | 100-40,000 | mcg | |
| Luteolin | 1-1000 | mg | |
| Lycopene tomato extract | 1-500 | mg | |
| Micro PQQ | 1-800 | mg | |
| Milk Thistle Seed extract (80% | 30-2400 | mg | |
| Silymarin) | | | |
| Monk Fruit Extract | 5-5000 | mg | |
| Moringa Leaf Powder | 5-2000 | mg | |
| N-Acetyl-L-Cysteine (NAC) | 5-5000 | mg | |
| Olea Europeae Leaf Extract with | 30-1000 | mg | |
| 18% Oleuropein | | | |
| Organic Pineapple Fruit Powder | 1-100,000 | mg | |
| Origanum vulgare Leaf Extract | 20-1000 | mg | |
| 10:1 | | | |
| Oxaloacetate | 100-5000 | mg | |
| Punica granatum Fruit Extract | 10-2000 | mg | |
| (30% Punicalagins) | | | |
| Quercetin | 10-2000 | mg | |
| Rosemary Extract | 5-1000 | mg | |
| Schisandra Berry Extract | 20-3000 | mg | |
| Sea cucumber | 100-5000 | mg | |
| Shilajit Extract | 20-1500 | mg | |
| Standardized extract from the | 1-2000 | mg | |
| Asparagus Officinalis stem | | | |
| Tart Cherry Powder | 25-3000 | mg | |
| Uvae Ursi Extract | 50-2000 | mg | |
| Vaccinium myrtillus Fruit Extract | 50-1000 | mg | |
| with 25% Anthocyanins | | | |
| Vitis Vinifera Seed Extract with | 5-1000 | mg | |
| 95% Proanthocyanidins | | | |

Saccharides

| | | |
|---|---|---|
| 1,3 Beta Glucan 85% | 7.5-1000 | mg |
| D-Ribose | 50-5000 | mg |
| Fucoidan | 10-1000 | mg |
| Larch Arabinogalactan Powder | 50-2000 | mg |
| LeptiCore ® | 300-2000 | mg |
| Organic Coconut Sugar | 1-5000 | mg |

Herbs

| | | |
|---|---|---|
| Acanthopanax senticous Stem | 25-750 | mg |
| and Root Extract with 0.8% | | |
| Eleutherosides | | |
| Agaricus Extract | 100-5400 | mg |
| Allium sativum Bulb Extract with | 100-4000 | mg |
| 1% Allicin | | |
| Aloe Vera Gel | 25-500 | mg |
| Amla Powder | 1-100,000 | mg |
| AnaGain Nu | 1-2000 | mg |
| Andrographis paniculata extract | 0.5-2000 | mg |
| Apigenin 98% | 0.5-100,000 | mg |
| Apple Extract 30% Polyphenols | 1-2000 | mg |
| Arjuna 10:1 | 1-2000 | mg |
| Artemisia Absinthium extract | 1-2000 | mg |
| Bacopa Monnieri Extract | 50-500 | mg |
| Beet Root Extract | 1-2000 | mg |
| Berberis aquifolium Root Extract | 10-1000 | mg |
| 4:1 | | |
| Berberis aristata root extract with | 20-2000 | mg |
| 97% Berberine HCl | | |
| Bitter Melon Extract | 50-3000 | mg |
| Black Cohosh Extract | 10-300 | mg |
| Black Walnut Hull Extract 10:1 | 1-100,000 | mg |
| Buchu Leaf Powder | 5-500 | mg |
| Burdock Root Extract | 50-1000 | mg |
| California Poppy | 5-180 | mg |
| Chaga Gold | 1-1000 | mg |
| Chamomile | 10-1000 | mg |
| Chaste Tree Berry Extract | 10-1000 | mg |
| Chlorella vulgaris Whole Plant | 50-3000 | mg |
| Cinnamonum burmannii Bark | 25-1000 | mg |
| Extract with 3% trimeric and | | |
| tetrameric Type-A Polymers | | |
| Cissus Quadrangularis extract | 100-500 | mg |
| Citrus bergamia with 40% | 1-2600 | mg |
| Polyphenol Fraction | | |
| Citrus sinensis L. & palm Elaeis | 10-1000 | mg |
| guineensis extract | | |
| Clove Extract Powder 4:1 | 5-5000 | mg |
| Coconut Powder 70% | 5-5000 | mg |
| Coleus Forskohlii Extract 10% | 20-1000 | mg |
| Cordyceps sinensis Extract with | 50-3000 | mg |
| 8% Cordycepic Acid | | |
| Danshen (Red Sage) | 1-2000 | mg |
| Echinacea | 1-2000 | mg |
| Epimedium Extract 8:1 | 0.5-2000 | mg |
| Evodiamine | 5-200 | mg |
| Fenugreek Extract with 50% | 1-5000 | mg |
| Saponins UV | | |
| Ginger rhizome Extract (5% | 10-1200 | mg |
| Gingerols) | | |
| Goat's Rue | 50-500 | mg |
| Hericium Erinaceus extract | 1-3000 | mg |
| Hyssop Herb Powder | 1-2000 | mg |
| Kudzu Root | 50-1000 | mg |
| Lagerstroemia speciosa L. Leaf | 5-200 | mg |
| Extract with 1% Corosolic Acid | | |
| Lemon Balm Extract | 1-5000 | mg |
| Maca Extract 10:1 | 1-2000 | mg |
| Magnolia Bark Extract with 2% | 1-1000 | mg |
| Honokiol | | |
| Magnolia Bark Extract with 90% | 5-500 | mg |
| Honokiol | | |
| Maitake Mushroom (TD- | 10-500 | mg |
| Fraction) | | |
| Marshmallow Root | 50-500 | mg |
| Melatonin | 0.25-60 | mg |
| Naringin Extract | 1-2000 | mg |
| Nigella sativa Powder | 1-2800 | mg |
| Organic Calendula Flower | 1-2000 | mg |
| Powder | | |
| Organic Coconut Cream Powder | 1-5000 | mg |
| Parsley Leaf Powder | 5-2000 | mg |
| Passiflora incarnata L. Aerial | 10-1000 | mg |
| Powder | | |
| Piper nigrum fruit extract | 1-50 | mg |
| standardized to 95% | | |
| Psyllium Husk Powder | 50-7000 | mg |
| Red Clover Extract | 5-1000 | mg |
| Rhodiola rosea Root Extract with | 9-2000 | mg |
| 4% Rosavins and 1% Salidroside | | |
| Sage | 5-250 | mg |
| Saffron Extract with 0.3% | 1-2000 | mg |
| Safranal | | |
| Withania somnifera Root Extract | 50-2000 | mg |

Proteins

| | | |
|---|---|---|
| Rice Protein | 1000-30000 | mg |

Other Components

| | | |
|---|---|---|
| Acacia Fiber | 1-2000 | mg |
| Adrenal Concentrate | 20-1000 | mg |
| Amylase (Food Grade) | 1-300 | IU |
| Apple Cider Vinegar | 1-1000 | mg |
| Bacillus Clausii (100 billion | 1-200 | mg |
| CFU/G) | | |
| Bacillus Coagulans (200 billion | 1-200 | mg |
| CFU/G) | | |
| Bacillus Mesentericus (100 | 1-200 | mg |
| billion CFU/G) | | |
| Bacillus Subtilis (100 billion | 1-200 | mg |
| CFU/G) | | |
| Betaine HCl | 5-2000 | mg |
| Bioferrin | 1-2000 | mg |
| Bovine Thyroid Tissue Powder | 1-2000 | mg |
| Catalase | 5-1000 | mg |
| Choline Bitartrate | 5-1500 | mg |
| Chondroitin Sulfate | 50-2000 | mg |
| Citric Acid | 10-2500 | mg |
| Creatine Monohydrate | 50-10,000 | mg |
| Dehydroepiandrosterone (DHEA) | 1-250 | |
| Eurycoma longlifolia Root | 25-2000 | mg |
| Extract | | |
| Fenugreek Extract | 1-2000 | mg |
| Fisetin 98% | 1-2000 | mg |
| Gamma Oryzanol 97% | 10-500 | mg |
| Glycyrrhiza glabra Root Extract | 10-500 | mg |
| with 20% Glycyrrhizic Acid | | |
| Guar Gum | 10-1000 | mg |
| Gugulipid Extract | 20-1500 | mg |
| Horsetail 7% Silica | 1-1000 | mg |
| Hyaluronic Acid | 1-500 | mg |
| Hydrolyzed Collagen | 5-20,000 | mg |
| Peach Ceramides | 1-2000 | mg |
| Lactase Powder | 1-500 | mg |
| Lavender Extract | 1-5000 | mg |
| Lecithin (sunflower) | 20-10,000 | mg |
| Magnolia officinalis and | 67-750 | mg |
| Phellodendron amurense blend | | |
| extract | | |
| Malic Acid | 1-2400 | mg |
| Methylsulfonylmethane (MSM) | 30-5000 | mg |
| Mucuna Pruriens with 98% 3,4- | 20-1000 | mg |
| dihydroxy phenylalanine (L- | | |
| Dopa) | | |
| Muira Puama Root Extract 10:1 | 1-2000 | mg |
| Nattokinase (2,000 FU/100mg) | 1-100,000 | mg |
| Natural Caffeine 98% (From | 10-300 | mg |
| Green Tea) | | |
| Panax Ginseng 5% Powdered | 1-2000 | mg |
| Extract | | |
| PeakO2 ® mushroom complex | 1-2000 | mg |
| Phosphatidylcholine (from | 5-3000 | mg |
| Sunflower Lecithin) | | |
| Pine Bark Extract | 1-1000 | mg |
| Plant Sterols with 95% | 25-3000 | mg |
| Phytosterols | | |
| Policosanol 95% | 1-1000 | mg |
| Porcine Kidney Powder | 1-2000 | mg |
| Pregnenolone | 5-300 | mg |
| Pueraria mirifica Root Powder | 1-2000 | mg |
| Pure Mango Fruit Powder | 1-1000 | mg |
| Red Yeast Rice Powder | 50-5000 | mg |
| Reishi Mushroom Powder | 10-3000 | mg |
| Rhubarb (Rheum) Root Extract | 1-1000 | mg |
| 10:1 | | |
| Serenoa serrulata Fruit Extract | 50-1500 | mg |
| with 45% Total Fatty Acids | | |
| Shatavari Extract | 10-1000 | mg |
| Shiitake | 10-1000 | mg |
| Spirulina | 0.1-2000 | mg |
| Stevia extract | 5-2000 | mg |
| Strawberry Flavor Blend | 5-3700 | mg |
| Strawberry Fruit Powder | 1-2000 | mg |
| Tabebuia impetiginosa inner bark | 5-2000 | mg |
| powder | | |
| Tribulis terrestris L. Fruit Extract | 50-2000 | mg |
| with 40% Saponins | | |
| Turkey Tail | 2-3000 | mg |
| Undenatured type II collagen | 1-100 | mg |
| (UC-II) | | |
| Valerian Extract with 0.8% | 10-1000 | mg |
| valerenic acids | | |
| Verisol ® collagen | 1-2000 | mg |
| Vinpocetine 99% | 1-100 | mg |
| Wheat Germ Extract | 1-100,000 | mg |
| Wild Lettuce Powder | 0.1-2000 | mg |
| Willow Bark Dry Extract | 5-500 | mg |
| Zeaxanthin | 0.5-20 | mg |

### 7. EMBODIMENTS

Embodiment 1: A method of removing toxins in a patient as a result of chronic exposure to the toxins, comprising:
(a) performing one or more therapeutic plasma exchange (TPE) sessions on the patient; and
(b) administering an oral composition to the patient.

Embodiment 2: The method of embodiment 1, wherein each TPE session is performed with 0.8 to 1.5 estimated plasma volume (EPV).

Embodiment 3: The method of embodiment 2, wherein each TPE session is performed with 5% to 10% albumin.

Embodiment 4: The method of embodiment 3, wherein each TPE session is performed with an anticoagulant.

Embodiment 5: The method of embodiment 4, wherein the anticoagulant is citrate or heparin.

Embodiment 6: The method of embodiment 1 further comprising administering a metal chelator to the patient during step (a).

Embodiment 7: The method of embodiment 6, wherein the metal chelator is dimercaptosuccinic acid (DMSA), sodium 2,3 dimercaptopropanel sulphonate, ethylenediaminetetraacetic acid (EDTA), sodium calcium edetate, D-penicillamine, or N-acetyl-DL-penicillamine.

Embodiment 8: The method of embodiment 7, wherein the metal chelator is DMSA, and wherein the DMSA is administered to the patient at a dose of between 50 mg and 2000 mg.

Embodiment 9: The method of embodiment 8, wherein DMSA is administered to the patient at a dose of 250 mg daily for 3 days a week.

Embodiment 10: The method of embodiment 1, wherein the oral composition is administered to the patient daily after the first TPE session.

Embodiment 11: The method of embodiment 1, wherein the patient has atherosclerosis.

Embodiment 12: The method of embodiment 11, wherein the method is capable of reducing the level or slowing the progression of atherosclerosis in the patient.

Embodiment 13: The method of embodiment 11 further comprising administering at least one cholesterol-reducing agent to the patient after completing the first TPE session.

Embodiment 14: The method of embodiment 13, wherein the at least one cholesterol-reducing agent has not been administered to the patient prior to commencing the first TPE session.

Embodiment 15: The method of embodiment 14, wherein the at least one cholesterol-reducing agent is beta-cyclodextrin, and wherein beta-cyclodextrin is administered to the patient rectally at a dose of between 8 g and 16 g once a day for 6 months after completing the first TPE session.

Embodiment 16: The method of embodiment 13, wherein the at least one cholesterol-reducing agent is administered to the patient after completing the first TPE session.

Embodiment 17: The method of embodiment 1, wherein the patient has declined cognitive function.

Embodiment 18: The method of embodiment 17, wherein the method is capable of improving cognitive function in the patient.

Embodiment 19: The method of embodiment 1, wherein the method is capable of improving immune function in the patient.

Embodiment 20: The method of embodiment 1, wherein the patient has early cancer development.

Embodiment 21: The method of any one of embodiments 18 to 20 further comprising administering low dose naltrexone to the patient.

Embodiment 22: The method of embodiment 21, wherein the naltrexone is administered to the patient at a dose of between 1 mg and 20 mg.

Embodiment 23: The method of embodiment 22, wherein the low dose naltrexone is taken orally by the patient after completing the first TPE session, wherein the dose of naltrexone is 1.5 mg once daily for 2 weeks, wherein the dose of naltrexone is thereafter increased to 3.0 mg once daily for weeks, and wherein the dose of naltrexone is thereafter increased to 4.5 mg once daily for up to 52 weeks.

Embodiment 24: The method of embodiment 18 further comprising administering one or more neuropeptides to the patient prior to step (a).

Embodiment 25: The method of embodiment 24, wherein the neuropeptide is adrenocorticotropin, a tetrapeptide having the amino acid sequence of AEDG (SEQ ID NO: 1), a heptapeptide having the amino acid sequence of TKPRPGP (SEQ ID NO: 2), a tripeptide having the amino acid sequence of EDR, a tripeptide having the amino acid sequence of KED, fibroblast growth loop peptide (FGL), brain-derived neurotrophic factor (BDNF), glial cell line-derived neurotrophic factor (GDNF), nerve growth factor (NGF), ciliary neurotrophic factor (CNTF), hypocretin-1, low molecular peptides with a molecular weight up to 5000 Da isolated from the brain tissues of calves, or low molecular peptides with a molecular weight up to 10000 Da isolated from the vascular (aorta) tissue of calves, or a combination thereof.

Embodiment 26: The method of embodiment 25, wherein the neuropeptide is adrenocorticotropin (4-10).

Embodiment 27: The method of embodiment 26, wherein the adrenocorticotropin (4-10) is administered to the patient via intranasal spray at a dose of between 1 mcg and 2000 mcg.

Embodiment 28: The method of embodiment 25, wherein the neuropeptide is a tetrapeptide having the amino acid sequence of AEDG (SEQ ID NO: 1).

Embodiment 29: The method of embodiment 28, wherein the tetrapeptide is administered to the patient via subcutaneous injection or oral spray at a dose of between 0.1 mg and 50 mg.

Embodiment 30: The method of any one of embodiments 12, 18, or 19, wherein step (a) further comprises administering exosomes, mesenchymal stem cells (MSCs), or a combination thereof to the patient after completing at least one TPE session but no more than two consecutive TPE sessions.

Embodiment 31: The method of embodiment 30, wherein the MSCs are derived from umbilical cord.

Embodiment 32: The method of embodiment 30, wherein the exosomes are derived from umbilical cord.

Embodiment 33: The method of embodiment 30, wherein the exosomes, mesenchymal stem cells (MSCs), or a combination thereof is administered to the patient after completing at least one TPE session but no more than two consecutive TPE sessions.

Embodiment 34: The method of embodiment 20 further comprising administering at least one anti-inflammatory compound, one anti-fungal compound, or a combination thereof to the patient.

Embodiment 35: The method of embodiment 34 further comprising administering thymosin al, met enkephalin, methylene blue, nystatin, diflucan, colloidal silver, mebendazole, or a combination thereof to the patient.

Embodiment 36: The method of embodiment 35, wherein thymosin al is injected subcutaneously to the patient at a dose of between 0.5 mg and 3 mg once a day for 1 to 52 weeks.

Embodiment 37: The method of embodiment 36, wherein thymosin al is injected subcutaneously to the patient at a dose of 1.6 mg once a day for 1 to 52 weeks.

Embodiment 38: The method of embodiment 35, wherein met enkephaline is injected subcutaneously to the patient at a dose of between 0.5 mg and 3 mg once a day for 1 to 52 weeks.

Embodiment 39: The method of embodiment 38, wherein met enkephaline is injected subcutaneously to the patient at a dose of 1.5 mg once a day for 1 to 52 weeks.

Embodiment 40: The method of embodiment 35, wherein methylene blue is taken orally by the patient at a dose of between 1 mg and 100 mg once a day for 1 to 52 weeks.

Embodiment 41: The method of embodiment 40, wherein methylene blue is taken orally by the patient at a dose of 50 mg once a day for 1 to 52 weeks.

Embodiment 42: The method of embodiment 35, wherein nystatin liquid is taken orally by the patient at a dose of between 10,000 units and 4,000,000 units four times a day for 1 to 52 weeks.

Embodiment 43: The method of embodiment 42, wherein nystatin liquid is taken orally by the patient at a dose of 500,000 units four times a day for 1 to 52 weeks.

Embodiment 44: The method of embodiment 35, wherein diflucan is taken orally by the patient at a dose of between 50 mg and 200 mg once a day for 1 to 52 weeks.

Embodiment 45: The method of embodiment 44, wherein diflucan is taken orally by the patient at a dose of 100 mg once a day for 1 to 52 weeks.

Embodiment 46: The method of embodiment 35, wherein colloidal silver 2000 ppm is taken orally by the patient at a dose of between 1 mL and 100 mL four times a day for 1 to 52 weeks.

Embodiment 47: The method of embodiment 46, wherein colloidal silver 2000 ppm is taken orally by the patient at a dose of 10 mL four times a day for 1 to 52 weeks.

Embodiment 48: The method of embodiment 35, wherein mebendazole is taken orally by the patient at a dose of between 50 mg and 200 mg once a day for 1 to 52 weeks.

Embodiment 49: The method of embodiment 48, wherein mebendazole is taken orally by the patient at a dose of 200 mg once a day for 1 to 52 weeks.

Embodiment 50: The method of embodiment 1, wherein step (a) further comprises administering immunoglobulin to the patient after each TPE session.

Embodiment 51: The method of embodiment 50, wherein the immunoglobulin is administered via IV or subcutaneous infusion at a dose of between 1 g and 20 g.

Embodiment 52: The method of embodiment 1, wherein between 3 and 8 TPE sessions are performed and each TPE session is performed between 1 and 6 weeks after the preceding TPE session.

Embodiment 53: The method of embodiment 1 further comprising removing plasma from the patient at least once every 1 to 20 weeks after the last TPE session.

Embodiment 54: The method of embodiment 53, wherein the plasma is removed from the patient once every 1, 2, 3, 4, 5, or up to every 20 weeks after each TPE session but plasma is not removed from the patient more than once a week for 3 weeks in a row.

Embodiment 55: The method of embodiment 1, wherein the level of one or more toxins is reduced in the patient.

Embodiment 56: The method of embodiment 55, wherein the one or more toxins is a heavy metal, herbicide, pesticide, phenol, phthalate, volatile organic compound, mycotoxin, per- and/or polyfluorinated substance (PFAS), microplastic, or a combination thereof.

Embodiment 57: The method of embodiment 1, wherein the method is capable of increasing the level of one or more biomarkers indicating an improvement in immune response in the patient.

Embodiment 58: The method of embodiment 57, wherein the one or more biomarkers indicating an improvement in immune response is CD3, CD4, CD19, CD56, or a combination thereof.

Embodiment 59: The method of embodiment 1, wherein the method is capable of reducing the level of one or more biomarkers indicating a suppression of inflammation in the patient.

Embodiment 60: The method of embodiment 59, wherein the one or more biomarkers indicating suppression of inflammation is uric acid, hs-C reactive protein, interleukin-8, tumor necrosis factor alpha, or a combination thereof.

Embodiment 61: The method of embodiment 1, wherein the method is capable of increasing the level of one or more biomarkers associated with longevity in the patient.

Embodiment 62: The method of embodiment 61, wherein the one or more biomarkers associated with longevity is Klotho protein, NAD intracellular, or a combination thereof.

Embodiment 63: The method of embodiment 1, wherein the method is capable of reducing the level of one or more biomarkers associated with oxidative stress in the patient.

Embodiment 64: The method of embodiment 63, wherein the one or more biomarkers associated with oxidative stress is 8-oxoguanine, myeloperoxidase, or a combination thereof.

Embodiment 65: The method of embodiment 1, wherein the method is capable of increasing the level of one or more biomarkers associated with cellular senescence in the patient.

Embodiment 66: The method of embodiment 65, wherein the one or more biomarkers associated with cellular senescence is SA-β-galactosidase.

Embodiment 67: The method of embodiment 1, wherein each TPE session of step (a) further comprises:
(i) administering an intravenous (IV) composition to the patient after performing TPE on the patient; and
(ii) administering a glutathione solution to the patient.

Embodiment 68: The method of embodiment 67, wherein between 300 mL and 350 mL of the IV composition is administered to the patient.

Embodiment 69: The method of embodiment 68, wherein the IV composition has an osmolality of between 230 mOsm to 300 mOsm.

Embodiment 70: The method of embodiment 67, wherein the glutathione solution is administered to the patient by infusion 2 to 3 minutes after completing step (i).

Embodiment 71: The method of embodiment 70, wherein the glutathione solution is administered to the patient at a dose of between 1 mg and 5000 mg.

Embodiment 72: The method of embodiment 71, wherein the glutathione solution is administered to the patient at a dose of 1000 mg.

Embodiment 73: The method of embodiment 67, wherein the IV composition comprises:
(a) at least one mineral;
(b) at least one vitamin; and
(c) at least one amino acid.

Embodiment 74: The method of embodiment 73, wherein the IV composition comprises the mineral magnesium; the vitamins ascorbic acid, dexpanthenol, hydroxocobalamin, niacin, pyridoxine, riboflavin, and thiamine; and the amino acids carnitine and taurine.

Embodiment 75: The method of embodiment 73, wherein the at least one mineral is one or more of calcium, magnesium, molybdenum, potassium, selenium, and/or zinc.

Embodiment 76: The method of embodiment 75, wherein the at least one mineral is calcium gluconate or calcium chloride.

Embodiment 77: The method of embodiment 76, wherein the at least one mineral is calcium gluconate administered to the patient at a dose of between 1 mg and 5000 mg.

Embodiment 78: The method of embodiment 77, wherein calcium gluconate is administered to the patient at a dose of 1000 mg.

Embodiment 79: The method of embodiment 75, wherein the at least one mineral is magnesium chloride or magnesium sulfate.

Embodiment 80: The method of embodiment 75, wherein the at least one mineral is magnesium chloride administered to the patient at a dose of between 1 mg and 5000 mg.

Embodiment 81: The method of embodiment 80, wherein magnesium chloride is administered to the patient at a dose of 2100 mg.

Embodiment 82: The method of embodiment 75, wherein the at least mineral is molybdenum administered to the patient at a dose of between 1 mcg and 500 mcg.

Embodiment 83: The method of embodiment 82, wherein molybdenum is administered to the patient at a dose of 250 mcg.

Embodiment 84: The method of embodiment 75, wherein the at least one mineral is potassium chloride administered to the patient at a dose of between 1 meq and 40 meq.

Embodiment 85: The method of embodiment 84, wherein potassium chloride is administered to the patient at a dose of 6 meq.

Embodiment 86: The method of embodiment 75, wherein the at least one mineral is selenium administered to the patient at a dose of between 1 mcg and 800 mcg.

Embodiment 87: The method of embodiment 86, wherein selenium is administered to the patient at a dose of 400 mcg.

Embodiment 88: The method of embodiment 75, wherein the at least one mineral is zinc sulfate or zinc chloride.

Embodiment 89: The method of embodiment 88, wherein the at least one mineral is zinc sulfate administered to the patient at a dose of between 1 mg and 100 mg.

Embodiment 90: The method of embodiment 89, wherein zinc sulfate is administered to the patient at a dose of 5 mg.

Embodiment 91: The method of embodiment 73, wherein the at least one vitamin is one or more of ascorbic acid, dexpanthenol, hydroxocobalamin, niacin, pyridoxine, riboflavin, and/or thiamine.

Embodiment 92: The method of embodiment 91, wherein the at least one vitamin is ascorbic acid administered to the patient at a dose of between 1 mg and 25000 mg.

Embodiment 93: The method of embodiment 92, wherein ascorbic acid is administered to the patient at a dose of 15000 mg.

Embodiment 94: The method of embodiment 91, wherein the at least one vitamin is dexpanthenol administered to the patient at a dose of between 1 mg and 2000 mg.

Embodiment 95: The method of embodiment 94, wherein dexpanthenol is administered to the patient at a dose of 1500 mg.

Embodiment 96: The method of embodiment 91, wherein the at least one vitamin is hydroxocobalamin or methylcobalamin.

Embodiment 97: The method of embodiment 96, wherein the at least one vitamin is hydroxocobalamin administered to the patient at a dose of between 1 mg and 1000 mg.

Embodiment 98: The method of embodiment 97, wherein hydroxocobalamin is administered to the patient at a dose of 6 mg.

Embodiment 99: The method of embodiment 91, wherein the at least one vitamin is niacin or niacinamide.

Embodiment 100: The method of embodiment 99, wherein the at least one vitamin is niacin administered to the patient at a dose of between 1 mg and 1000 mg.

Embodiment 101: The method of embodiment 100, wherein niacin is administered to the patient at a dose of 300 mg.

Embodiment 102: The method of embodiment 91, wherein the at least one vitamin is pyridoxine administered to the patient at a dose of between 1 mg and 1000 mg.

Embodiment 103: The method of embodiment 102, wherein pyridoxine is administered to the patient at a dose of 100 mg.

Embodiment 104: The method of embodiment 91, wherein the at least one vitamin is riboflavin administered to the patient at a dose of between 1 mg and 100 mg.

Embodiment 105: The method of embodiment 104, wherein riboflavin is administered to the patient at a dose of 6 mg.

Embodiment 106: The method of embodiment 91, wherein the at least one vitamin is thiamine administered to the patient at a dose of between 1 mg and 1000 mg.

Embodiment 107: The method of embodiment 106, wherein thiamine is administered to the patient at a dose of 300 mg.

Embodiment 108: The method of embodiment 91, wherein the at least one amino acid is carnitine and/or taurine.

Embodiment 109: The method of embodiment 108, wherein the at least one amino acid is L-carnitine administered to the patient at a dose of between 1 mg and 2000 mg.

Embodiment 110: The method of embodiment 109, wherein L-carnitine is administered to the patient at a dose of 1000 mg.

Embodiment 111: The method of embodiment 108, wherein the at least one amino acid is taurine administered to the patient at a dose of between 1 mg and 5000 mg.

Embodiment 112: The method of embodiment 111, wherein taurine is administered to the patient at a dose of 400 mg.

Embodiment 113: The method of embodiment 1, wherein the oral composition comprises:
(a) at least one vitamin;
(b) at least one mineral;
(c) at least one anti-inflammatory agent;
(d) at least one antioxidant;
(e) at least one amino acid;
(f) at least one saccharide; and
(g) at least one herb.

Embodiment 114: The method of embodiment 113, wherein the oral composition further comprises at least one protein.

Embodiment 115: The method of embodiment 113, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, Schisandra berry extract, grape seed extract, micro pyrroloquinoline quinone, alpha lipoic acid, boswellin, and dandelion root extract; the amino acids arginine, glutamine, proline, and tyrosine; the saccharides larch arabinogalactan, ribose, and beta glucan; and the herbs gingerol and bitter melon extract.

Embodiment 116: The method of embodiment 113 further comprising vinpocetine and Panax ginseng, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, schisandra berry extract, grape seed extract, micro pyrroloquinoline quinone, alpha lipoic acid, boswellin, dandelion root extract, and Ginkgo biloba; the amino acids arginine, glutamine, proline, tyrosine, phosphatidylserine, and theanine; the saccharides larch arabinogalactan, ribose, and beta glucan; and the herbs gingerol, bitter melon extract, Hericium erinaceus, Withania somnifera, and Bacopa monnieri.

Embodiment 117: The method of embodiment 113, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, schisandra berry extract, grape seed extract, micro pyrroloquinoline quinone, alpha lipoic acid, boswellin, and dandelion root extract; the amino acids arginine, glutamine, proline, and tyrosine; the saccharides larch arabinogalactan, ribose, and beta glucan; and the herbs gingerol, bitter melon extract, Berberis aristata, and Citrus bergamia.

Embodiment 118: The method of any one of embodiments 115 to 117, wherein the oral composition further comprises at least one protein.

Embodiment 119: The method of either embodiment 114 or embodiment 118, wherein the protein is rice protein.

Embodiment 120: The method of embodiment 113, wherein the oral composition further comprises at least one other component.

Embodiment 121: The method of embodiment 120, wherein the at least one other component is vinpocentine or Panax ginseng.

Embodiment 122: The method of embodiment 120, wherein the at least one other component is vinpocentine administered to the patient at a dose of between 1 mg and 100 mg.

Embodiment 123: The method of embodiment 120, wherein vinpocentine is administered to the patient at a dose of 30 mg.

Embodiment 124: The method of embodiment 120, wherein the at least one other component is Panax ginseng, and wherein the Panax ginseng is Panax ginseng 5% Powdered Extract administered to the patient at a dose of between 1 mg and 2000 mg.

Embodiment 125: The method of embodiment 124, wherein Panax ginseng 5% Powdered Extract is administered to the patient at a dose of 100 mg.

Embodiment 126: The method of embodiment 113, wherein the at least one vitamin is one or more of biotin, cobalamin, folate, pantothenic acid, pyridoxine, riboflavin, thiamine, vitamin A, vitamin B3, vitamin C, vitamin D3, and/or vitamin E.

Embodiment 127: The method of embodiment 126, wherein the at least one vitamin is biotin administered to the patient at a dose of between 15 mcg and 7500 mcg.

Embodiment 128: The method of embodiment 127, wherein biotin is administered to the patient at a dose of 50 mcg.

Embodiment 129: The method of embodiment 126, wherein the at least one vitamin is cobalamin, and wherein the cobalamin is methylcobalamin administered to the patient at a dose of between 10 mcg and 10000 mcg.

Embodiment 130: The method of embodiment 129, wherein methylcobalamin is administered to the patient at a dose of 500 mcg.

Embodiment 131: The method of embodiment 126, wherein the at least one vitamin is folate, and wherein the folate is L-5-methyltetrahydrofolate glucosamine salt administered to the patient at a dose of between 10 mcg and 7500 mcg.

Embodiment 132: The method of embodiment 131, wherein L-5-methyltetrahydrofolate glucosamine salt is administered to the patient at a dose of 1000 mcg.

Embodiment 133: The method of embodiment 132, wherein the at least one vitamin is pantothenic acid administered to the patient at a dose of between 1 mg and 1500 mg.

Embodiment 134: The method of embodiment 133, wherein pantothenic acid is administered to the patient at a dose of 100 mg.

Embodiment 135: The method of embodiment 126, wherein the at least one vitamin is pyridoxine administered to the patient at a dose of between 1 mg and 300 mg.

Embodiment 136: The method of embodiment 135, wherein pyridoxine is administered to the patient at a dose of 15 mg.

Embodiment 137: The method of embodiment 126, wherein the at least one vitamin is riboflavin administered to the patient at a dose of between 1 mg and 500 mg.

Embodiment 138: The method of embodiment 137, wherein riboflavin is administered to the patient at a dose of 15 mg.

Embodiment 139: The method of embodiment 126, wherein the at least one vitamin is thiamine administered to the patient at a dose of between 1 mg and 300 mg.

Embodiment 140: The method of embodiment 139, wherein thiamine is administered to the patient at a dose of 1 mg.

Embodiment 141: The method of embodiment 126, wherein the at least one vitamin is vitamin A, and wherein the vitamin A is vitamin A palmitate administered to the patient at a dose of between 1 mg and 2000 mg.

Embodiment 142: The method of embodiment 141, wherein vitamin A palmitate is administered to the patient at a dose of 1000 mg.

Embodiment 143: The method of embodiment 126, wherein the at least one vitamin is vitamin B3, and wherein the vitamin B3 is niacin and/or nicotinamide riboside chloride.

Embodiment 144: The method of embodiment 143, wherein the at least one vitamin is niacin administered to the patient at a dose of between 1 mg and 750 mg.

Embodiment 145: The method of embodiment 144, wherein niacin is administered to the patient at a dose of 15 mg.

Embodiment 146: The method of embodiment 143, wherein the at least vitamin is nicotinamide riboside chloride administered to the patient at a dose of between 1 mg and 1000 mg.

Embodiment 147: The method of embodiment 146, wherein nicotinamide riboside chloride is administered to the patient at a dose of 750 mg.

Embodiment 148: The method of embodiment 126, wherein the at least one vitamin is vitamin C, and wherein the vitamin C is ascorbic acid administered to the patient at a dose of between 1 mg and 3000 mg.

Embodiment 149: The method of embodiment 148, wherein ascorbic acid is administered to the patient at a dose of 500 mg.

Embodiment 150: The method of embodiment 126, wherein the at least one vitamin is vitamin D3, and wherein the vitamin D3 is cholecalciferol administered to the patient at a dose of between 10 IU and 50000 IU.

Embodiment 151: The method of embodiment 150, wherein cholecalciferol is administered to the patient at a dose of 2500 IU.

Embodiment 152: The method of embodiment 126, wherein the at least one vitamin is vitamin E, and wherein the vitamin E is mixed tocopherols administered to the patient at a dose of between 10 IU and 500 IU.

Embodiment 153: The method of embodiment 152, wherein the mixed tocopherols are administered to the patient at a dose of 50 IU.

Embodiment 154: The method of embodiment 113, wherein the at least one mineral is one or more of calcium, chromium, copper, iodine, magnesium, manganese, molybdenum, and/or zinc.

Embodiment 155: The method of embodiment 154, wherein the at least one mineral is calcium, and wherein the calcium is calcium d-glucarate administered to the patient at a dose of between 25 mg and 1000 mg.

Embodiment 156: The method of embodiment 155, wherein calcium d-glucarate is administered to the patient at a dose of 500 mg.

Embodiment 157: The method of embodiment 154, wherein the at least one mineral is chromium, and wherein the chromium is chromium polynicotinate administered to the patient at a dose of between 10 mcg and 1000 mcg.

Embodiment 158: The method of embodiment 157, wherein chromium polynicotinate is administered to the patient at a dose of 25 mcg.

Embodiment 159: The method of embodiment 154, wherein the at least one mineral is copper, and wherein the copper is copper gluconate administered to the patient at a dose of between 0.1 mg and 10 mg.

Embodiment 160: The method of embodiment 159, wherein copper gluconate is administered to the patient at a dose of 1 mg.

Embodiment 161: The method of embodiment 154, wherein the at least one mineral is iodine, and wherein the iodine is potassium iodide administered to the patient at a dose of between 10 mcg and 500 mcg.

Embodiment 162: The method of embodiment 161, wherein potassium iodide administered to the patient at a dose of 25 mcg.

Embodiment 163: The method of embodiment 154, wherein the at least one mineral is magnesium, and wherein the magnesium is magnesium bisglycinate chelate buffered and/or magnesium glycinate.

Embodiment 164: The method of embodiment 163, wherein the at least one mineral is magnesium bisglycinate chelate buffered administered to the patient at a dose of between 2 mg and 2000 mg.

Embodiment 165: The method of embodiment 164, wherein magnesium bisglycinate chelate buffered is administered to the patient at a dose of 100 mg.

Embodiment 166: The method of embodiment 163, wherein the at least one mineral is magnesium glycinate administered to the patient at a dose of between 0.5 mg and 20 mg.

Embodiment 167: The method of embodiment 166, wherein magnesium glycinate is administered to the patient at a dose of 5 mg.

Embodiment 168: The method of embodiment 154, wherein the at least one mineral is molybdenum, and wherein the molybdenum is molybdenum glycinate chelate administered to the patient at a dose of between 1 mcg and 500 mcg.

Embodiment 169: The method of embodiment 168, wherein molybdenum glycinate chelate is administered to the patient at a dose of 50 mcg.

Embodiment 170: The method of embodiment 154, wherein the at least one mineral is zinc, and wherein the zinc is zinc bisglycinate chelate administered to the patient at a dose of between 1 mg and 50 mg.

Embodiment 171: The method of embodiment 170, wherein zinc bisglycinate chelate is administered to the patient at a dose of 5 mg.

Embodiment 172: The method of embodiment 113, wherein the at least one anti-inflammatory agent is one or more of baicalin, bromelain, Noni fruit extract, omega-3, oxindole alkaloid, pterostilbene, resveratrol, and/or superoxide dismutase.

Embodiment 173: The method of embodiment 172, wherein the at least one anti-inflammatory agent is baicalin, and wherein the baicalin is skullcap root extract containing 30% baicalin administered to the patient at a dose of between 25 mg to 1000 mg.

Embodiment 174: The method of embodiment 173, wherein skullcap root extract containing 30% baicalin is administered to the patient at a dose of 250 mg.

Embodiment 175: The method of embodiment 172, wherein the at least one anti-inflammatory agent is bromelain, and wherein the bromelain is bromelain 24000 gelatin dissolving units (GDU) administered to the patient at a dose of between 1 and 1000 mg.

Embodiment 176: The method of embodiment 175, wherein bromelain 24000 gelatin dissolving units (GDU) is administered to the patient at a dose of 50 mg.

Embodiment 177: The method of embodiment 172, wherein the at least one anti-inflammatory agent is Noni fruit extract, and wherein the Noni fruit extract is Noni fruit extract 4:1 administered to the patient at a dose of between 1 and 1000 mg.

Embodiment 178: The method of embodiment 177, wherein Noni fruit extract 4:1 is administered to the patient at a dose of 50 mg.

Embodiment 179: The method of embodiment 172, wherein the at least one anti-inflammatory agent is omega-3, and wherein the omega-3 is omega-3 comprising eicosapentaenoic acid (EPA) and docosahexaenoic acid (DHA).

Embodiment 180: The method of embodiment 179, wherein the omega-3 comprising eicosapentaenoic acid (EPA) and docosahexaenoic acid (DHA) is 2:1 EPA:DHA administered to the patient at a dose of between 25 mg and 3000 mg.

Embodiment 181: The method of embodiment 180, wherein the omega-3 comprising eicosapentaenoic acid (EPA) and docosahexaenoic acid (DHA) is 2:1 EPA:DHA administered to the patient at a dose of 200 mg.

Embodiment 182: The method of embodiment 172, wherein the at least one anti-inflammatory agent is oxindole alkaloids, and wherein the oxindole alkaloids is Unicaria tomentosa (Cat's claw) bark extract with 3% oxindole alkaloids administered to the patient at a dose of between 50 mg and 1200 mg.

Embodiment 183: The method of embodiment 182, wherein Unicaria tomentosa (Cat's claw) bark extract with 3% oxindole alkaloids is administered to the patient at a dose of 250 mg.

Embodiment 184: The method of embodiment 172, wherein the at least one anti-inflammatory agent is pterostilbene administered to the patient at a dose of between 10 mg and 250 mg.

Embodiment 185: The method of embodiment 184, wherein pterostilbene is administered to the patient at a dose of 50 mg.

Embodiment 186: The method of embodiment 172, wherein the at least one anti-inflammatory agent is resveratrol, and wherein the resveratrol is resveratrol root extract administered to the patient at a dose of between 5 mg and 2000 mg.

Embodiment 187: The method of embodiment 186, wherein resveratrol root extract is administered to the patient at a dose of 100 mg.

Embodiment 188: The method of embodiment 172, wherein the at least one anti-inflammatory agent is superoxide dismutase, and wherein the superoxide dismutase is 14000 IU/g superoxide dismutase (SOD) administered to the patient at a dose of between 1 mg and 2000 mg.

Embodiment 189: The method of embodiment 188, wherein 14000 IU/g superoxide dismutase (SOD) is administered to the patient at a dose of 75 mg.

Embodiment 190: The method of embodiment 113, wherein the at least one antioxidant is one or more of alpha lipoic acid, astaxanthin, Boswellia serrata extract, coenzyme Q10, Curcuma longa extract, cysteine, dandelion root extract, epigallocatechin gallate, Ginkgo biloba, glucoraphanin, glutathione, grape seed extract, hops extract, micro pyrroloquinoline quinone, polyphenols, punicalagin, quercetin, rosemary extract, schisandra berry extract, shilajit extract, and/or silymarin.

Embodiment 191: The method of embodiment 190, wherein the at least one antioxidant is alpha lipoic acid administered to the patient at a dose of between 5 mg and 1800 mg.

Embodiment 192: The method of embodiment 191, wherein alpha lipoic acid is administered to the patient at a dose of 100 mg.

Embodiment 193: The method of embodiment 190, wherein the at least one antioxidant is astaxanthin administered to the patient at a dose of between 1 mg and 20 mg.

Embodiment 194: The method of embodiment 193, wherein astaxanthin is administered to the patient at a dose of 5 mg.

Embodiment 195: The method of embodiment 190, wherein the at least one antioxidant is Boswellia serrata extract administered to the patient at a dose of between 5 mg and 7000 mg.

Embodiment 196: The method of embodiment 195, wherein Boswellia serrata extract is administered to the patient at a dose of 1000 mg.

Embodiment 197: The method of embodiment 190, wherein the at least one antioxidant is coenzyme Q10, and wherein the coenzyme Q10 is coenzyme Q10 blend from sunflower lecithin administered to the patient at a dose of between 10 mg and 1000 mg.

Embodiment 198: The method of embodiment 197, wherein coenzyme Q10 blend from sunflower lecithin is administered to the patient at a dose of 50 mg.

Embodiment 199: The method of embodiment 190, wherein the at least one antioxidant is Curcuma longa Extract administered to the patient at a dose of between 50 mg and 5000 mg.

Embodiment 200: The method of embodiment 199, wherein Curcuma longa Extract is administered to the patient at a dose of 250 mg.

Embodiment 201: The method of embodiment 190, wherein the at least one antioxidant is cysteine, and wherein the cysteine is N-Acetyl-L-Cysteine (NAC) administered to the patient at a dose of between 5 mg and 2000 mg.

Embodiment 202: The method of embodiment 201, wherein NAC is administered to the patient at a dose of 100 mg.

Embodiment 203: The method of embodiment 190, wherein the at least one antioxidant is dandelion root extract administered to the patient at a dose of between 20 mg and 1000 mg.

Embodiment 204: The method of embodiment 203, wherein dandelion root extract is administered to the patient at a dose of 250 mg.

Embodiment 205: The method of embodiment 190, wherein the at least one antioxidant is epigallocatechin gallate (EGCG), and wherein the EGCG is Camellia sinensis leaf extract with 50% EGCG administered to the patient at a dose of between 10 mg and 1000 mg.

Embodiment 206: The method of embodiment 205, wherein Camellia sinensis leaf extract with 50% EGCG is administered to the patient at a dose of 100 mg.

Embodiment 207: The method of embodiment 190, wherein the at least one antioxidant is Ginkgo biloba, and wherein the Ginkgo biloba is Ginkgo biloba leaf extract with 24% Ginkoflavonglycosides/6% Terpene Lactones administered to the patient at a dose of between 30 mg and 350 mg.

Embodiment 208: The method of embodiment 207, wherein Ginkgo biloba leaf extract with 24% Ginkoflavonglycosides/6% Terpene Lactones is administered to the patient at a dose of 120 mg.

Embodiment 209: The method of embodiment 190, wherein the at least one antioxidant is glucoraphanin, and wherein the glucoraphanin is broccoli seed extract with 13% glucoraphanin administered to the patient at a dose of between 25 mg and 300 mg.

Embodiment 210: The method of embodiment 209, wherein broccoli seed extract with 13% glucoraphanin is administered to the patient at a dose of 75 mg.

Embodiment 211: The method of embodiment 190, wherein the at least one antioxidant is glutathione, and wherein the glutathione is reduced L-glutathione administered to the patient at a dose of between 50 mg and 1000 mg.

Embodiment 212: The method of embodiment 211, wherein reduced L-glutathione administered to the patient at a dose of 100 mg.

Embodiment 213: The method of embodiment 190, wherein the at least one antioxidant is grape seed extract administered to the patient at a dose of between 5 mg and 1000 mg.

Embodiment 214: The method of embodiment 213, wherein grape seed extract is administered to the patient at a dose of 100 mg.

Embodiment 215: The method of embodiment 190, wherein the at least one antioxidant is hops extract administered to the patient at a dose of between 25 mg and 1000 mg.

Embodiment 216: The method of embodiment 215, wherein hops extract is administered to the patient at a dose of 125 mg.

Embodiment 217: The method of embodiment 190, wherein the at least one antioxidant is micro pyrroloquinoline quinone administered to the patient at a dose of between 1 mg and 800 mg.

Embodiment 218: The method of embodiment 217, wherein micro pyrroloquinoline quinone is administered to the patient at a dose of 10 mg.

Embodiment 219: The method of embodiment 190, wherein the at least one antioxidant is polyphenols, and wherein the polyphenols are artichoke flower extract containing 3% polyphenols administered to the patient at a dose of between 20 mg and 1000 mg.

Embodiment 220: The method of embodiment 219, wherein artichoke flower extract containing 3% polyphenols is administered to the patient at a dose of 100 mg.

Embodiment 221: The method of embodiment 190, wherein the at least one antioxidant is punicalagins, and wherein the punicalagins is Punica granatum fruit extract containing 30% punicalagins administered to the patient at a dose of between 10 mg and 1500 mg.

Embodiment 222: The method of embodiment 193, wherein Punica granatum fruit extract containing 30% punicalagins is administered to the patient at a dose of 250 mg.

Embodiment 223: The method of embodiment 190, wherein the at least one antioxidant is quercetin administered to the patient at a dose of between 10 mg and 2000 mg.

Embodiment 224: The method of embodiment 195, wherein quercetin is administered to the patient at a dose of 500 mg.

Embodiment 225: The method of embodiment 190, wherein the at least one antioxidant is rosemary extract administered to the patient at a dose of between 5 mg and 750 mg.

Embodiment 226: The method of embodiment 197, wherein rosemary extract is administered to the patient at a dose of 100 mg.

Embodiment 227: The method of embodiment 190, wherein the at least one antioxidant is Schisandra berry extract administered to the patient at a dose of between 20 mg and 3000 mg.

Embodiment 228: The method of embodiment 199, wherein Schisandra berry extract is administered to the patient at a dose of 100 mg.

Embodiment 229: The method of embodiment 190, wherein the at least one antioxidant is shilajit extract administered to the patient at a dose of between 20 mg and 1500 mg.

Embodiment 230: The method of embodiment 201, wherein shilajit extract is administered to the patient at a dose of 100 mg.

Embodiment 231: The method of embodiment 190, wherein the at least one antioxidant is silymarin, and wherein the silymarin is Silybum marianumshila extract containing 80% silymarin administered to the patient at a dose of between 30 mg and 2400 mg.

Embodiment 232: The method of embodiment 203, wherein Silybum marianumshila extract containing 80% silymarin is administered to the patient at a dose of 100 mg.

Embodiment 233: The method of embodiment 113, wherein the at least one amino acid is one or more of alanine, arginine, glutamine, glycine, lysine, methionine, ornithine, phosphatidylserine, proline, tyrosine, taurine, theanine, and/or trimethyl glycine.

Embodiment 234: The method of embodiment 233, wherein the at least one amino acid is alanine, and wherein the alanine is L-alanine administered to the patient at a dose of between 50 mg and 1000 mg.

Embodiment 235: The method of embodiment 234, wherein L-alanine is administered to the patient at a dose of 175 mg.

Embodiment 236: The method of embodiment 233, wherein the at least one amino acid is arginine, and wherein the arginine is L-arginine hydrochloride administered to the patient at a dose of between 5 mg and 3000 mg.

Embodiment 237: The method of embodiment 236, wherein L-arginine hydrochloride is administered to the patient at a dose of 100 mg.

Embodiment 238: The method of embodiment 233, wherein the at least one amino acid is L-glutamine administered to the patient at a dose of between 1 mg and 3000 mg.

Embodiment 239: The method of embodiment 238, wherein L-glutamine is administered to the patient at a dose of 1250 mg.

Embodiment 240: The method of embodiment 233, wherein the at least one amino acid is L-glycine administered to the patient at a dose of between 1 mg and 6000 mg.

Embodiment 241: The method of embodiment 240, wherein L-glycine is administered to the patient at a dose of 300 mg.

Embodiment 242: The method of embodiment 233, wherein the at least one amino acid is lysine, and wherein the lysine is L-lysine hydrochloride administered to the patient at a dose of between 5 mg and 2000 mg.

Embodiment 243: The method of embodiment 242, wherein L-lysine hydrochloride is administered to the patient at a dose of 500 mg.

Embodiment 244: The method of embodiment 233, wherein the at least one amino acid is methionine, and wherein the methionine is L-methionine administered to the patient at a dose of between 5 mg and 1500 mg.

Embodiment 245: The method of embodiment 244, wherein L-methionine is administered to the patient at a dose of 70 mg.

Embodiment 246: The method of embodiment 233, wherein the at least one amino acid is ornithine, and wherein the ornithine is L-ornithine hydrochloride administered to the patient at a dose of between 1 mg and 1000 mg.

Embodiment 247: The method of embodiment 246, wherein L-ornithine hydrochloride is administered to the patient at a dose of 50 mg.

Embodiment 248: The method of embodiment 233, wherein the at least one amino acid is phosphatidylserine administered to the patient at a dose of between 10 mg and 2000 mg.

Embodiment 249: The method of embodiment 248, wherein phosphatidylserine is administered to the patient at a dose of 100 mg.

Embodiment 250: The method of embodiment 233, wherein the at least one amino acid is proline, and wherein the proline is L-proline administered to the patient at a dose of between 5 mg and 1000 mg.

Embodiment 251: The method of embodiment 250, wherein L-proline is administered to the patient at a dose of 250 mg.

Embodiment 252: The method of embodiment 233, wherein the at least one amino acid is tyrosine, and wherein the tyrosine is L-tyrosine administered to the patient at a dose of between 5 mg and 1000 mg.

Embodiment 253: The method of embodiment 252, wherein L-tyrosine is administered to the patient at a dose of 50 mg.

Embodiment 254: The method of embodiment 233, wherein the at least one amino acid is taurine administered to the patient at a dose of between 10 mg and 2000 mg.

Embodiment 255: The method of embodiment 254, wherein taurine is administered to the patient at a dose of 100 mg.

Embodiment 256: The method of embodiment 233, wherein the at least one amino acid is theanine, and wherein the theanine is L-theanine administered to the patient at a dose of between 5 mg and 2500 mg.

Embodiment 257: The method of embodiment 256, wherein L-theanine is administered to the patient at a dose of 100 mg.

Embodiment 258: The method of embodiment 233, wherein the at least one amino acid is trimethyl glycine administered to the patient at a dose of between 10 mg and 1500 mg.

Embodiment 259: The method of embodiment 258, wherein trimethyl glycine is administered to the patient at a dose of 1000 mg.

Embodiment 260: The method of embodiment 113, wherein the at least one saccharide is one or more of larch arabinogalactan, beta glucan, and/or ribose.

Embodiment 261: The method of embodiment 260, wherein the at least one saccharide is larch arabinogalactan administered to the patient at a dose of between 50 mg and 2000 mg.

Embodiment 262: The method of embodiment 261, wherein larch arabinogalactan is administered to the patient at a dose of 500 mg.

Embodiment 263: The method of embodiment 260, wherein the at least one saccharide is beta glucan, and wherein the beta glucan is 85% 1,3 beta glucan administered to the patient at a dose of between 7.5 mg and 1000 mg.

Embodiment 264: The method of embodiment 263, wherein 85% 1,3 beta glucan is administered to the patient at a dose of 500 mg.

Embodiment 265: The method of embodiment 260, wherein the at least one saccharide is ribose, and wherein the ribose is D-ribose administered to the patient at a dose of between 50 mg and 5000 mg.

Embodiment 266: The method of embodiment 265, wherein D-ribose is administered to the patient at a dose of 1000 mg.

Embodiment 267: The method of embodiment 113, wherein the at least one herb is Bacopa monnieri, Berberis aristata, bitter melon extract, Citrus bergamia, gingerol, Hericium erinaceus, and/or Withania somnifera.

Embodiment 268: The method of embodiment 267, wherein the at least one herb is Bacopa monnieri extract administered to the patient at a dose of between 50 mg and 500 mg.

Embodiment 269: The method of embodiment 268, wherein Bacopa monnieri extract is administered to the patient at a dose of 100 mg.

Embodiment 270: The method of embodiment 267, wherein the at least one herb is Berberis aristata root extract, and wherein the Berberis aristata root extract is Berberis aristata root extract with 97% Berberine HCl administered to the patient at a dose of between 20 mg and 2000 mg.

Embodiment 271: The method of embodiment 270, wherein Berberis aristata root extract with 97% Berberine HCl is administered to the patient at a dose of 500 mg.

Embodiment 272: The method of embodiment 267, wherein the at least one herb is bitter melon extract administered to the patient at a dose of between 50 mg and 1000 mg.

Embodiment 273: The method of embodiment 272, wherein bitter melon extract is administered to the patient at a dose of 100 mg.

Embodiment 274: The method of embodiment 267, wherein the at least one herb is Citrus bergamia extract, and wherein the Citrus bergamia extract is Citrus bergamia with 40% Polyphenol Fraction administered to the patient at a dose of between 1 mg and 2600 mg.

Embodiment 275: The method of embodiment 274, wherein Citrus bergamia with 40% Polyphenol Fraction is administered to the patient at a dose of 500 mg.

Embodiment 276: The method of embodiment 267, wherein the at least one herb is gingerols, and wherein the gingerols is Ginger rhizome extract containing 5% gingerols administered to the patient at a dose of between 10 mg and 1000 mg.

Embodiment 277: The method of embodiment 276, wherein Ginger rhizome extract containing 5% gingerols is administered to the patient at a dose of 200 mg.

Embodiment 278: The method of embodiment 267, wherein the at least one herb is Hericium erinaceus extract administered to the patient at a dose of between 1 mg and 3000 mg.

Embodiment 279: The method of embodiment 278, wherein Hericium erinaceus extract is administered to the patient at a dose of 100 mg.

Embodiment 280: The method of embodiment 267, wherein the at least one herb is Withania somnifera root extract administered to the patient at a dose of between 50 mg and 2000 mg.

Embodiment 281: The method of embodiment 280, wherein Withania somnifera root extract is administered to the patient at a dose of 100 mg.

Embodiment 282: An IV composition for detoxification in a patient, comprising one or more of:
(a) at least one mineral;
(b) at least one vitamin; and
(c) at least one amino acid.

Embodiment 283: The IV composition of embodiment 282, wherein the total volume of the IV composition is between 300 mL and 350 mL.

Embodiment 284: The IV composition of embodiment 282, wherein the osmolality of the IV composition is between 230 mOsm to 300 mOsm.

Embodiment 285: The IV composition of embodiment 282, wherein the IV composition comprises the mineral magnesium; the vitamins ascorbic acid, dexpanthenol, hydroxocobalamin, niacin, pyridoxine, riboflavin, and thiamine; and the amino acids carnitine and taurine.

Embodiment 286: The IV composition of embodiment 282, wherein the at least one mineral is one or more of calcium, magnesium, molybdenum, potassium, selenium, and/or zinc.

Embodiment 287: The IV composition of embodiment 286, wherein the at least one mineral is calcium gluconate or calcium chloride.

Embodiment 288: The IV composition of embodiment 287, wherein the at least one mineral is calcium gluconate at a dose of between 1 mg and 5000 mg.

Embodiment 289: The IV composition of embodiment 288, wherein the dose of calcium gluconate is 1000 mg.

Embodiment 290: The IV composition of embodiment 286, wherein the at least one mineral is magnesium chloride or magnesium sulfate.

Embodiment 291: The IV composition of embodiment 290, wherein the at least one mineral is magnesium chloride at a dose of between 1 mg and 5000 mg.

Embodiment 292: The IV composition of embodiment 291, wherein the dose of magnesium chloride is 2100 mg.

Embodiment 293: The IV composition of embodiment 286, wherein the at least mineral is molybdenum at a dose of between 1 mcg and 500 mcg.

Embodiment 294: The IV composition of embodiment 293, wherein the dose of molybdenum is 250 mcg.

Embodiment 295: The IV composition of embodiment 286, wherein the at least one mineral is potassium chloride at a dose of between 1 meq and 40 meq.

Embodiment 296: The IV composition of embodiment 295, wherein the dose of potassium chloride is 6 meq.

Embodiment 297: The IV composition of embodiment 286, wherein the at least one mineral is selenium at a dose of between 1 mcg and 800 mcg.

Embodiment 298: The IV composition of embodiment 297, wherein the dose of selenium is 400 mcg.

Embodiment 299: The IV composition of embodiment 286, wherein the at least one mineral is zinc sulfate or zinc chloride.

Embodiment 300: The IV composition of embodiment 299, wherein the at least one mineral is zinc sulfate at a dose of between 1 mg and 100 mg.

Embodiment 301: The IV composition of embodiment 300, wherein the dose of zinc sulfate is 5 mg.

Embodiment 302: The IV composition of embodiment 282, wherein the at least one vitamin is one or more of ascorbic acid, dexpanthenol, hydroxocobalamin, niacin, pyridoxine, riboflavin, and/or thiamine.

Embodiment 303: The IV composition of embodiment 302, wherein the at least one vitamin is ascorbic acid at a dose of between 1 mg and 25000 mg.

Embodiment 304: The IV composition of embodiment 303, wherein the dose of ascorbic acid is 15000 mg.

Embodiment 305: The IV composition of embodiment 302, wherein the at least one vitamin is dexpanthenol at a dose of between 1 mg and 2000 mg.

Embodiment 306: The IV composition of embodiment 305, wherein the dose of dexpanthenol is 1500 mg.

Embodiment 307: The IV composition of embodiment 302, wherein the at least one vitamin is hydroxocobalamin or methylcobalamin.

Embodiment 308: The IV composition of embodiment 307, wherein the at least one vitamin is hydroxocobalamin at a dose of between 1 mg and 1000 mg.

Embodiment 309: The IV composition of embodiment 308, wherein the dose of hydroxocobalamin is 6 mg.

Embodiment 310: The IV composition of embodiment 302, wherein the at least one vitamin is niacin or niacinamide.

Embodiment 311: The IV composition of embodiment 310, wherein the at least one vitamin is niacin at a dose of between 1 mg and 1000 mg.

Embodiment 312: The IV composition of embodiment 311, wherein the dose of niacin is 300 mg.

Embodiment 313: The IV composition of embodiment 302, wherein the at least one vitamin is pyridoxine at a dose of between 1 mg and 1000 mg.

Embodiment 314: The IV composition of embodiment 313, wherein the dose of pyridoxine is 100 mg.

Embodiment 315: The IV composition of embodiment 302, wherein the at least one vitamin is riboflavin at a dose of between 1 mg and 100 mg.

Embodiment 316: The IV composition of embodiment 315, wherein the dose of riboflavin is 6 mg.

Embodiment 317: The IV composition of embodiment 302, wherein the at least one vitamin is thiamine at a dose of between 1 mg and 1000 mg.

Embodiment 318: The IV composition of embodiment 317, wherein the dose of thiamine is 300 mg.

Embodiment 319: The IV composition of embodiment 282, wherein the at least one amino acid is carnitine and/or taurine.

Embodiment 320: The IV composition of embodiment 319, wherein the at least one amino acid is L-carnitine at a dose of between 1 mg and 2000 mg.

Embodiment 321: The IV composition of embodiment 320, wherein the dose of L-carnitine is 1000 mg.

Embodiment 322: The IV composition of embodiment 319, wherein the at least one amino acid is taurine at a dose of between 1 mg and 5000 mg.

Embodiment 323: The IV composition of embodiment 322, wherein the dose of taurine is 400 mg.

Embodiment 324: An oral composition for detoxification in a patient, comprising one or more of:
(a) at least one vitamin;
(b) at least one mineral;
(c) at least one anti-inflammatory agent;
(d) at least one antioxidant;
(e) at least one amino acid;
(f) at least one saccharide; and
(g) at least one herb.

Embodiment 325: The oral composition of embodiment 324 further comprising at least one protein.

Embodiment 326: The oral composition of embodiment 324, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, schisandra berry extract, grape seed extract, micro pyrroloquinoline quinone, alpha lipoic acid, boswellin, and dandelion root extract; the amino acids arginine, glutamine, proline, and tyrosine; the saccharides larch arabinogalactan, ribose, and beta glucan; and the herbs gingerol or bitter melon extract.

Embodiment 327: The oral composition of embodiment 324 further comprising vinpocetine and Panax ginseng, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, schisandra berry extract, grape seed extract, micro pyrroloquinoline quinone, alpha lipoic acid, boswellin, dandelion root extract, and Ginkgo biloba; the amino acids arginine, glutamine, proline, tyrosine, phosphatidylserine, and L-theanine; the saccharides larch arabinogalactan, ribose, and beta glucan; and the herbs gingerol, bitter melon extract, Hericium erinaceus, Withania somnifera, and Bacopa monnieri.

Embodiment 328: The oral composition of embodiment 324, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, Schisandra berry extract, grape seed extract, micro pyrroloquinoline quinone, alpha lipoic acid, boswellin, and dandelion root extract; the amino acids arginine, glutamine, proline, and tyrosine; the saccharides larch arabinogalactan, ribose, and beta glucan; and the herbs gingerol, bitter melon extract, Berberis aristata, and Citrus bergamia.

Embodiment 329: The oral composition of any one of embodiments 326 to 328, wherein the oral composition further comprises at least one protein.

Embodiment 330: The oral composition of either embodiment 325 or embodiment 329, wherein the protein is rice protein.

Embodiment 331: The oral composition of embodiment 324, wherein the oral composition further comprises at least one other component.

Embodiment 332: The oral composition of embodiment 331, wherein the at least one other component is vinpocentine or Panax ginseng.

Embodiment 333: The oral composition of embodiment 324, wherein the at least one other component is vinpocentine at a dose of between 1 mg and 100 mg.

Embodiment 334: The oral composition of embodiment 333, wherein the dose of vinpocentine is 30 mg.

Embodiment 335: The oral composition of embodiment 324, wherein the at least one other component is Panax ginseng, and wherein the Panax ginseng is Panax ginseng 5% Powdered Extract at a dose of between 1 mg and 2000 mg.

Embodiment 336: The oral composition of embodiment 335, wherein the dose of Panax ginseng 5% Powdered Extract is 100 mg.

Embodiment 337: The oral composition of embodiment 324, wherein the at least one vitamin is one or more of biotin, cobalamin, folate, pantothenic acid, pyridoxine, riboflavin, thiamine, vitamin A, vitamin B3, vitamin C, vitamin D3, and/or vitamin E.

Embodiment 338: The oral composition of embodiment 337, wherein the at least one vitamin is biotin at a dose of between 15 mcg and 7500 mcg.

Embodiment 339: The oral composition of embodiment 338, wherein the dose of biotin is 50 mcg.

Embodiment 340: The oral composition of embodiment 337, wherein the at least one vitamin is cobalamin, and wherein the cobalamin is methylcobalamin at a dose of between 10 mcg and 10000 mcg.

Embodiment 341: The oral composition of embodiment 340, wherein the dose of methylcobalamin is 500 mcg.

Embodiment 342: The oral composition of embodiment 337, wherein the at least one vitamin is folate, and wherein the folate is L-5-methyltetrahydrofolate glucosamine salt at a dose of between 10 mcg and 7500 mcg.

Embodiment 343: The oral composition of embodiment 342, wherein the dose of L-5-methyltetrahydrofolate glucosamine salt is 1000 mcg.

Embodiment 344: The oral composition of embodiment 337, wherein the at least one vitamin is pantothenic acid at a dose of between 1 mg and 1500 mg.

Embodiment 345: The oral composition of embodiment 344, wherein the dose of pantothenic acid is 100 mg.

Embodiment 346: The oral composition of embodiment 337, wherein the at least one vitamin is pyridoxine at a dose of between 1 mg and 300 mg.

Embodiment 347: The oral composition of embodiment 346, wherein the dose of pyridoxine is 15 mg.

Embodiment 348: The oral composition of embodiment 337, wherein the at least one vitamin is riboflavin at a dose of between 1 mg and 500 mg.

Embodiment 349: The oral composition of embodiment 348, wherein the dose of riboflavin is 15 mg.

Embodiment 350: The oral composition of embodiment 337, wherein the at least one vitamin is thiamine at a dose of between 1 mg and 300 mg.

Embodiment 351: The oral composition of embodiment 350, wherein the dose of thiamine is 1 mg.

Embodiment 352: The oral composition of embodiment 337, wherein the at least one vitamin is vitamin A, and wherein the vitamin A is vitamin A palmitate at a dose of between 1 mg and 2000 mg.

Embodiment 353: The oral composition of embodiment 352, wherein the dose of vitamin A palmitate is 1000 mg.

Embodiment 354: The oral composition of embodiment 337, wherein the at least one vitamin is vitamin B3, and wherein the vitamin B3 is niacin and/or nicotinamide riboside chloride.

Embodiment 355: The oral composition of embodiment 354, wherein the at least one vitamin is niacin at a dose of between 1 mg and 750 mg.

Embodiment 356: The oral composition of embodiment 355, wherein the dose of niacin is 15 mg.

Embodiment 357: The oral composition of embodiment 354, wherein the at least vitamin is nicotinamide riboside chloride at a dose of between 1 mg and 1000 mg.

Embodiment 358: The oral composition of embodiment 357, wherein the dose of nicotinamide riboside chloride is 750 mg.

Embodiment 359: The oral composition of embodiment 337, wherein the at least one vitamin is vitamin C, and wherein the vitamin C is ascorbic acid at a dose of between 1 mg and 3000 mg.

Embodiment 360: The oral composition of embodiment 359, wherein the dose of ascorbic acid is 500 mg.

Embodiment 361: The oral composition of embodiment 337, wherein the at least one vitamin is vitamin D3, and wherein the vitamin D3 is cholecalciferol at a dose of between 10 IU and 50000 IU.

Embodiment 362: The oral composition of embodiment 361, wherein the dose of cholecalciferol is 2500 IU.

Embodiment 363: The oral composition of embodiment 337, wherein the at least one vitamin is vitamin E, and wherein the vitamin E is mixed tocopherols at a dose of between 10 IU and 500 IU.

Embodiment 364: The oral composition of embodiment 363, wherein the dose of mixed tocopherols is 50 IU.

Embodiment 365: The oral composition of embodiment 324, wherein the at least one mineral is one or more of calcium, chromium, copper, iodine, magnesium, manganese, molybdenum, and/or zinc.

Embodiment 366: The oral composition of embodiment 365, wherein the at least one mineral is calcium, and wherein the calcium is calcium d-glucarate at a dose of between 25 mg and 1000 mg.

Embodiment 367: The oral composition of embodiment 366, wherein the dose of calcium d-glucarate is 500 mg.

Embodiment 368: The oral composition of embodiment 365, wherein the at least one mineral is chromium, and wherein the chromium is chromium polynicotinate at a dose of between 10 mcg and 1000 mcg.

Embodiment 369: The oral composition of embodiment 368, wherein the dose of chromium polynicotinate is 25 mcg.

Embodiment 370: The oral composition of embodiment 365, wherein the at least one mineral is copper, and wherein the copper is copper gluconate at a dose of between 0.1 mg and 10 mg.

Embodiment 371: The oral composition of embodiment 370, wherein the dose of copper gluconate is 1 mg.

Embodiment 372: The oral composition of embodiment 365, wherein the at least one mineral is iodine, and wherein the iodine is potassium iodide at a dose of between 10 mcg and 500 mcg.

Embodiment 373: The oral composition of embodiment 372, wherein the dose of potassium iodide is 25 mcg.

Embodiment 374: The oral composition of embodiment 365, wherein the at least one mineral is magnesium, and wherein the magnesium is magnesium bisglycinate chelate buffered and/or magnesium glycinate.

Embodiment 375: The oral composition of embodiment 374, wherein the at least one mineral is magnesium bisglycinate chelate buffered at a dose of between 2 mg and 2000 mg.

Embodiment 376: The oral composition of embodiment 375, wherein the dose of magnesium bisglycinate chelate buffered is 100 mg.

Embodiment 377: The oral composition of embodiment 374, wherein the at least one mineral is magnesium glycinate at a dose of between 0.5 mg and 20 mg.

Embodiment 378: The oral composition of embodiment 377, wherein the dose of magnesium glycinate is 5 mg.

Embodiment 379: The oral composition of embodiment 365, wherein the at least one mineral is molybdenum, and wherein the molybdenum is molybdenum glycinate chelate at a dose of between 1 mcg and 500 mcg.

Embodiment 380: The oral composition of embodiment 379, wherein the dose of molybdenum glycinate chelate is 50 mcg.

Embodiment 381: The oral composition of embodiment 365, wherein the at least one mineral is zinc, and wherein the zinc is zinc bisglycinate chelate at a dose of between 1 mg and 50 mg.

Embodiment 382: The oral composition of embodiment 381, wherein the dose of zinc bisglycinate chelate is 5 mg.

Embodiment 383: The oral composition of embodiment 324, wherein the at least one anti-inflammatory agent is one or more of baicalin, bromelain, Noni fruit extract, omega-3, oxindole alkaloid, pterostilbene, resveratrol, and/or superoxide dismutase.

Embodiment 384: The oral composition of embodiment 383, wherein the at least one anti-inflammatory agent is baicalin, and wherein the baicalin is skullcap root extract containing 30% baicalin at a dose of between 25 mg to 1000 mg.

Embodiment 385: The oral composition of embodiment 384, wherein the dose of skullcap root extract containing 30% baicalin is 250 mg.

Embodiment 386: The oral composition of embodiment 383, wherein the at least one anti-inflammatory agent is bromelain, and wherein the bromelain is bromelain 24000 gelatin dissolving units (GDU) at a dose of between 1 and 1000 mg.

Embodiment 387: The oral composition of embodiment 386, wherein the dose of bromelain 24000 gelatin dissolving units (GDU) is 50 mg.

Embodiment 388: The oral composition of embodiment 383, wherein the at least one anti-inflammatory agent is Noni fruit extract, and wherein the Noni fruit extract is Noni fruit extract 4:1 at a dose of between 1 and 1000 mg.

Embodiment 389: The oral composition of embodiment 388, wherein the dose of Noni fruit extract 4:1 is 50 mg.

Embodiment 390: The oral composition of embodiment 383, wherein the at least one anti-inflammatory agent is omega-3, and wherein the omega-3 is omega-3 comprising eicosapentaenoic acid (EPA) and docosahexaenoic acid (DHA).

Embodiment 391: The oral composition of embodiment 390, wherein the omega-3 comprising eicosapentaenoic acid (EPA) and docosahexaenoic acid (DHA) is 2:1 EPA:DHA at a dose of between 25 mg and 3000 mg.

Embodiment 392: The oral composition of embodiment 391, wherein the omega-3 comprising eicosapentaenoic acid (EPA) and docosahexaenoic acid (DHA) is 2:1 EPA:DHA at a dose of 200 mg.

Embodiment 393: The oral composition of embodiment 383, wherein the at least one anti-inflammatory agent is oxindole alkaloids, and wherein the oxindole alkaloids is Unicaria tomentosa (Cat's claw) bark extract with 3% oxindole alkaloids at a dose of between 50 mg and 1200 mg.

Embodiment 394: The oral composition of embodiment 393, wherein the dose of Unicaria tomentosa (Cat's claw) bark extract with 3% oxindole alkaloids is 250 mg.

Embodiment 395: The oral composition of embodiment 383, wherein the at least one anti-inflammatory agent is pterostilbene at a dose of between 10 mg and 250 mg.

Embodiment 396: The oral composition of embodiment 395, wherein the dose of pterostilbene is 50 mg.

Embodiment 397: The oral composition of embodiment 383, wherein the at least one anti-inflammatory agent is resveratrol, and wherein the resveratrol is resveratrol root extract at a dose of between 5 mg and 2000 mg.

Embodiment 398: The oral composition of embodiment 397, wherein the dose of resveratrol root extract is 100 mg.

Embodiment 399: The oral composition of embodiment 383, wherein the at least one anti-inflammatory agent is superoxide dismutase, and wherein the superoxide dismutase is 14000 IU/g superoxide dismutase (SOD) at a dose of between 1 mg and 2000 mg.

Embodiment 400: The oral composition of embodiment 399, wherein the dose of 14000 IU/g superoxide dismutase (SOD) is 75 mg.

Embodiment 401: The oral composition of embodiment 324, wherein the at least one antioxidant is one or more of alpha lipoic acid, astaxanthin, Boswellia serrata extract, coenzyme Q10, Curcuma longa extract, cysteine, dandelion root extract, epigallocatechin gallate, glucoraphanin, glutathione, grape seed extract, hops extract, micro pyrroloquinoline quinone, polyphenols, punicalagin, quercetin, rosemary extract, Schisandra berry extract, shilajit extract, and/or silymarin.

Embodiment 402: The oral composition of embodiment 401, wherein the at least one antioxidant is alpha lipoic acid at a dose of between 5 mg and 1800 mg.

Embodiment 403: The oral composition of embodiment 402, wherein the dose of alpha lipoic acid is 100 mg.

Embodiment 404: The oral composition of embodiment 401, wherein the at least one antioxidant is astaxanthin at a dose of between 1 mg and 20 mg.

Embodiment 405: The oral composition of embodiment 404, wherein the dose of astaxanthin is 5 mg.

Embodiment 406: The oral composition of embodiment 401, wherein the at least one antioxidant is Boswellia serrata extract at a dose of between 5 mg and 7000 mg.

Embodiment 407: The oral composition of embodiment 406, wherein the dose of Boswellia serrata extract is 1000 mg.

Embodiment 408: The oral composition of embodiment 401, wherein the at least one antioxidant is coenzyme Q10, and wherein the coenzyme Q10 is coenzyme Q10 blend from sunflower lecithin at a dose of between 10 mg and 1000 mg.

Embodiment 409: The oral composition of embodiment 408, wherein the dose of coenzyme Q10 blend from sunflower lecithin is 50 mg.

Embodiment 410: The oral composition of embodiment 401, wherein the at least one antioxidant is Curcuma longa extract at a dose of between 50 mg and 5000 mg.

Embodiment 411: The oral composition of embodiment 410, wherein the dose of Curcuma longa extract is 250 mg.

Embodiment 412: The oral composition of embodiment 401, wherein the at least one antioxidant is cysteine, and wherein the cysteine is N-Acetyl-L-Cysteine (NAC) at a dose of between 5 mg and 2000 mg.

Embodiment 413: The oral composition of embodiment 412, wherein the dose of NAC is 100 mg.

Embodiment 414: The oral composition of embodiment 401, wherein the at least one antioxidant is dandelion root extract at a dose of between 20 mg and 1000 mg.

Embodiment 415: The oral composition of embodiment 414, wherein the dose of dandelion root extract is 250 mg.

Embodiment 416: The oral composition of embodiment 401, wherein the at least one antioxidant is epigallocatechin gallate (EGCG), and wherein the EGCG is Camellia sinensis leaf extract with 50% EGCG at a dose of between 10 mg and 1000 mg.

Embodiment 417: The oral composition of embodiment 416, wherein the dose of Camellia sinensis leaf extract with 50% EGCG is 100 mg.

Embodiment 418: The oral composition of embodiment 401, wherein the at least one antioxidant is Ginkgo biloba, and wherein the Ginkgo biloba is Ginkgo biloba leaf extract with 24% Ginkoflavonglycosides/6% Terpene Lactones at a dose of between 30 mg and 350 mg.

Embodiment 419: The oral composition of embodiment 418, wherein the dose of Ginkgo biloba leaf extract with 24% Ginkoflavonglycosides/6% Terpene Lactones is 120 mg.

Embodiment 420: The oral composition of embodiment 401, wherein the at least one antioxidant is glucoraphanin, and wherein the glucoraphanin is broccoli seed extract with 13% glucoraphanin at a dose of between 25 mg and 300 mg.

Embodiment 421: The oral composition of embodiment 420, wherein the dose of broccoli seed extract with 13% glucoraphanin is 75 mg.

Embodiment 422: The oral composition of embodiment 401, wherein the at least one antioxidant is glutathione, and wherein the glutathione is reduced L-glutathione at a dose of between 50 mg and 1000 mg.

Embodiment 423: The oral composition of embodiment 422, wherein the dose of reduced L-glutathione is 100 mg.

Embodiment 424: The oral composition of embodiment 401, wherein the at least one antioxidant is grape seed extract at a dose of between 5 mg and 1000 mg.

Embodiment 425: The oral composition of embodiment 424, wherein the dose of grape seed extract is 100 mg.

Embodiment 426: The oral composition of embodiment 401, wherein the at least one antioxidant is hops extract at a dose of between 25 mg and 1000 mg.

Embodiment 427: The oral composition of embodiment 426, wherein the dose of hops extract is 125 mg.

Embodiment 428: The oral composition of embodiment 401, wherein the at least one antioxidant is micro pyrroloquinoline quinone at a dose of between 1 mg and 800 mg.

Embodiment 429: The oral composition of embodiment 428, wherein the dose of micro pyrroloquinoline quinone is 10 mg.

Embodiment 430: The oral composition of embodiment 401, wherein the at least one antioxidant is polyphenols, and wherein the polyphenols is artichoke flower extract containing 3% polyphenols at a dose of between 20 mg and 1000 mg.

Embodiment 431: The oral composition of embodiment 430, wherein the dose of artichoke flower extract containing 3% polyphenols is 100 mg.

Embodiment 432: The oral composition of embodiment 401, wherein the at least one antioxidant is punicalagins, and wherein the punicalagins is Punica granatum fruit extract containing 30% punicalagins at a dose of between 10 mg and 1500 mg.

Embodiment 433: The oral composition of embodiment 432, wherein the dose of Punica granatum fruit extract containing 30% punicalagins is 250 mg.

Embodiment 434: The oral composition of embodiment 401, wherein the at least one antioxidant is quercetin at a dose of between 10 mg and 2000 mg.

Embodiment 435: The oral composition of embodiment 434, wherein the dose of quercetin is 500 mg.

Embodiment 436: The oral composition of embodiment 401, wherein the at least one antioxidant is rosemary extract at a dose of between 5 mg and 750 mg.

Embodiment 437: The oral composition of embodiment 436, wherein the dose of rosemary extract is 100 mg.

Embodiment 438: The oral composition of embodiment 401, wherein the at least one antioxidant is Schisandra berry extract at a dose of between 20 mg and 3000 mg.

Embodiment 439: The oral composition of embodiment 438, wherein the dose of Schisandra berry extract is 100 mg.

Embodiment 440: The oral composition of embodiment 401, wherein the at least one antioxidant is shilajit extract at a dose of between 20 mg and 1500 mg.

Embodiment 441: The oral composition of embodiment 440, wherein the dose of shilajit extract is 100 mg.

Embodiment 442: The oral composition of embodiment 401, wherein the at least one antioxidant is silymarin, and wherein the silymarin is Silybum marianumshila extract containing 80% silymarin at a dose of between 30 mg and 2400 mg.

Embodiment 443: The oral composition of embodiment 442, wherein the dose of Silybum marianumshila extract containing 80% silymarin is 100 mg.

Embodiment 444: The oral composition of embodiment 324, wherein the at least one amino acid is one or more of alanine, arginine, glutamine, glycine, lysine, methionine, ornithine, phosphatidylserine, proline, tyrosine, taurine, theanine, and/or trimethyl glycine.

Embodiment 445: The oral composition of embodiment 444, wherein the at least one amino acid is alanine, and wherein the alanine is L-alanine at a dose of between 50 mg and 1000 mg.

Embodiment 446: The oral composition of embodiment 445, wherein the dose of L-alanine is 175 mg.

Embodiment 447: The oral composition of embodiment 444, wherein the at least one amino acid is arginine, and wherein the arginine is L-arginine hydrochloride at a dose of between 5 mg and 3000 mg.

Embodiment 448: The oral composition of embodiment 447, wherein the dose of L-arginine hydrochloride is 100 mg.

Embodiment 449: The oral composition of embodiment 444, wherein the at least one amino acid is L-glutamine at a dose of between 1 mg and 3000 mg.

Embodiment 450: The oral composition of embodiment 449, wherein the dose of L-glutamine is 1250 mg.

Embodiment 451: The oral composition of embodiment 444, wherein the at least one amino acid is L-glycine at a dose of between 1 mg and 6000 mg.

Embodiment 452: The oral composition of embodiment 451, wherein the dose of L-glycine is 300 mg.

Embodiment 453: The oral composition of embodiment 444, wherein the at least one amino acid is lysine, and wherein the lysine is L-lysine hydrochloride at a dose of between 5 mg and 2000 mg.

Embodiment 454: The oral composition of embodiment 453, wherein the dose of L-lysine hydrochloride is 500 mg.

Embodiment 455: The oral composition of embodiment 444, wherein the at least one amino acid is methionine, and wherein the methionine is L-methionine at a dose of between 5 mg and 1500 mg.

Embodiment 456: The oral composition of embodiment 455, wherein the dose of L-methionine is 70 mg.

Embodiment 457: The oral composition of embodiment 444, wherein the at least one amino acid is ornithine, and wherein the ornithine is L-ornithine hydrochloride at a dose of between 1 mg and 1000 mg.

Embodiment 458: The oral composition of embodiment 457, wherein the dose of L-ornithine hydrochloride is 50 mg.

Embodiment 459: The oral composition of embodiment 444, wherein the at least one amino acid is phosphatidylserine at a dose of between 10 mg and 2000 mg.

Embodiment 460: The oral composition of embodiment 459, wherein the dose of phosphatidylserine is 100 mg.

Embodiment 461: The oral composition of embodiment 444, wherein the at least one amino acid is proline, and wherein the proline is L-proline at a dose of between 5 mg and 1000 mg.

Embodiment 462: The oral composition of embodiment 461, wherein the dose of L-proline is 250 mg.

Embodiment 463: The oral composition of embodiment 444, wherein the at least one amino acid is tyrosine, and wherein the tyrosine is L-tyrosine at a dose of between 5 mg and 1000 mg.

Embodiment 464: The oral composition of embodiment 463, wherein the dose of L-tyrosine is 50 mg.

Embodiment 465: The oral composition of embodiment 444, wherein the at least one amino acid is taurine at a dose of between 10 mg and 2000 mg.

Embodiment 466: The oral composition of embodiment 465, wherein the dose of taurine is 100 mg.

Embodiment 467: The oral composition of embodiment 444, wherein the at least one amino acid is theanine, and wherein the theanine is L-theanine at a dose of between 5 mg and 2500 mg.

Embodiment 468: The oral composition of embodiment 467, wherein the dose of L-theanine is 100 mg.

Embodiment 469: The oral composition of embodiment 444, wherein the at least one amino acid is trimethyl glycine at a dose of between 10 mg and 1500 mg.

Embodiment 470: The oral composition of embodiment 469, wherein the dose of trimethyl glycine is 1000 mg.

Embodiment 471: The oral composition of embodiment 324, wherein the at least one saccharide is one or more of larch arabinogalactan, beta glucan, and/or ribose.

Embodiment 472: The oral composition of embodiment 471, wherein the at least one saccharide is larch arabinogalactan at a dose of between 50 mg and 2000 mg.

Embodiment 473: The oral composition of embodiment 472, wherein the dose of larch arabinogalactan is 500 mg.

Embodiment 474: The oral composition of embodiment 471, wherein the at least one saccharide is beta glucan, and wherein the beta glucan is 85% 1,3 beta glucan at a dose of between 7.5 mg and 1000 mg.

Embodiment 475: The oral composition of embodiment 474, wherein the dose of 85% 1,3 beta glucan is 500 mg.

Embodiment 476: The oral composition of embodiment 471, wherein the at least one saccharide is ribose, and wherein the ribose is D-ribose at a dose of between 50 mg and 5000 mg.

Embodiment 477: The oral composition of embodiment 476, wherein the dose of D-ribose is 1000 mg.

Embodiment 478: The oral composition of embodiment 324, wherein the at least one herb is Bacopa monnieri, Berberis aristata, bitter melon extract, Citrus bergamia, gingerol, Hericium erinaceus, and/or Withania somnifera.

Embodiment 479: The oral composition of embodiment 478, wherein the at least one herb is Bacopa monnieri extract at a dose of between 50 mg and 500 mg.

Embodiment 480: The oral composition of embodiment 479, wherein the dose of Bacopa monnieri extract is 100 mg.

Embodiment 481: The oral composition of embodiment 478, wherein the at least one herb is Berberis aristata root extract, and wherein the Berberis aristata root extract is Berberis aristata root extract with 97% Berberine HCl at a dose of between 20 mg and 2000 mg.

Embodiment 482: The oral composition of embodiment 481, wherein the dose of Berberis aristata root extract with 97% Berberine HCl is 500 mg.

Embodiment 483: The oral composition of embodiment 478, wherein the at least one herb is bitter melon extract at a dose of between 50 mg and 1000 mg.

Embodiment 484: The oral composition of embodiment 483, wherein the dose of bitter melon extract is 100 mg.

Embodiment 485: The oral composition of embodiment 478, wherein the at least one herb is Citrus bergamia extract, and wherein the Citrus bergamia extract is Citrus bergamia with 40% Polyphenol Fraction at a dose of between 1 mg and 2600 mg.

Embodiment 486: The oral composition of embodiment 485, wherein the dose of Citrus bergamia with 40% Polyphenol Fraction is 500 mg.

Embodiment 487: The oral composition of embodiment 478, wherein the at least one herb is gingerols, and wherein the gingerols is Ginger rhizome extract containing 5% gingerols at a dose of between 10 mg and 1000 mg.

Embodiment 488: The oral composition of embodiment 487, wherein the dose of Ginger rhizome extract containing 5% gingerols is 200 mg.

Embodiment 489: The oral composition of embodiment 478, wherein the at least one herb is Hericium erinaceus extract at a dose of between 1 mg and 3000 mg.

Embodiment 490: The oral composition of embodiment 489, wherein the dose of Hericium erinaceus extract is 100 mg.

Embodiment 491: The oral composition of embodiment 478, wherein the at least one herb is Withania somnifera root extract at a dose of between 50 mg and 2000 mg.

Embodiment 492: The oral composition of embodiment 491, wherein the dose of Withania somnifera root extract is 100 mg.

Embodiment 493: A combination of one or more therapeutic plasma exchange (TPE) sessions and an oral composition for use in removing toxins from a patient who has had chronic exposure to the toxins.

Embodiment 494: The combination for use in removing toxins of embodiment 493, wherein each TPE session is performed with 0.8 to 1.5 estimated plasma volume (EPV).

Embodiment 495: The combination for use in removing toxins of embodiment 494, wherein each TPE session is performed with 5% to 10% albumin.

Embodiment 496: The combination for use in removing toxins of embodiment 495, wherein each TPE session is performed with an anticoagulant.

Embodiment 497: The combination for use in removing toxins of embodiment 496, wherein the anticoagulant is citrate or heparin.

Embodiment 498: The combination for use in removing toxins of embodiment 493 further comprising administration of a metal chelator with the one or more TPE sessions.

Embodiment 499: The combination for use in removing toxins of embodiment 498, wherein the metal chelator is dimercaptosuccinic acid (DMSA), sodium 2,3 dimercaptopropanel sulphonate, ethylenediaminetetraacetic acid (EDTA), sodium calcium edetate, D-penicillamine, or N-acetyl-DL-penicillamine.

Embodiment 500: The combination for use in removing toxins of embodiment 499, wherein the metal chelator is DMSA, and wherein the DMSA is administered at a dose of between 50 mg and 2000 mg.

Embodiment 501: The combination for use in removing toxins of embodiment 500, wherein DMSA is administered at a dose of 250 mg daily for 3 days a week.

Embodiment 502: The combination for use in removing toxins of embodiment 493, wherein the oral composition is administered daily after the first TPE session.

Embodiment 503: The combination for use in removing toxins of embodiment 493, wherein the patient has atherosclerosis.

Embodiment 504: The combination for use in removing toxins of embodiment 503, wherein the combination is capable of reducing the level or slowing the progression of atherosclerosis in the patient.

Embodiment 505: The combination for use in removing toxins of embodiment 503 further comprising administration of at least one cholesterol-reducing agent after the first TPE session.

Embodiment 506: The combination for use in removing toxins of embodiment 505, wherein the at least one cholesterol-reducing agent has not been administered prior to commencing the first TPE session.

Embodiment 507: The combination for use in removing toxins of embodiment 506, wherein the at least one cholesterol-reducing agent is beta-cyclodextrin, and wherein beta-cyclodextrin is administered rectally at a dose of between 8 g and 16 g once a day for 6 months after the first TPE session.

Embodiment 508: The combination for use in removing toxins of embodiment 505, wherein the at least one cholesterol-reducing agent is administered after the first TPE session.

Embodiment 509: The combination for use in removing toxins of embodiment 493, wherein the patient has declined cognitive function.

Embodiment 510: The combination for use in removing toxins of embodiment 509, wherein the combination is capable of improving cognitive function in the patient.

Embodiment 511: The combination for use in removing toxins of embodiment 493, wherein the combination is capable of improving immune function in the patient.

Embodiment 512: The combination for use in removing toxins of embodiment 493, wherein the patient has early cancer development.

Embodiment 513: The combination for use in removing toxins of any one of embodiments 510 to 512 further comprising administration of low dose naltrexone.

Embodiment 514: The combination for use in removing toxins of embodiment 513, wherein naltrexone is administered at a dose of between 1 mg and 20 mg.

Embodiment 515: The combination for use in removing toxins of embodiment 514, wherein low dose naltrexone is taken orally after the first TPE session, wherein the dose of naltrexone is 1.5 mg once daily for 2 weeks, wherein the dose of naltrexone is thereafter increased to 3.0 mg once daily for weeks, and wherein the dose of naltrexone is thereafter increased to 4.5 mg once daily for up to 52 weeks.

Embodiment 516: The combination for use in removing toxins of embodiment 510 further comprising administration of one or more neuropeptides prior to the one or more TPE sessions.

Embodiment 517: The combination for use in removing toxins of embodiment 516, wherein the neuropeptide is adrenocorticotropin, a tetrapeptide having the amino acid sequence of AEDG (SEQ ID NO: 1), a heptapeptide having the amino acid sequence of TKPRPGP (SEQ ID NO: 2), a tripeptide having the amino acid sequence of EDR, a tripeptide having the amino acid sequence of KED, fibroblast growth loop peptide (FGL), brain-derived neurotrophic factor (BDNF), glial cell line-derived neurotrophic factor (GDNF), nerve growth factor (NGF), ciliary neurotrophic factor (CNTF), hypocretin-1, low molecular peptides with a molecular weight up to 5000 Da isolated from the brain tissues of calves, or low molecular peptides with a molecular weight up to 10000 Da isolated from the vascular (aorta) tissue of calves, or a combination thereof.

Embodiment 518: The combination for use in removing toxins of embodiment 517, wherein the neuropeptide is adrenocorticotropin (4-10).

Embodiment 519: The combination for use in removing toxins of embodiment 518, wherein the adrenocorticotropin (4-10) is administered via intranasal spray at a dose of between 1 mcg and 2000 mcg.

Embodiment 520: The combination for use in removing toxins of embodiment 517, wherein the neuropeptide is a tetrapeptide having the amino acid sequence of AEDG (SEQ ID NO: 1).

Embodiment 521: The combination for use in removing toxins of embodiment 520, wherein the tetrapeptide is administered via subcutaneous injection or oral spray at a dose of between 0.1 mg and 50 mg.

Embodiment 522: The combination for use in removing toxins of any one of embodiments 504, 510, or 511, wherein the one or more TPE sessions further comprises administration of exosomes, mesenchymal stem cells (MSCs), or a combination thereof after at least one TPE session but no more than two consecutive TPE sessions.

Embodiment 523: The combination for use in removing toxins of embodiment 522, wherein the MSCs are derived from umbilical cord.

Embodiment 524: The combination for use in removing toxins of embodiment 522, wherein the exosomes are derived from umbilical cord.

Embodiment 525: The combination for use in removing toxins of embodiment 522, wherein the exosomes, mesenchymal stem cells (MSCs), or a combination thereof is administered after at least one TPE session but no more than two consecutive TPE sessions.

Embodiment 526: The combination for use in removing toxins of embodiment 512 further comprising administration of at least one anti-inflammatory compound, one anti-fungal compound, or a combination thereof.

Embodiment 527: The combination for use in removing toxins of embodiment 526 further comprising administration of thymosin al, met enkephalin, methylene blue, nystatin, diflucan, colloidal silver, mebendazole, or a combination thereof.

Embodiment 528: The combination for use in removing toxins of embodiment 527, wherein thymosin al is injected subcutaneously at a dose of between 0.5 mg and 3 mg once a day for 1 to 52 weeks.

Embodiment 529: The combination for use in removing toxins of embodiment 528, wherein thymosin al is injected subcutaneously at a dose of 1.6 mg once a day for 1 to 52 weeks.

Embodiment 530: The combination for use in removing toxins of embodiment 527, wherein met enkephaline is injected subcutaneously at a dose of between 0.5 mg and 3 mg once a day for 1 to 52 weeks.

Embodiment 531: The combination for use in removing toxins of embodiment 530, wherein met enkephaline is injected subcutaneously at a dose of 1.5 mg once a day for 1 to 52 weeks.

Embodiment 532: The combination for use in removing toxins of embodiment 527, wherein methylene blue is taken orally at a dose of between 1 mg and 100 mg once a day for 1 to 52 weeks.

Embodiment 533: The combination for use in removing toxins of embodiment 532, wherein methylene blue is taken orally at a dose of 50 mg once a day for 1 to 52 weeks.

Embodiment 534: The combination for use in removing toxins of embodiment 527, wherein nystatin liquid is taken orally at a dose of between 10,000 units and 4,000,000 units four times a day for 1 to 52 weeks.

Embodiment 535: The combination for use in removing toxins of embodiment 534, wherein nystatin liquid is taken orally at a dose of 500,000 units four times a day for 1 to 52 weeks.

Embodiment 536: The combination for use in removing toxins of embodiment 527, wherein diflucan is taken orally at a dose of between 50 mg and 200 mg once a day for 1 to 52 weeks.

Embodiment 537: The combination for use in removing toxins of embodiment 536, wherein diflucan is taken orally at a dose of 100 mg once a day for 1 to 52 weeks.

Embodiment 538: The combination for use in removing toxins of embodiment 527, wherein colloidal silver 2000 ppm is taken orally at a dose of between 1 mL and 100 mL four times a day for 1 to 52 weeks.

Embodiment 539: The combination for use in removing toxins of embodiment 538, wherein colloidal silver 2000 ppm is taken orally at a dose of 10 mL four times a day for 1 to 52 weeks.

Embodiment 540: The combination for use in removing toxins of embodiment 527, wherein mebendazole is taken orally at a dose of between 50 mg and 200 mg once a day for 1 to 52 weeks.

Embodiment 541: The combination for use in removing toxins of embodiment 540, wherein mebendazole is taken orally at a dose of 200 mg once a day for 1 to 52 weeks.

Embodiment 542: The combination for use in removing toxins of embodiment 493, wherein the one or more TPE sessions further comprises administration of immunoglobulin after each TPE session.

Embodiment 543: The combination for use in removing toxins of embodiment 542, wherein the immunoglobulin is administered via IV or subcutaneous infusion at a dose of between 1 g and 20 g.

Embodiment 544: The combination for use in removing toxins of embodiment 493, wherein between 3 and 8 TPE sessions are performed and each TPE session is performed between 1 and 6 weeks after the preceding TPE session.

Embodiment 545: The combination for use in removing toxins of embodiment 493 further comprising removing plasma at least once every 1 to 20 weeks after the last TPE session.

Embodiment 546: The combination for use in removing toxins of embodiment 545, wherein the plasma is removed once every 1, 2, 3, 4, 5, or up to every 20 weeks after each TPE session but plasma is not removed more than once a week for 3 weeks in a row.

Embodiment 547: The combination for use in removing toxins of embodiment 493, wherein the level of one or more toxins is reduced in the patient.

Embodiment 548: The combination for use in removing toxins of embodiment 547, wherein the one or more toxins is a heavy metal, herbicide, pesticide, phenol, phthalate, volatile organic compound, mycotoxin, per- and/or polyfluorinated substance (PFAS), microplastic, or a combination thereof.

Embodiment 549: The combination for use in removing toxins of embodiment 493, wherein the combination is capable of increasing the level of one or more biomarkers indicating an improvement in immune response in the patient.

Embodiment 550: The combination for use in removing toxins of embodiment 549, wherein the one or more biomarkers indicating an improvement in immune response is CD3, CD4, CD19, CD56, or a combination thereof.

Embodiment 551: The combination for use in removing toxins of embodiment 493, wherein the combination is capable of reducing the level of one or more biomarkers indicating a suppression of inflammation in the patient.

Embodiment 552: The combination for use in removing toxins of embodiment 551, wherein the one or more biomarkers indicating suppression of inflammation is uric acid, hs-C reactive protein, interleukin-8, tumor necrosis factor alpha, or a combination thereof.

Embodiment 553: The combination for use in removing toxins of embodiment 493, wherein the combination is capable of increasing the level of one or more biomarkers associated with longevity in the patient.

Embodiment 554: The combination for use in removing toxins of embodiment 553, wherein the one or more biomarkers associated with longevity is Klotho protein, NAD intracellular, or a combination thereof.

Embodiment 555: The combination for use in removing toxins of embodiment 493, wherein the combination is capable of reducing the level of one or more biomarkers associated with oxidative stress in the patient.

Embodiment 556: The combination for use in removing toxins of embodiment 555, wherein the one or more biomarkers associated with oxidative stress is 8-oxoguanine, myeloperoxidase, or a combination thereof.

Embodiment 557: The combination for use in removing toxins of embodiment 493, wherein the combination is capable of increasing the level of one or more biomarkers associated with cellular senescence in the patient.

Embodiment 558: The combination for use in removing toxins of embodiment 557, wherein the one or more biomarkers associated with cellular senescence is SA-β-galactosidase.

Embodiment 559: The combination for use in removing toxins of embodiment 493, wherein each TPE session further comprises:
(i) administration of an intravenous (IV) composition after performing TPE; and
(ii) administration of a glutathione solution.

Embodiment 560: The combination for use in removing toxins of embodiment 559, wherein between 300 mL and 350 mL of the IV composition is administered.

Embodiment 561: The combination for use in removing toxins of embodiment 560, wherein the IV composition has an osmolality of between 230 mOsm to 300 mOsm.

Embodiment 562: The combination for use in removing toxins of embodiment 559, wherein the glutathione solution is administered by infusion 2 to 3 minutes after administration of the intravenous (IV) composition.

Embodiment 563: The combination for use in removing toxins of embodiment 562, wherein the glutathione solution is administered at a dose of between 1 mg and 5000 mg.

Embodiment 564: The combination for use in removing toxins of embodiment 563, wherein the glutathione solution is administered at a dose of 1000 mg.

Embodiment 565: The combination for use in removing toxins of embodiment 559, wherein the IV composition comprises:
(a) at least one mineral;
(b) at least one vitamin; and
(c) at least one amino acid.

Embodiment 566: The combination for use in removing toxins of embodiment 565, wherein the IV composition comprises the mineral magnesium; the vitamins ascorbic acid, dexpanthenol, hydroxocobalamin, niacin, pyridoxine, riboflavin, and thiamine; and the amino acids carnitine and taurine.

Embodiment 567: The combination for use in removing toxins of embodiment 565, wherein the at least one mineral is one or more of calcium, magnesium, molybdenum, potassium, selenium, and/or zinc.

Embodiment 568: The combination for use in removing toxins of embodiment 567, wherein the at least one mineral is calcium gluconate or calcium chloride.

Embodiment 569: The combination for use in removing toxins of embodiment 568, wherein the at least one mineral is calcium gluconate administered at a dose of between 1 mg and 5000 mg.

Embodiment 570: The combination for use in removing toxins of embodiment 569, wherein calcium gluconate is administered at a dose of 1000 mg.

Embodiment 571: The combination for use in removing toxins of embodiment 567, wherein the at least one mineral is magnesium chloride or magnesium sulfate.

Embodiment 572: The combination for use in removing toxins of embodiment 567, wherein the at least one mineral is magnesium chloride administered at a dose of between 1 mg and 5000 mg.

Embodiment 573: The combination for use in removing toxins of embodiment 572, wherein magnesium chloride is administered at a dose of 2100 mg.

Embodiment 574: The combination for use in removing toxins of embodiment 567, wherein the at least mineral is molybdenum administered at a dose of between 1 mcg and 500 mcg.

Embodiment 575: The combination for use in removing toxins of embodiment 574, wherein molybdenum is administered at a dose of 250 mcg.

Embodiment 576: The combination for use in removing toxins of embodiment 567, wherein the at least one mineral is potassium chloride administered at a dose of between 1 meq and 40 meq.

Embodiment 577: The combination for use in removing toxins of embodiment 576, wherein potassium chloride is administered at a dose of 6 meq.

Embodiment 578: The combination for use in removing toxins of embodiment 567, wherein the at least one mineral is selenium administered at a dose of between 1 mcg and 800 mcg.

Embodiment 579: The combination for use in removing toxins of embodiment 578, wherein selenium is administered at a dose of 400 mcg.

Embodiment 580: The combination for use in removing toxins of embodiment 567, wherein the at least one mineral is zinc sulfate or zinc chloride.

Embodiment 581: The combination for use in removing toxins of embodiment 580, wherein the at least one mineral is zinc sulfate administered at a dose of between 1 mg and 100 mg.

Embodiment 582: The combination for use in removing toxins of embodiment 581, wherein zinc sulfate is administered at a dose of 5 mg.

Embodiment 583: The combination for use in removing toxins of embodiment 565, wherein the at least one vitamin is one or more of ascorbic acid, dexpanthenol, hydroxocobalamin, niacin, pyridoxine, riboflavin, and/or thiamine.

Embodiment 584: The combination for use in removing toxins of embodiment 583, wherein the at least one vitamin is ascorbic acid administered at a dose of between 1 mg and 25000 mg.

Embodiment 585: The combination for use in removing toxins of embodiment 584, wherein ascorbic acid is administered at a dose of 15000 mg.

Embodiment 586: The combination for use in removing toxins of embodiment 583, wherein the at least one vitamin is dexpanthenol administered at a dose of between 1 mg and 2000 mg.

Embodiment 587: The combination for use in removing toxins of embodiment 586, wherein dexpanthenol is administered at a dose of 1500 mg.

Embodiment 588: The combination for use in removing toxins of embodiment 583, wherein the at least one vitamin is hydroxocobalamin or methylcobalamin.

Embodiment 589: The combination for use in removing toxins of embodiment 588, wherein the at least one vitamin is hydroxocobalamin administered at a dose of between 1 mg and 1000 mg.

Embodiment 590: The combination for use in removing toxins of embodiment 589, wherein hydroxocobalamin is administered at a dose of 6 mg.

Embodiment 591: The combination for use in removing toxins of embodiment 583, wherein the at least one vitamin is niacin or niacinamide.

Embodiment 592: The combination for use in removing toxins of embodiment 591, wherein the at least one vitamin is niacin administered at a dose of between 1 mg and 1000 mg.

Embodiment 593: The combination for use in removing toxins of embodiment 592, wherein niacin is administered at a dose of 300 mg.

Embodiment 594: The combination for use in removing toxins of embodiment 583, wherein the at least one vitamin is pyridoxine administered at a dose of between 1 mg and 1000 mg.

Embodiment 595: The combination for use in removing toxins of embodiment 594, wherein pyridoxine is administered at a dose of 100 mg.

Embodiment 596: The combination for use in removing toxins of embodiment 583, wherein the at least one vitamin is riboflavin administered at a dose of between 1 mg and 100 mg.

Embodiment 597: The combination for use in removing toxins of embodiment 596, wherein riboflavin is administered at a dose of 6 mg.

Embodiment 598: The combination for use in removing toxins of embodiment 583, wherein the at least one vitamin is thiamine administered at a dose of between 1 mg and 1000 mg.

Embodiment 599: The combination for use in removing toxins of embodiment 598, wherein thiamine is administered at a dose of 300 mg.

Embodiment 600: The combination for use in removing toxins of embodiment 583, wherein the at least one amino acid is carnitine and/or taurine.

Embodiment 601: The combination for use in removing toxins of embodiment 600, wherein the at least one amino acid is L-carnitine administered at a dose of between 1 mg and 2000 mg.

Embodiment 602: The combination for use in removing toxins of embodiment 601, wherein L-carnitine is administered at a dose of 1000 mg.

Embodiment 603: The combination for use in removing toxins of embodiment 600, wherein the at least one amino acid is taurine administered at a dose of between 1 mg and 5000 mg.

Embodiment 604: The combination for use in removing toxins of embodiment 603, wherein taurine is administered at a dose of 400 mg.

Embodiment 605: The combination for use in removing toxins of embodiment 493, wherein the oral composition comprises:
(a) at least one vitamin;
(b) at least one mineral;
(c) at least one anti-inflammatory agent;
(d) at least one antioxidant;
(e) at least one amino acid;
(f) at least one saccharide; and
(g) at least one herb.

Embodiment 606: The combination for use in removing toxins of embodiment 605, wherein the oral composition further comprises at least one protein.

Embodiment 607: The combination for use in removing toxins of embodiment 605, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, Schisandra berry extract, grape seed extract, micro pyrroloquinoline quinone, alpha lipoic acid, boswellin, and dandelion root extract; the amino acids arginine, glutamine, proline, and tyrosine; the saccharides larch arabinogalactan, ribose, and beta glucan; and the herbs gingerol and bitter melon extract.

Embodiment 608: The combination for use in removing toxins of embodiment 605 further comprising vinpocetine and Panax ginseng, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, schisandra berry extract, grape seed extract, micro pyrroloquinoline quinone, alpha lipoic acid, boswellin, dandelion root extract, and Ginkgo biloba; the amino acids arginine, glutamine, proline, tyrosine, phosphatidylserine, and theanine; the saccharides larch arabinogalactan, ribose, and beta glucan; and the herbs gingerol, bitter melon extract, Hericium erinaceus, Withania somnifera, and Bacopa monnieri.

Embodiment 609: The combination for use in removing toxins of embodiment 605, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, schisandra berry extract, grape seed extract, micro pyrroloquinoline quinone, alpha lipoic acid, boswellin, and dandelion root extract; the amino acids arginine, glutamine, proline, and tyrosine; the saccharides larch arabinogalactan, ribose, and beta glucan; and the herbs gingerol, bitter melon extract, Berberis aristata, and Citrus bergamia.

Embodiment 610: The combination for use in removing toxins of any one of embodiments 607 to 609, wherein the oral composition further comprises at least one protein.

Embodiment 611: The combination for use in removing toxins of either embodiment 606 or embodiment 607, wherein the protein is rice protein.

Embodiment 612: The combination for use in removing toxins of embodiment 605, wherein the oral composition further comprises at least one other component.

Embodiment 613: The combination for use in removing toxins of embodiment 612, wherein the at least one other component is vinpocentine or Panax ginseng.

Embodiment 614: The combination for use in removing toxins of embodiment 612, wherein the at least one other component is vinpocentine administered at a dose of between 1 mg and 100 mg.

Embodiment 615: The combination for use in removing toxins of embodiment 612, wherein vinpocentine is administered at a dose of 30 mg.

Embodiment 616: The combination for use in removing toxins of embodiment 612, wherein the at least one other component is Panax ginseng, and wherein the Panax ginseng is Panax ginseng 5% Powdered Extract administered at a dose of between 1 mg and 2000 mg.

Embodiment 617: The combination for use in removing toxins of embodiment 616, wherein Panax ginseng 5% Powdered Extract is administered at a dose of 100 mg.

Embodiment 618: The combination for use in removing toxins of embodiment 605, wherein the at least one vitamin is one or more of biotin, cobalamin, folate, pantothenic acid, pyridoxine, riboflavin, thiamine, vitamin A, vitamin B3, vitamin C, vitamin D3, and/or vitamin E.

Embodiment 619: The combination for use in removing toxins of embodiment 618, wherein the at least one vitamin is biotin administered at a dose of between 15 mcg and 7500 mcg.

Embodiment 620: The combination for use in removing toxins of embodiment 619, wherein biotin is administered at a dose of 50 mcg.

Embodiment 621: The combination for use in removing toxins of embodiment 618, wherein the at least one vitamin is cobalamin, and wherein the cobalamin is methylcobalamin administered at a dose of between 10 mcg and 10000 mcg.

Embodiment 622: The combination for use in removing toxins of embodiment 621, wherein methylcobalamin is administered at a dose of 500 mcg.

Embodiment 623: The combination for use in removing toxins of embodiment 618, wherein the at least one vitamin is folate, and wherein the folate is L-5-methyltetrahydrofolate glucosamine salt administered at a dose of between 10 mcg and 7500 mcg.

Embodiment 624: The combination for use in removing toxins of embodiment 623, wherein L-5-methyltetrahydrofolate glucosamine salt is administered at a dose of 1000 mcg.

Embodiment 625: The combination for use in removing toxins of embodiment 624, wherein the at least one vitamin is pantothenic acid administered at a dose of between 1 mg and 1500 mg.

Embodiment 626: The combination for use in removing toxins of embodiment 625, wherein pantothenic acid is administered at a dose of 100 mg.

Embodiment 627: The combination for use in removing toxins of embodiment 618, wherein the at least one vitamin is pyridoxine administered at a dose of between 1 mg and 300 mg.

Embodiment 628: The combination for use in removing toxins of embodiment 627, wherein pyridoxine is administered at a dose of 15 mg.

Embodiment 629: The combination for use in removing toxins of embodiment 618, wherein the at least one vitamin is riboflavin administered at a dose of between 1 mg and 500 mg.

Embodiment 630: The combination for use in removing toxins of embodiment 629, wherein riboflavin is administered at a dose of 15 mg.

Embodiment 631: The combination for use in removing toxins of embodiment 618, wherein the at least one vitamin is thiamine administered at a dose of between 1 mg and 300 mg.

Embodiment 632: The combination for use in removing toxins of embodiment 631, wherein thiamine is administered at a dose of 1 mg.

Embodiment 633: The combination for use in removing toxins of embodiment 618, wherein the at least one vitamin is vitamin A, and wherein the vitamin A is vitamin A palmitate administered at a dose of between 1 mg and 2000 mg.

Embodiment 634: The combination for use in removing toxins of embodiment 633, wherein vitamin A palmitate is administered at a dose of 1000 mg.

Embodiment 635: The combination for use in removing toxins of embodiment 618, wherein the at least one vitamin is vitamin B3, and wherein the vitamin B3 is niacin and/or nicotinamide riboside chloride.

Embodiment 636: The combination for use in removing toxins of embodiment 635, wherein the at least one vitamin is niacin administered at a dose of between 1 mg and 750 mg.

Embodiment 637: The combination for use in removing toxins of embodiment 636, wherein niacin is administered at a dose of 15 mg.

Embodiment 638: The combination for use in removing toxins of embodiment 635, wherein the at least vitamin is nicotinamide riboside chloride administered at a dose of between 1 mg and 1000 mg.

Embodiment 639: The combination for use in removing toxins of embodiment 638, wherein nicotinamide riboside chloride is administered at a dose of 750 mg.

Embodiment 640: The combination for use in removing toxins of embodiment 618, wherein the at least one vitamin is vitamin C, and wherein the vitamin C is ascorbic acid administered at a dose of between 1 mg and 3000 mg.

Embodiment 641: The combination for use in removing toxins of embodiment 640, wherein ascorbic acid is administered at a dose of 500 mg.

Embodiment 642: The combination for use in removing toxins of embodiment 618, wherein the at least one vitamin is vitamin D3, and wherein the vitamin D3 is cholecalciferol administered at a dose of between 10 IU and 50000 IU.

Embodiment 643: The combination for use in removing toxins of embodiment 642, wherein cholecalciferol is administered at a dose of 2500 IU.

Embodiment 644: The combination for use in removing toxins of embodiment 618, wherein the at least one vitamin is vitamin E, and wherein the vitamin E is mixed tocopherols administered at a dose of between 10 IU and 500 IU.

Embodiment 645: The combination for use in removing toxins of embodiment 644, wherein the mixed tocopherols are administered at a dose of 50 IU.

Embodiment 646: The combination for use in removing toxins of embodiment 605, wherein the at least one mineral is one or more of calcium, chromium, copper, iodine, magnesium, manganese, molybdenum, and/or zinc.

Embodiment 647: The combination for use in removing toxins of embodiment 646, wherein the at least one mineral is calcium, and wherein the calcium is calcium d-glucarate administered at a dose of between 25 mg and 1000 mg.

Embodiment 648: The combination for use in removing toxins of embodiment 647, wherein calcium d-glucarate is administered at a dose of 500 mg.

Embodiment 649: The combination for use in removing toxins of embodiment 646, wherein the at least one mineral is chromium, and wherein the chromium is chromium polynicotinate administered at a dose of between 10 mcg and 1000 mcg.

Embodiment 650: The combination for use in removing toxins of embodiment 649, wherein chromium polynicotinate is administered at a dose of 25 mcg.

Embodiment 651: The combination for use in removing toxins of embodiment 646, wherein the at least one mineral is copper, and wherein the copper is copper gluconate administered at a dose of between 0.1 mg and 10 mg.

Embodiment 652: The combination for use in removing toxins of embodiment 651, wherein copper gluconate is administered at a dose of 1 mg.

Embodiment 653: The combination for use in removing toxins of embodiment 646, wherein the at least one mineral is iodine, and wherein the iodine is potassium iodide administered at a dose of between 10 mcg and 500 mcg.

Embodiment 654: The combination for use in removing toxins of embodiment 653, wherein potassium iodide administered at a dose of 25 mcg.

Embodiment 655: The combination for use in removing toxins of embodiment 646, wherein the at least one mineral is magnesium, and wherein the magnesium is magnesium bisglycinate chelate buffered and/or magnesium glycinate.

Embodiment 656: The combination for use in removing toxins of embodiment 655, wherein the at least one mineral is magnesium bisglycinate chelate buffered administered at a dose of between 2 mg and 2000 mg.

Embodiment 657: The combination for use in removing toxins of embodiment 656, wherein magnesium bisglycinate chelate buffered is administered at a dose of 100 mg.

Embodiment 658: The combination for use in removing toxins of embodiment 655, wherein the at least one mineral is magnesium glycinate administered at a dose of between 0.5 mg and 20 mg.

Embodiment 659: The combination for use in removing toxins of embodiment 658, wherein magnesium glycinate is administered at a dose of 5 mg.

Embodiment 660: The combination for use in removing toxins of embodiment 646, wherein the at least one mineral is molybdenum, and wherein the molybdenum is molybdenum glycinate chelate administered at a dose of between 1 mcg and 500 mcg.

Embodiment 661: The combination for use in removing toxins of embodiment 660, wherein molybdenum glycinate chelate is administered at a dose of 50 mcg.

Embodiment 662: The combination for use in removing toxins of embodiment 646, wherein the at least one mineral is zinc, and wherein the zinc is zinc bisglycinate chelate administered at a dose of between 1 mg and 50 mg.

Embodiment 663: The combination for use in removing toxins of embodiment 662, wherein zinc bisglycinate chelate is administered at a dose of 5 mg.

Embodiment 664: The combination for use in removing toxins of embodiment 605, wherein the at least one anti-inflammatory agent is one or more of baicalin, bromelain, Noni fruit extract, omega-3, oxindole alkaloid, pterostilbene, resveratrol, and/or superoxide dismutase.

Embodiment 665: The combination for use in removing toxins of embodiment 664, wherein the at least one anti-inflammatory agent is baicalin, and wherein the baicalin is skullcap root extract containing 30% baicalin administered at a dose of between 25 mg to 1000 mg.

Embodiment 666: The combination for use in removing toxins of embodiment 665, wherein skullcap root extract containing 30% baicalin is administered at a dose of 250 mg.

Embodiment 667: The combination for use in removing toxins of embodiment 664, wherein the at least one anti-inflammatory agent is bromelain, and wherein the bromelain is bromelain 24000 gelatin dissolving units (GDU) administered at a dose of between 1 and 1000 mg.

Embodiment 668: The combination for use in removing toxins of embodiment 667, wherein bromelain 24000 gelatin dissolving units (GDU) is administered at a dose of 50 mg.

Embodiment 669: The combination for use in removing toxins of embodiment 664, wherein the at least one anti-inflammatory agent is Noni fruit extract, and wherein the Noni fruit extract is Noni fruit extract 4:1 administered at a dose of between 1 and 1000 mg.

Embodiment 670: The combination for use in removing toxins of embodiment 669, wherein Noni fruit extract 4:1 is administered at a dose of 50 mg.

Embodiment 671: The combination for use in removing toxins of embodiment 664, wherein the at least one anti-inflammatory agent is omega-3, and wherein the omega-3 is omega-3 comprising eicosapentaenoic acid (EPA) and docosahexaenoic acid (DHA).

Embodiment 672: The combination for use in removing toxins of embodiment 671, wherein the omega-3 comprising eicosapentaenoic acid (EPA) and docosahexaenoic acid (DHA) is 2:1 EPA:DHA administered at a dose of between 25 mg and 3000 mg.

Embodiment 673: The combination for use in removing toxins of embodiment 672, wherein the omega-3 comprising eicosapentaenoic acid (EPA) and docosahexaenoic acid (DHA) is 2:1 EPA:DHA administered at a dose of 200 mg.

Embodiment 674: The combination for use in removing toxins of embodiment 664, wherein the at least one anti-inflammatory agent is oxindole alkaloids, and wherein the oxindole alkaloids is Unicaria tomentosa (Cat's claw) bark extract with 3% oxindole alkaloids administered at a dose of between 50 mg and 1200 mg.

Embodiment 675: The combination for use in removing toxins of embodiment 674, wherein Unicaria tomentosa (Cat's claw) bark extract with 3% oxindole alkaloids is administered at a dose of 250 mg.

Embodiment 676: The combination for use in removing toxins of embodiment 664, wherein the at least one anti-inflammatory agent is pterostilbene administered at a dose of between 10 mg and 250 mg.

Embodiment 677: The combination for use in removing toxins of embodiment 184, wherein pterostilbene is administered at a dose of 50 mg.

Embodiment 186/678: The combination for use in removing toxins of embodiment 664, wherein the at least one anti-inflammatory agent is resveratrol, and wherein the resveratrol is resveratrol root extract administered at a dose of between 5 mg and 2000 mg.

Embodiment 679: The combination for use in removing toxins of embodiment 678, wherein resveratrol root extract is administered at a dose of 100 mg.

Embodiment 680: The combination for use in removing toxins of embodiment 664, wherein the at least one anti-inflammatory agent is superoxide dismutase, and wherein the superoxide dismutase is 14000 IU/g superoxide dismutase (SOD) administered at a dose of between 1 mg and 2000 mg.

Embodiment 681: The combination for use in removing toxins of embodiment 680, wherein 14000 IU/g superoxide dismutase (SOD) is administered at a dose of 75 mg.

Embodiment 682: The combination for use in removing toxins of embodiment 605, wherein the at least one antioxidant is one or more of alpha lipoic acid, astaxanthin, Boswellia serrata extract, coenzyme Q10, Curcuma longa extract, cysteine, dandelion root extract, epigallocatechin gallate, Ginkgo biloba, glucoraphanin, glutathione, grape seed extract, hops extract, micro pyrroloquinoline quinone, polyphenols, punicalagin, quercetin, rosemary extract, schisandra berry extract, shilajit extract, and/or silymarin.

Embodiment 683: The combination for use in removing toxins of embodiment 682, wherein the at least one antioxidant is alpha lipoic acid administered at a dose of between 5 mg and 1800 mg.

Embodiment 684: The combination for use in removing toxins of embodiment 683, wherein alpha lipoic acid is administered at a dose of 100 mg.

Embodiment 685: The combination for use in removing toxins of embodiment 682, wherein the at least one antioxidant is astaxanthin administered at a dose of between 1 mg and 20 mg.

Embodiment 686: The combination for use in removing toxins of embodiment 685, wherein astaxanthin is administered at a dose of 5 mg.

Embodiment 687: The combination for use in removing toxins of embodiment 682, wherein the at least one antioxidant is Boswellia serrata extract administered at a dose of between 5 mg and 7000 mg.

Embodiment 688: The combination for use in removing toxins of embodiment 687, wherein Boswellia serrata extract is administered at a dose of 1000 mg.

Embodiment 689: The combination for use in removing toxins of embodiment 682, wherein the at least one antioxidant is coenzyme Q10, and wherein the coenzyme Q10 is coenzyme Q10 blend from sunflower lecithin administered at a dose of between 10 mg and 1000 mg.

Embodiment 690: The combination for use in removing toxins of embodiment 689, wherein coenzyme Q10 blend from sunflower lecithin is administered at a dose of 50 mg.

Embodiment 691: The combination for use in removing toxins of embodiment 682, wherein the at least one antioxidant is Curcuma longa Extract administered at a dose of between 50 mg and 5000 mg.

Embodiment 692: The combination for use in removing toxins of embodiment 691, wherein Curcuma longa Extract is administered at a dose of 250 mg.

Embodiment 693: The combination for use in removing toxins of embodiment 682, wherein the at least one antioxidant is cysteine, and wherein the cysteine is N-Acetyl-L-Cysteine (NAC) administered at a dose of between 5 mg and 2000 mg.

Embodiment 694: The combination for use in removing toxins of embodiment 693, wherein NAC is administered at a dose of 100 mg.

Embodiment 695: The combination for use in removing toxins of embodiment 682, wherein the at least one antioxidant is dandelion root extract administered at a dose of between 20 mg and 1000 mg.

Embodiment 696: The combination for use in removing toxins of embodiment 695, wherein dandelion root extract is administered at a dose of 250 mg.

Embodiment 697: The combination for use in removing toxins of embodiment 682, wherein the at least one antioxidant is epigallocatechin gallate (EGCG), and wherein the EGCG is Camellia sinensis leaf extract with 50% EGCG administered at a dose of between 10 mg and 1000 mg.

Embodiment 698: The combination for use in removing toxins of embodiment 697, wherein Camellia sinensis leaf extract with 50% EGCG is administered at a dose of 100 mg.

Embodiment 699: The combination for use in removing toxins of embodiment 682, wherein the at least one antioxidant is Ginkgo biloba, and wherein the Ginkgo biloba is Ginkgo biloba leaf extract with 24% Ginkoflavonglycosides/6% Terpene Lactones administered at a dose of between 30 mg and 350 mg.

Embodiment 700: The combination for use in removing toxins of embodiment 699, wherein Ginkgo biloba leaf extract with 24% Ginkoflavonglycosides/6% Terpene Lactones is administered at a dose of 120 mg.

Embodiment 701: The combination for use in removing toxins of embodiment 682, wherein the at least one antioxidant is glucoraphanin, and wherein the glucoraphanin is broccoli seed extract with 13% glucoraphanin administered at a dose of between 25 mg and 300 mg.

Embodiment 702: The combination for use in removing toxins of embodiment 701, wherein broccoli seed extract with 13% glucoraphanin is administered at a dose of 75 mg.

Embodiment 703: The combination for use in removing toxins of embodiment 682, wherein the at least one antioxidant is glutathione, and wherein the glutathione is reduced L-glutathione administered at a dose of between 50 mg and 1000 mg.

Embodiment 704: The combination for use in removing toxins of embodiment 703, wherein reduced L-glutathione administered at a dose of 100 mg.

Embodiment 705: The combination for use in removing toxins of embodiment 682, wherein the at least one antioxidant is grape seed extract administered at a dose of between 5 mg and 1000 mg.

Embodiment 706: The combination for use in removing toxins of embodiment 705, wherein grape seed extract is administered at a dose of 100 mg.

Embodiment 707: The combination for use in removing toxins of embodiment 682, wherein the at least one antioxidant is hops extract administered at a dose of between 25 mg and 1000 mg.

Embodiment 708: The combination for use in removing toxins of embodiment 707, wherein hops extract is administered at a dose of 125 mg.

Embodiment 709: The combination for use in removing toxins of embodiment 682, wherein the at least one antioxidant is micro pyrroloquinoline quinone administered at a dose of between 1 mg and 800 mg.

Embodiment 710: The combination for use in removing toxins of embodiment 709, wherein micro pyrroloquinoline quinone is administered at a dose of 10 mg.

Embodiment 711: The combination for use in removing toxins of embodiment 682, wherein the at least one antioxidant is polyphenols, and wherein the polyphenols are artichoke flower extract containing 3% polyphenols administered at a dose of between 20 mg and 1000 mg.

Embodiment 712: The combination for use in removing toxins of embodiment 711, wherein artichoke flower extract containing 3% polyphenols is administered at a dose of 100 mg.

Embodiment 713: The combination for use in removing toxins of embodiment 682, wherein the at least one antioxidant is punicalagins, and wherein the punicalagins is Punica granatum fruit extract containing 30% punicalagins administered at a dose of between 10 mg and 1500 mg.

Embodiment 714: The combination for use in removing toxins of embodiment 685, wherein Punica granatum fruit extract containing 30% punicalagins is administered at a dose of 250 mg.

Embodiment 715: The combination for use in removing toxins of embodiment 682, wherein the at least one antioxidant is quercetin administered at a dose of between 10 mg and 2000 mg.

Embodiment 716: The combination for use in removing toxins of embodiment 687, wherein quercetin is administered at a dose of 500 mg.

Embodiment 717: The combination for use in removing toxins of embodiment 682, wherein the at least one antioxidant is rosemary extract administered at a dose of between 5 mg and 750 mg.

Embodiment 718: The combination for use in removing toxins of embodiment 689, wherein rosemary extract is administered at a dose of 100 mg.

Embodiment 719: The combination for use in removing toxins of embodiment 682, wherein the at least one antioxidant is Schisandra berry extract administered at a dose of between 20 mg and 3000 mg.

Embodiment 720: The combination for use in removing toxins of embodiment 691, wherein Schisandra berry extract is administered at a dose of 100 mg.

Embodiment 721: The combination for use in removing toxins of embodiment 682, wherein the at least one antioxidant is shilajit extract administered at a dose of between 20 mg and 1500 mg.

Embodiment 722: The combination for use in removing toxins of embodiment 693, wherein shilajit extract is administered at a dose of 100 mg.

Embodiment 723: The combination for use in removing toxins of embodiment 682, wherein the at least one antioxidant is silymarin, and wherein the silymarin is Silybum marianumshila extract containing 80% silymarin administered at a dose of between 30 mg and 2400 mg.

Embodiment 724: The combination for use in removing toxins of embodiment 722, wherein Silybum marianumshila extract containing 80% silymarin is administered at a dose of 100 mg.

Embodiment 725: The combination for use in removing toxins of embodiment 605, wherein the at least one amino acid is one or more of alanine, arginine, glutamine, glycine, lysine, methionine, ornithine, phosphatidylserine, proline, tyrosine, taurine, theanine, and/or trimethyl glycine.

Embodiment 726: The combination for use in removing toxins of embodiment 725, wherein the at least one amino acid is alanine, and wherein the alanine is L-alanine administered at a dose of between 50 mg and 1000 mg.

Embodiment 727: The combination for use in removing toxins of embodiment 726, wherein L-alanine is administered at a dose of 175 mg.

Embodiment 728: The combination for use in removing toxins of embodiment 725, wherein the at least one amino acid is arginine, and wherein the arginine is L-arginine hydrochloride administered at a dose of between 5 mg and 3000 mg.

Embodiment 729: The combination for use in removing toxins of embodiment 728, wherein L-arginine hydrochloride is administered at a dose of 100 mg.

Embodiment 730: The combination for use in removing toxins of embodiment 725, wherein the at least one amino acid is L-glutamine administered at a dose of between 1 mg and 3000 mg.

Embodiment 731: The combination for use in removing toxins of embodiment 730, wherein L-glutamine is administered at a dose of 1250 mg.

Embodiment 732: The combination for use in removing toxins of embodiment 725, wherein the at least one amino acid is L-glycine administered at a dose of between 1 mg and 6000 mg.

Embodiment 733: The combination for use in removing toxins of embodiment 732, wherein L-glycine is administered at a dose of 300 mg.

Embodiment 734: The combination for use in removing toxins of embodiment 725, wherein the at least one amino acid is lysine, and wherein the lysine is L-lysine hydrochloride administered at a dose of between 5 mg and 2000 mg.

Embodiment 735: The combination for use in removing toxins of embodiment 734, wherein L-lysine hydrochloride is administered at a dose of 500 mg.

Embodiment 736: The combination for use in removing toxins of embodiment 725, wherein the at least one amino acid is methionine, and wherein the methionine is L-methionine administered at a dose of between 5 mg and 1500 mg.

Embodiment 737: The combination for use in removing toxins of embodiment 736, wherein L-methionine is administered at a dose of 70 mg.

Embodiment 738: The combination for use in removing toxins of embodiment 725, wherein the at least one amino acid is ornithine, and wherein the ornithine is L-ornithine hydrochloride administered at a dose of between 1 mg and 1000 mg.

Embodiment 739: The combination for use in removing toxins of embodiment 738, wherein L-ornithine hydrochloride is administered at a dose of 50 mg.

Embodiment 740: The combination for use in removing toxins of embodiment 725, wherein the at least one amino acid is phosphatidylserine administered at a dose of between 10 mg and 2000 mg.

Embodiment 741: The combination for use in removing toxins of embodiment 740, wherein phosphatidylserine is administered at a dose of 100 mg.

Embodiment 742: The combination for use in removing toxins of embodiment 725, wherein the at least one amino acid is proline, and wherein the proline is L-proline administered at a dose of between 5 mg and 1000 mg.

Embodiment 743: The combination for use in removing toxins of embodiment 742, wherein L-proline is administered at a dose of 250 mg.

Embodiment 744: The combination for use in removing toxins of embodiment 725, wherein the at least one amino acid is tyrosine, and wherein the tyrosine is L-tyrosine administered at a dose of between 5 mg and 1000 mg.

Embodiment 745: The combination for use in removing toxins of embodiment 744, wherein L-tyrosine is administered at a dose of 50 mg.

Embodiment 746: The combination for use in removing toxins of embodiment 725, wherein the at least one amino acid is taurine administered at a dose of between 10 mg and 2000 mg.

Embodiment 747: The combination for use in removing toxins of embodiment 746, wherein taurine is administered at a dose of 100 mg.

Embodiment 748: The combination for use in removing toxins of embodiment 725, wherein the at least one amino acid is theanine, and wherein the theanine is L-theanine administered at a dose of between 5 mg and 2500 mg.

Embodiment 749: The combination for use in removing toxins of embodiment 748, wherein L-theanine is administered at a dose of 100 mg.

Embodiment 750: The combination for use in removing toxins of embodiment 725, wherein the at least one amino acid is trimethyl glycine administered at a dose of between 10 mg and 1500 mg.

Embodiment 751: The combination for use in removing toxins of embodiment 750, wherein trimethyl glycine is administered at a dose of 1000 mg.

Embodiment 752: The combination for use in removing toxins of embodiment 605, wherein the at least one saccharide is one or more of larch arabinogalactan, beta glucan, and/or ribose.

Embodiment 753: The combination for use in removing toxins of embodiment 752, wherein the at least one saccharide is larch arabinogalactan administered at a dose of between 50 mg and 2000 mg.

Embodiment 754: The combination for use in removing toxins of embodiment 753, wherein larch arabinogalactan is administered at a dose of 500 mg.

Embodiment 755: The combination for use in removing toxins of embodiment 752, wherein the at least one saccharide is beta glucan, and wherein the beta glucan is 85% 1,3 beta glucan administered at a dose of between 7.5 mg and 1000 mg.

Embodiment 756: The combination for use in removing toxins of embodiment 755, wherein 85% 1,3 beta glucan is administered at a dose of 500 mg.

Embodiment 757: The combination for use in removing toxins of embodiment 752, wherein the at least one saccharide is ribose, and wherein the ribose is D-ribose administered at a dose of between 50 mg and 5000 mg.

Embodiment 758: The combination for use in removing toxins of embodiment 757, wherein D-ribose is administered at a dose of 1000 mg.

Embodiment 759: The combination for use in removing toxins of embodiment 605, wherein the at least one herb is Bacopa monnieri, Berberis aristata, bitter melon extract, Citrus bergamia, gingerol, Hericium erinaceus, and/or Withania somnifera.

Embodiment 760: The combination for use in removing toxins of embodiment 759, wherein the at least one herb is Bacopa monnieri extract administered at a dose of between 50 mg and 500 mg.

Embodiment 761: The combination for use in removing toxins of embodiment 760, wherein Bacopa monnieri extract is administered at a dose of 100 mg.

Embodiment 762: The combination for use in removing toxins of embodiment 759, wherein the at least one herb is Berberis aristata root extract, and wherein the Berberis aristata root extract is Berberis aristata root extract with 97% Berberine HCl administered at a dose of between 20 mg and 2000 mg.

Embodiment 763: The combination for use in removing toxins of embodiment 762, wherein Berberis aristata root extract with 97% Berberine HCl is administered at a dose of 500 mg.

Embodiment 764: The combination for use in removing toxins of embodiment 759, wherein the at least one herb is bitter melon extract administered at a dose of between 50 mg and 1000 mg.

Embodiment 765: The combination for use in removing toxins of embodiment 764, wherein bitter melon extract is administered at a dose of 100 mg.

Embodiment 766: The combination for use in removing toxins of embodiment 759, wherein the at least one herb is Citrus bergamia extract, and wherein the Citrus bergamia extract is Citrus bergamia with 40% Polyphenol Fraction administered at a dose of between 1 mg and 2600 mg.

Embodiment 767: The combination for use in removing toxins of embodiment 766, wherein Citrus bergamia with 40% Polyphenol Fraction is administered at a dose of 500 mg.

Embodiment 768: The combination for use in removing toxins of embodiment 759, wherein the at least one herb is gingerols, and wherein the gingerols is Ginger rhizome extract containing 5% gingerols administered at a dose of between 10 mg and 1000 mg.

Embodiment 769: The combination for use in removing toxins of embodiment 768, wherein Ginger rhizome extract containing 5% gingerols is administered at a dose of 200 mg.

Embodiment 770: The combination for use in removing toxins of embodiment 759, wherein the at least one herb is Hericium erinaceus extract administered at a dose of between 1 mg and 3000 mg.

Embodiment 771: The combination for use in removing toxins of embodiment 770, wherein Hericium erinaceus extract is administered at a dose of 100 mg.

Embodiment 772: The combination for use in removing toxins of embodiment 759, wherein the at least one herb is Withania somnifera root extract administered at a dose of between 50 mg and 2000 mg.

Embodiment 773: The combination for use in removing toxins of embodiment 772, wherein Withania somnifera root extract is administered at a dose of 100 mg.

Embodiment 774: Use of one or more therapeutic plasma exchange (TPE) sessions and an oral composition for the manufacture of a medicament for removing toxins from a patient who has had chronic exposure to the toxins.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Methods

### 1. Pre-Treatment and Post-Treatment Tests

i) Combined TPE and TPE only treatments: two laboratory tests were scheduled 2 weeks and 1 week before starting the treatment protocol. Three laboratory tests were scheduled at 2 weeks, 6 weeks, and 6 months after completion of the treatment. For each appointment, patients were asked to fast for 10 hours with water only and stop taking any medications or supplements the morning of the appointment. Additionally, 3 capsules each containing 500 mg dimercaptosuccinic acid (DMSA), which is a metal chelator, were taken orally by the patients before bed the night before the morning of the appointment. Urine was collected during the first morning void, and blood was drawn at the appointment for laboratory tests.

ii) Heart TPE treatment: in heart TPE treatment on patients with atherosclerosis, the patients were additionally subjected to computed tomography (CT) angiogram two weeks to two months before the treatment and 6 weeks and 1 year after completion of the treatment.

iii) Brain TPE treatment: two weeks to two months before the treatment and 6 weeks and 1 year after completion of the treatment, patients were subjected to a central nervous system vital signs (CNS-VS) assessment and a mini mental status exam. Patients were also subjected to an MRI brain scan to evaluate volume of the hippocampus and rule out vascular dementia. One laboratory test was scheduled two weeks before the treatment. Three laboratory tests were scheduled at 6 weeks and 6 months after completion of the treatment.

iv) Cancer TPE treatment: Blood samples were collected from patients before and after cancer TPE treatment for laboratory tests. Magnetic resonance imaging (MRI) in patient with prostate cancer was performed before and after cancer TPE treatment.

v) Immunity TPE treatment: Blood samples were collected from patients before and after cancer TPE treatment for laboratory tests.

### 2. Treatment Protocol

i) Combined TPE and TPE only treatments: the treatment protocol comprised a series of therapeutic plasma exchange (TPE) sessions. At each session, TPE was performed on the patients with 0.8-1.5 estimated plasma volume (EPV) exchange with 5% albumin and saline, with citrate as an anticoagulant. Each session was repeated every 4 weeks, with a complete treatment comprising 3-5 sessions. Some patients donated plasma every 6-12 weeks after all TPE sessions were completed.

In the combined TPE treatment, patients were given additional supplementation as follows: immediately following each TPE session, the patient was given an IV infusion of supplements. About 2 minutes after the IV infusion was completed, 5 mL of 200 mg/mL glutathione was administered to the patients. The IV composition is shown in Table 3. After the first TPE session, patients were supplemented with oral nutrients daily (using an oral composition, the components of which are shown in Table 4). The patients were instructed to take 1 scoop (about 60 g) of powdered oral nutrients in 8 oz. of water orally starting the day after the first session and until the final lab test appointment.

After the first TPE was completed, subcutaneous infusion of 5 g immunoglobulin was administered to the patient in both combined TPE and TPE only treatment groups.

To distinguish from the combined TPE treatment, TPE treatment without IV and oral compositions supplementation was called TPE only treatment or control TPE treatment.

All patients in both groups were also advised to make some lifestyle changes to avoid exposure to toxins including air filtration, water filtration, food selection and preparation, and elimination of household chemical toxins.

ii) Heart TPE treatment: the treatment protocol included the combined TPE treatment protocol described above on patients with atherosclerosis. Additionally, 8 g of beta-cyclodextrin CAVADEX was administered rectally to the patients daily for 6 months after the first TPE treatment session. Additionally, after the first TPE was completed, the patient was administered DMSA orally at 250 mg on Monday through Wednesday every week for 1 to 52 weeks.

The oral compositions taken by this group of patients comprised the components shown in Table 4 plus additional herbs comprising 500 mg Berberis aristata root extract with 97% Berberine HCl and 500 mg Citrus bergamia with 40% Polyphenol Fraction. The treatment protocol included the combined TPE treatment protocol described above with additional administrations of stem cells (from Wharton's Jelly allograft) and exosomes (described below in brain TPE treatment).

iii) Brain TPE treatment: the treatment protocol included the combined TPE treatment protocol described above with some modifications in the oral compositions and additional administrations of naltrexone, neuropeptides, stem cells (from Wharton's Jelly allograft), and exosomes.

5 days prior to the first TPE session and every other TPE session thereafter (3rd TPE and 5th TPE), 5 mg of tetrapeptide epitalon was administered via subcutaneous injection to the patients daily for 10 consecutive days. Before the first TPE session, 750 mcg intranasal spray of adrenocorticotropin (4-10) SEMAX was administered to the patients once a day until the last TPE session was completed.

Immediately after the TPE session was completed, in addition to IV infusion of supplements and glutathione administration, the following were administered to the patients: a) 20 intranasal sprays (about 1 mL) of Wharton's Jelly allograft contained in Vitti Labs WJ-PURE PLUS 1 over 10 minutes; b) 20 intranasal sprays (about 1 mL) of exosomes, growth factors, cytokines, and nucleic acids contained in Vitti Pure EV PURE PLUS 1 over 10 minutes; and c) 3.5 mL intravenous injection of exosomes, growth factors, cytokines, and nucleic acids contained in Vitti Pure EV PURE PLUS 1. This protocol of administration of stem cells and exosomes was also used in the heart TPE treatment and immunity treatment.

After the first TPE was completed, low dose naltrexone was taken orally by the patient at a dose of 1.5 mg once daily for 2 weeks, then the dose was increased to 3 mg taken by the patient once daily for the next 2 weeks, then the dose was increased to 4.5 mg taken by the patient once daily for the next 19 weeks. Additionally, after the first TPE was completed, the patient was administered DMSA orally at 250 mg on Monday through Wednesday every week for 1 to 52 weeks.

The oral composition for brain TPE treatment comprised the components shown in Table 4 plus antioxidants comprising 120 mg of Ginkgo biloba Leaf Extract with 24% Ginkoflavonglycosides/6% Terpene Lactones; herbs comprising 100 mg of Hericium erinaceus extract, 100 mg of Withania somnifera Root Extract, and 100 mg of Bacopa Monnieri Extract; amino acid comprising 100 mg of phosphatidylserine and 100 mg of L-theanine; and other components comprising 30 mg vinpocetine and 100 mg of Panax ginseng 5% Powdered Extract.

iv) Cancer TPE treatment: the treatment protocol included the combined TPE treatment protocol described above with some modifications in the oral compositions, and additional administrations of naltrexone, anti-inflammatory, and anti-fungal medications.

After the first TPE was completed, the patient was administered naltrexone, thymosin al, met enkephalin, methylene blue, nystatin, diflucan, colloidal silver, and mebendazole. The patients took 1.5 mg of naltrexone once daily for two weeks, and then 3 mg of naltrexone once daily for two weeks. Next, the dose was increased to 4.5 mg once daily for 19 weeks. Thymosin al was injected subcutaneously to the patient at a dose of 1.6 mg once daily for 1 to 52 weeks. Met enkephaline was injected subcutaneously to the patient at a dose of 1.5 mg once daily for 1 to 52 weeks. Methylene blue is taken orally by the patient at a dose of 50 mg once daily for 1 to 52 weeks. Nystatin liquid was taken orally by the patient at a dose of 500,000 units four times a day for 1 to 52 weeks. Diflucan was taken orally by the patient at a dose of 100 mg once daily for 1 to 52 weeks. Colloidal silver 2000 ppm was taken orally by the patient at a dose of 10 mL four times a day for 1 to 52 weeks. Mebendazole was taken orally by the patient at a dose of 200 mg once daily for 1 to 52 weeks. Additionally, after the first TPE was completed, the patient was administered DMSA orally at a dose of 250 mg on Monday through Wednesday every week for 1 to 52 weeks.

The oral composition for cancer TPE treatment comprised the components shown in Table 4 plus herbs comprising 1800 mg Agaricus extract, 20 mg of melatonin, 400 mg Allium sativum; and antioxidants comprising 1500 mg sea cucumber, 360 mg of artemisinin, 3 mg Capsicum, 1000 mg of oxaloacetate, 4000 mg of Curcuma longa Extract, 250 mg genistein, 30 mg Asimina triloba, 50 mg lycopene, 1000 mg Theracurmin™ HP, and 1 tablet of Artemax™ Plus containing 20 mg artemether and 120 mg lumefantrine.

v) Immunity TPE treatment: the treatment protocol included the combined TPE treatment protocol described above and additional administrations of naltrexone and regenerative materials. Additionally, after the first TPE was completed, the patient was administered DMSA orally at a dose of 250 mg on Monday through Wednesday every week for 1 to 52 weeks.

The administration of naltrexone followed the same protocol as brain TPE and cancer TPE treatment. The administration of regenerative materials followed the same protocol as described for brain TPE treatment and heart TPE treatment.

### 3. Patient Selection

In all the treatments described herein, stable patients capable of all advanced activities of daily living and having glomerular filtration rate (GFR) greater than 60 mm/min/1.73 were included. Patients that were excluded from the study included: patients with vascular dementia, patients with unstable disease such as angina, patients on medications for ventricular arrhythmia, seizures, or anticoagulation, patients who had ever undergone TPE for any reason, and pregnant patients.

i) Combined TPE and TPE only treatments: thirteen patients, aged 58 to 70, in good overall health with no significant morbidities were recruited. Four of thirteen patients received control TPE (TPE only) treatment and nine of thirteen received combined TPE treatment.

ii) Heart TPE treatment: only patients with non-calcified plaque greater than 100 mm³ and not deemed in eminent threat of cardiovascular event by a cardiologist were qualified to participate. Also, these patients were already maximized on medications (patients likely to have any anti-inflammatory or cholesterol lowering therapy added to their regimen for the following 6-12 months were excluded) and would otherwise need a bypass surgery to treat the atherosclerosis.

iii) Brain TPE treatment: patients with signs of cognitive decline as determined by the CNS-VS assessment or who wished to improve their cognitive functions (but also having a SLUM score>21) were included in the study. Patients must not have vascular dementia. Patients must have MRI without contrast prior to the treatment such as there were no signs of multiple infarcts.

iv) Cancer TPE treatment: patients who were undergoing cancer therapy were excluded.

### 4. Data Collection

For combined TPE and TPE only treatments, two laboratory tests and three laboratory tests were performed before and after the treatment respectively. 192 biomarkers of inflammation, aging, oxidative stress, metabolism, toxins (e.g., heavy metals, environmental toxins, mycotoxins), and diseases (e.g., cancer, osteoarthritis, liver fibrosis) were measured. Only biomarkers that showed abnormal values in the first two laboratory tests before the treatment were included and measured in the 3 laboratory tests after the treatment, except for the following biomarkers: 8-hydroxy-2′-deoxyguanosine (8-OHdG), Klotho, myeloperoxidase (MPO), cluster of differentiation 3 (CD3), CD4, CD8, CD19, CD56, Fibrinogen activity, and Tumor Necrosis Factor α (TNFα). Data from the National Health and Nutrition Examination Survey (NHANES) were utilized to determine the reference level of toxicity.

### 5. Measurements of Toxins and Biomarkers

Tandem liquid chromatography-mass spectrometer (LC-MS), which can detect compounds at the pg/mL level, was used.

Environmental Toxins: LC-MS was used to measure the environmental toxins. The mass spectrum of the sample determined the concentration of each analyte measured. The analyte results were expressed by normalizing to the quantity of creatinine measured to account for urine dilution variations.

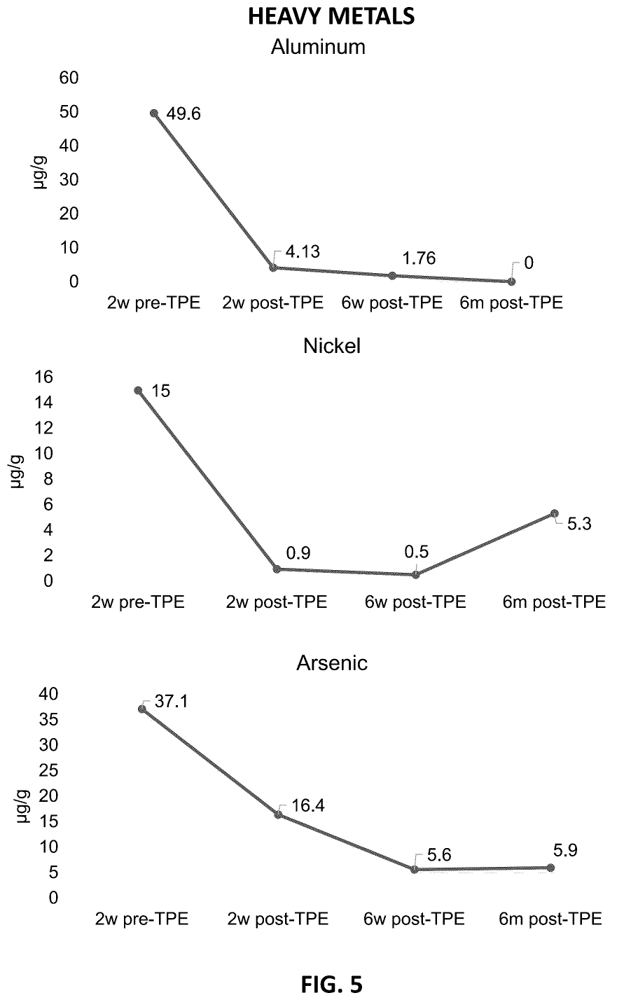

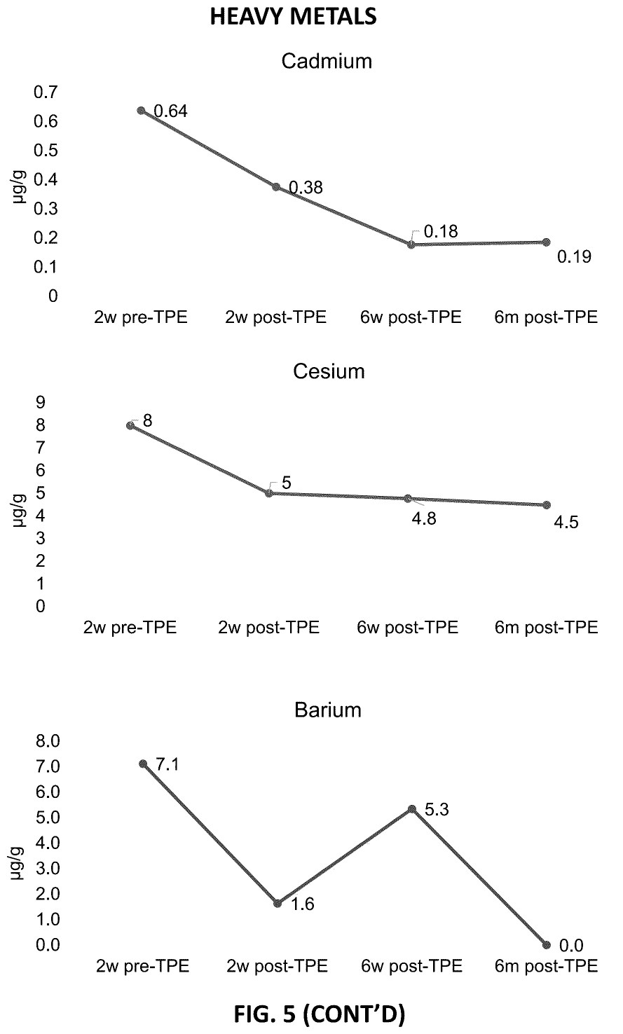

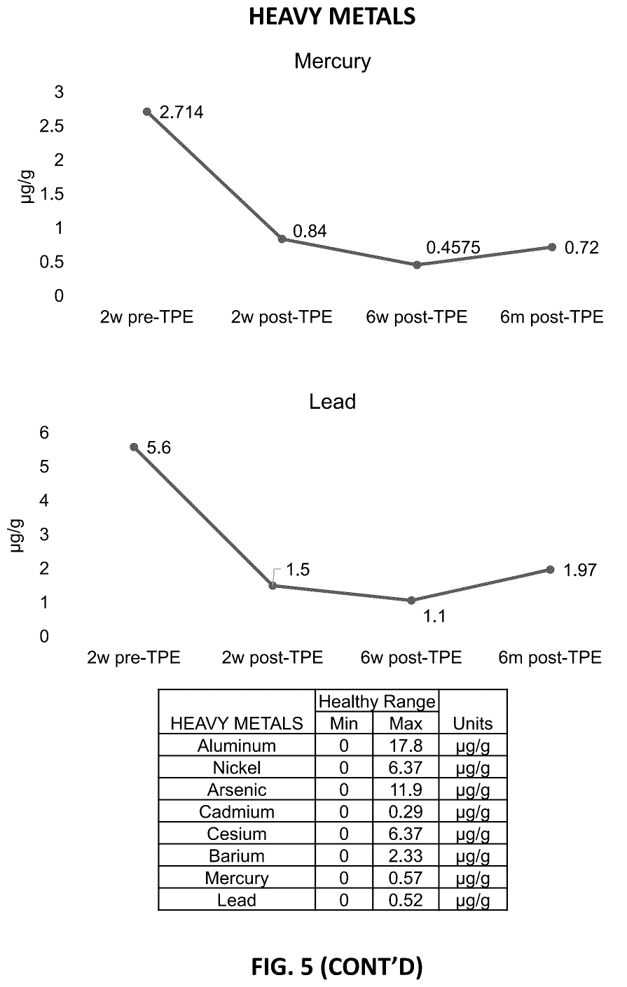

Heavy Metals: the Heavy Metals test used inductively coupled plasma mass spectrometry (ICP-MS). The mass spectrum of the sample determined the concentration of each analyte measured. The analyte results were expressed by normalizing to the quantity of creatinine measured to account for urine dilution variations (FIG. 5).

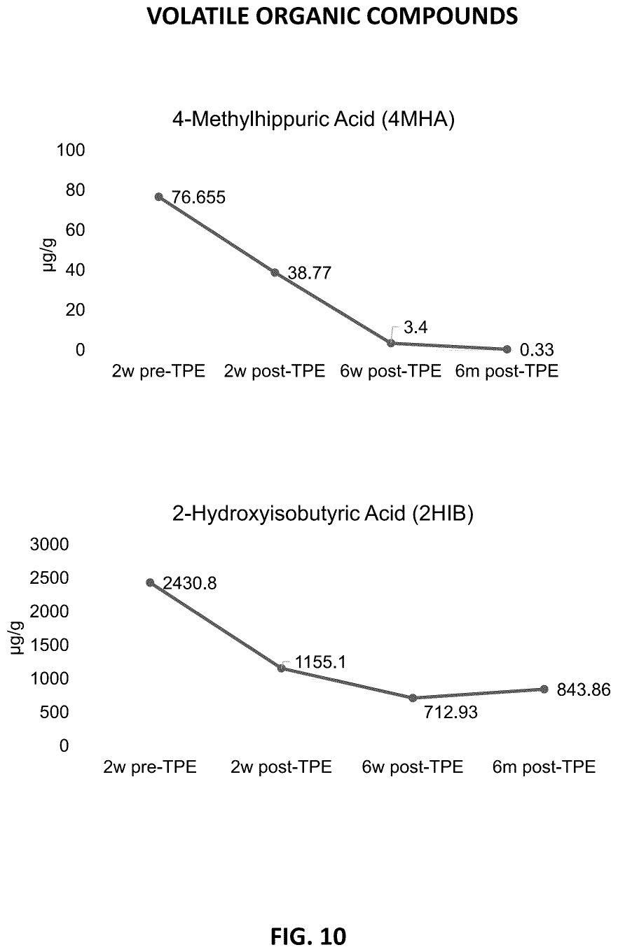

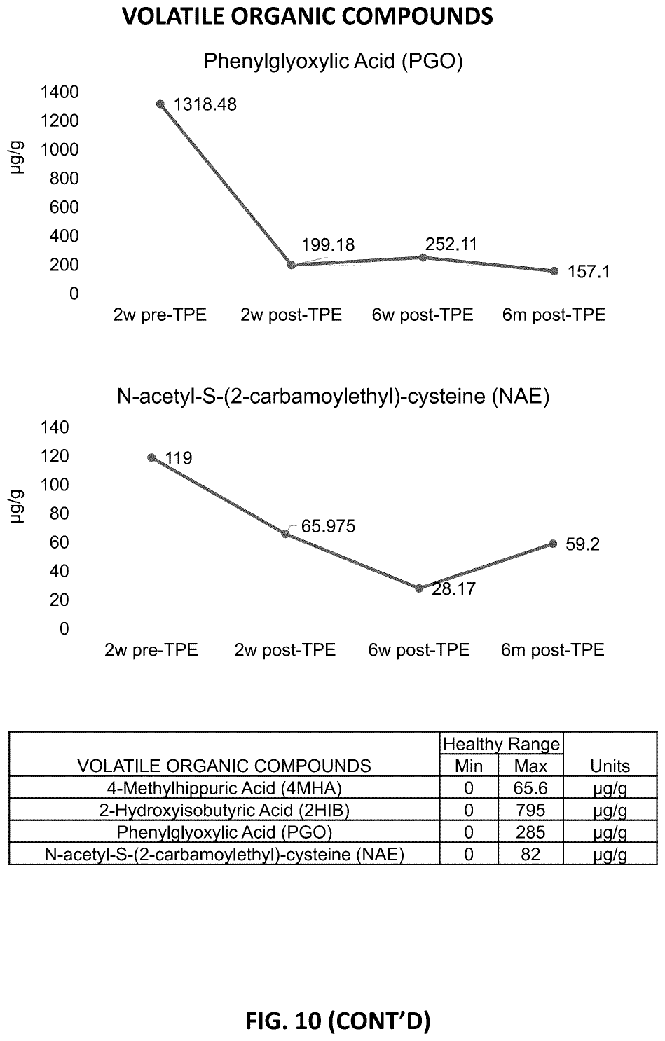

Organic Acids: the Organic Acids test used gas chromatography with tandem mass spectrometry (GC-MS/MS). The analyte results were expressed by normalizing to the quantity of creatinine measured to account for urine dilution variations (FIG. 10).

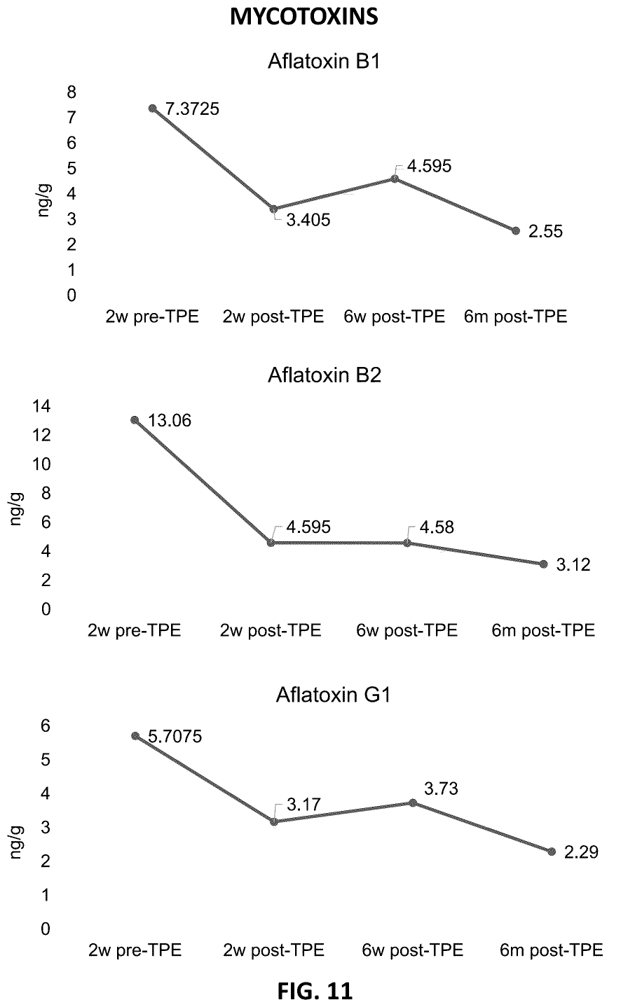

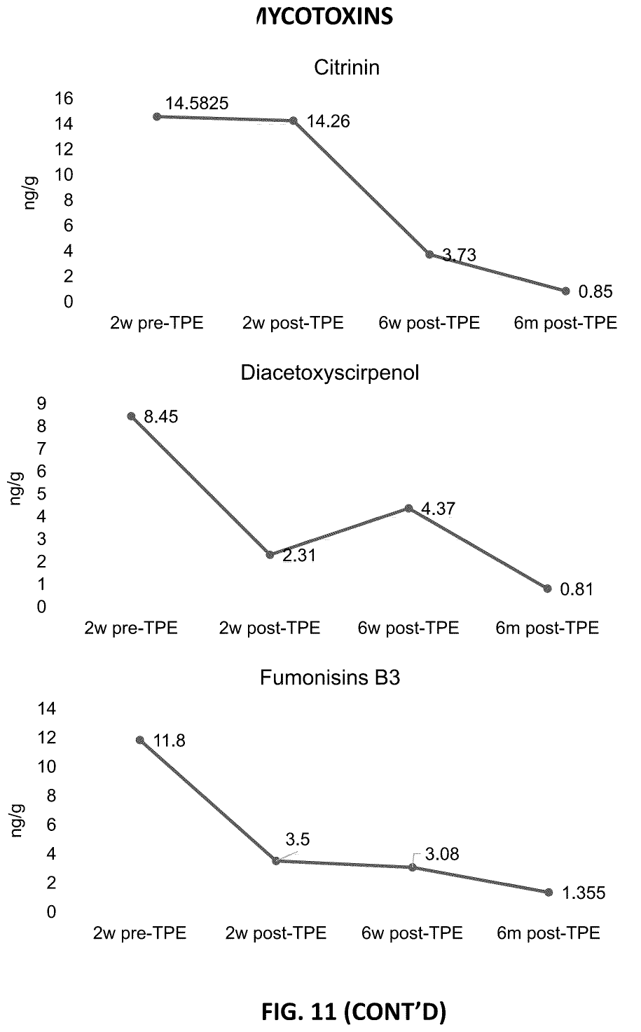

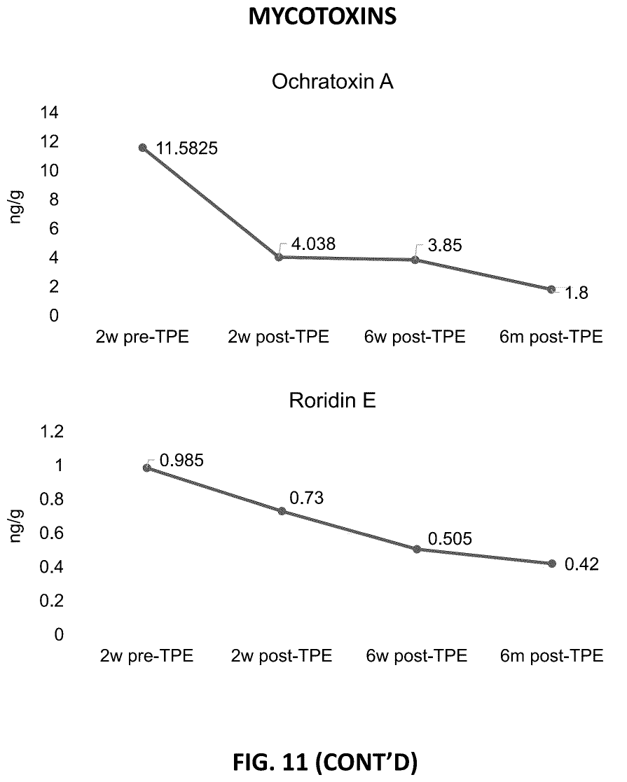

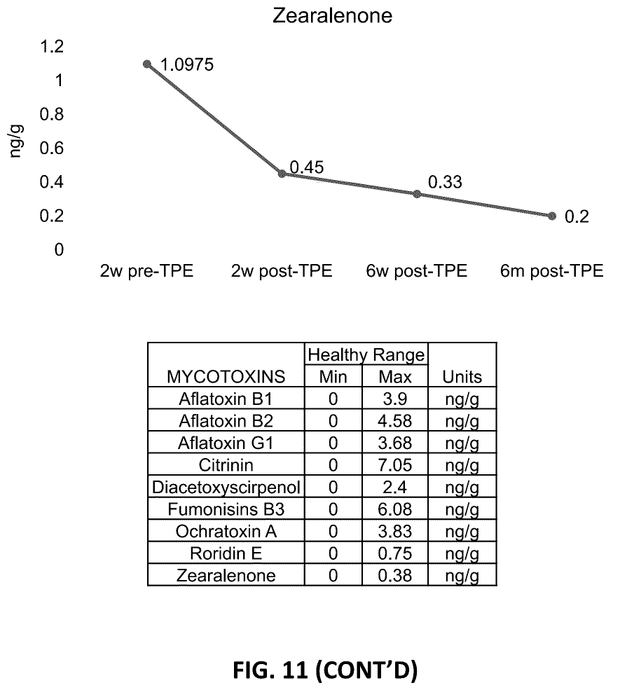

Mycotoxins and PFAS: the Mycotoxins and PFAS test used liquid chromatography and mass spectrometry (LC-MS/MS). The mass spectrum of the sample determined the concentration of each analyte measured. The analyte results were expressed by normalizing to the quantity of creatinine measured to account for urine dilution variations (FIG. 11 and FIG. 12).

Microplastics: blood samples were collected in patients one week and two weeks before and 2 weeks and 6 months after receiving the combined TPE treatment, to measure microplastics and microplastic polymers. The analysis included quantification and characterization of microplastics size range between 3 and 500 μm with Fourier Transform Infrared Spectroscopy (FTIR). Sample preparation was based on the protocol detailed in the Van Velzen et al., Environ. Int. 163: 107199 (2022)) (FIG. 13).

Biological markers: blood and/or urine samples were sent to LabCorp for appropriate laboratory tests to measure the level of biological markers. Information regarding the specific test for each marker can be found from www.labcorp.com/test-menu/search. The laboratory tests included flow cytometry, ELISA, enzymatic assay, latex immunoturbidimetry, and others.

### 6. Other Laboratory and Functional Tests

Cognitive Function: cognitive function was assessed using the method previously described in Lee et al., J. Restorative Medicine 14(1): 1-12 (2024). Briefly, the cognitive function was assessed by a computerized neurocognitive test, CNS-VS, with CNS software (CNS Vital Signs, Chapel Hill, NC). The CNS-VS assesses nine cognitive functions: memory, verbal memory, visual memory, processing speed, executive functioning, psychomotor speed, reaction time, complex attention, and cognitive flexibility. The Neurocognition Index (NCI) is calculated based on five tasks: memory, psychomotor speed, reaction time, complex attention, and cognitive flexibility. The computer report from the CNS-VS test provides subject scores, standard scores, percentiles, and assessments according to a 5-degree scale for each of the nine cognitive functions examined and the Neurocognition Index. These assessments are as follows: above average (more than 109), average (90-109), low average (80-89), low (70-79), and very low (less than 70) (Gualtieri et al., Arch. Clin. Neuropsychol. 21(7): 623-43 (2006)).

Telomere and biological age: these were evaluated using TruAge (developed by TruDiagnostic Inc., Lexington, KY). Peripheral whole blood samples were obtained using the lancet and capillary method, then mixed with lysis buffer to preserve the cells. DNA extraction was performed, and 500 ng of DNA was subjected to bisulfite conversion using the EZ DNA Methylation Kit from Zymo Research, following the manufacturer's protocol. The bisulfite-converted DNA samples were randomly allocated to designated Illumina Infinium EPIC850k Beadchip wells. The samples were amplified, hybridized onto the array, and subsequently stained. After washing steps, the variety was imaged using the Illumina iScan SQ instrument to capture raw image intensities, enabling further analysis. The algorithms analyzed by TruAge include first- (Horvath and Hannum), second- (phenoAge, systemsAge, OMICAge, and GrimAge), and third-generation clocks (DunedinPACE). This study predicted telomere length via DNA methylation data using DNAmTL (Lu et al., Aging 11(16): 5895-923 (2019)).

Cancer Biomarkers and Circulating Tumor Cells: a patient's blood sample was collected and sent to RGCC laboratory for analysis. Briefly, PBMC were isolated from the blood samples and gene expression analysis was performed based on Apostolou et al., Oncotarget 10(36): 3328-38 (2019), on more than 90 genes. The expression profile was used for the classification of sample as cancerous or healthy. Biomarkers indicative of early stages of cancer genesis were measured in the peripheral blood sample such as CD19, CD31, CD34, CD44, CD99, CD133, epithelial cell adhesion molecule (EpCam), and pan-cytokeratin (panCK).

### 7. Sources of Neuropeptides, Mesenchymal Stem Cells (MSCs), and Exosomes

Epitalon and SEMAX: these peptides were obtained from an FDA-approved 503A compounding pharmacy in the United States. Both peptides were kept in the refrigerator.

Mesenchymal Stem Cells (MSCs) and Exosomes: Vitti Labs WJ-PURE PLUS 1 was used to provide Mesenchymal Stem Cells and Vitti Pure EV PURE PLUS 1 was used to provide exosomes. Vitti Labs is an FDA-registered, American Association of Tissue Banks (AATB) accredited, Current Good Manufacturing Practice (CGMP) certified tissue bank. The donated tissue was bio-ethically bestowed and collected from the hospital operating room from a full-term c-section birth with a healthy mother and baby. All tissue procured was screened by using Chagas enzyme immunoassay (EIA) test antibodies to the human immunodeficiency virus, type 1 and type 2 (anti-HIV-1 and anti-HIV-2) Plus O, Nucleic Acid Test (NAT) for HIV-1, hepatitis B surface antigen (HbsAg), hepatitis B, core total Ab, hepatitis C virus Ab (anti-HCV), cytomegalovirus (CMV) total Ab, HTLV I/II Ab, West Nile virus (WNV), ZIKA NAT, Nucleic Acid Test (NAT) for the hepatitis B virus, hepatitis C virus, and human immunodeficiency virus (HBV/HCV), syphilis, and Lyme disease. In addition, the donor was screened following the AATB standards for medical history, genetic screening, and vaccinations, including COVID-19. Once an AATB-accredited procurement agency collected the tissue, sterility was maintained throughout the custody of the tissue.

MSCs and exosomes were isolated from the umbilical cord tissue of Wharton's jelly. They were tested using Flow Cytometry Services from RayBiotech (Corners, GA) for the following surface markers: CD20−, CD34−, CD45−, HLA-DR−, DC73+, CD90+, CD105+, CD166+. The MSCs were then expressed to release exosomes with CD9+, CD63+, and CD81+ surface markers. The exosomes were collected and purified using Tangential Flow Filtration. The number of exosomes consisted of 15 billion per milliliter. The size distribution was between 50 and 90 nm in diameter. These measurements were taken using direct Stochastic Optical Reconstruction Microscopy via nanoimager from Oxford Nanoimaging. The concentrations were measured using Tunable Resistive Pulse Sensing (TRPS) from Izon Sciences. Quality assurance was taken for sterility by testing 20% of the final lot, using USP 71 guidelines for sterility testing (U.S. Pharmacopeia, “General chapters: sterility tests,” ftp.uspbpep.com/v29240/usp29nf24s0_c71.html, last accessed: Sep. 18, 2024), USP 85 endotoxin testing (U.S. Pharmacopeia, “General chapters: bacterial endotoxin,” www.uspbpep.com/usp29/v29240/usp29nf24s0_c85.html, last accessed: Sep. 18, 2024), and USP 1116 for environmental testing (U.S. Pharmacopeia, “General chapters: microbiological evaluation of clean rooms and other controlled environments,” ftp.uspbpep.com/v29240/usp29nf24s0_c1116.html, last accessed: Sep. 18, 2024) following all cGMP standards and ISO 5 conditions. They were shipped in a validated shipper on dry ice and stored in a cryo-freezer at negative 80° C. at the Institute for Hormonal Balance.

### 6. IV and Oral Compositions

The IV and oral compositions were designed and customized to replenish lost nutrients from TPE and supplement essential molecules such as anti-inflammatory molecules, antioxidants, amino acids, vitamins, and minerals. To formulate the IV and oral compositions, several biological processes that are considered to be the most critical for healthy functions were first selected, including the mitochondrial metabolic pathway, anti-inflammatory pathway, antioxidant pathway, detoxifying pathway, and essential metabolic and enzymatic pathways. The components were then selected according to their functions and associations with these processes. Based on the assumption that the selected biological processes are compromised due to long-term toxicity, the dose of each component was determined so that it would be sufficient to improve the biological functions but safe enough. Table 3 and Table 4 below show the components in each composition.

TABLE 3
IV compositions

| | | | | | | | |
|---|---|---|---|---|---|---|---|
| | | Conc. | Vol. | | Dose | mOsm | |
| Component | Conc. | Units | (mL) | Dose | Units | /mL | mOsm |

Minerals

| | | | | | | | |
|---|---|---|---|---|---|---|---|
| Calcium gluconate 10% | 100 | mg/mL | 10 | 1000 | mg | 2.04 | 20 |
| Magnesium chloride | 300 | mg/mL | 7 | 2100 | mg | 2.95 | 20 |
| Molybdenum | 250 | mcg/mL | 1 | 250 | mcg | 0.34 | 0 |
| Potassium chloride | 2 | meq/mL | 3 | 6 | meq | 0.001 | 0 |
| Selenium | 200 | mcg/mL | 2 | 400 | mcg | 0.1 | 0 |
| Zinc sulfate | 10 | mg/mL | 0.5 | 5 | mg | 0 | 0 |

Vitamins

| | | | | | | | |
|---|---|---|---|---|---|---|---|
| Ascorbic acid | 500 | mg/mL | 30 | 15000 | mg | 5.94 | 178 |
| B-complex 100¹ | 1 | | 3 | 3 | | 2.04 | 6 |
| Dexpanthenol | 250 | mg/mL | 6 | 1500 | mg | 1.22 | 7 |
| Hydroxocobalamin | 2 | mg/mL | 3 | 6 | mg | 0.31 | 0 |
| Pyridoxine | 100 | mg/mL | 1 | 100 | mg | 1.46 | 1 |

Amino Acids

| | | | | | | | |
|---|---|---|---|---|---|---|---|
| L-carnitine | 500 | mg/mL | 2 | 1000 | mg | 2.3 | 4 |
| Taurine | 50 | mg/mL | 8 | 400 | mg | 1.5 | 12 |
| Sterile water | | | 250 | | | 0 | 0 |
| Total | | | 326 | | | | 248 |
| Glutathione² | 200 | mg/mL | 5 | 1000 | mg | 0 | 0 |

¹One unit of B-complex 100 comprising 100 mg/mL thiamine, 2 mg/mL riboflavin, 100 mg/mL niacin, 2 mg/mL dexpanthenol, 2 mg/mL pyridoxine.
²Given over 2 minutes after infusion complete.

TABLE 4
Oral composition in one scoop (about 60 g)

| | | |
|---|---|---|
| Component | Dose | Units |

Minerals

| | | |
|---|---|---|
| Calcium D-Glucarate | 500 | mg |
| Chromium Polynicotinate | 25 | mcg |
| Copper Gluconate | 1 | mg |
| Magnesium Bisglycinate Chelate Buffered | 100 | mg |
| Manganese Glycinate | 5 | mg |
| Molybdenum Glycinate Chelate | 50 | mcg |
| Potassium Iodide | 25 | mcg |
| Zinc Bisglycinate Chelate | 5 | mg |

Vitamins

| | | |
|---|---|---|
| Ascorbic Acid (vitamin C) | 500 | mg |
| Biotin | 50 | mcg |
| Cholecalciferol (vitamin D3) | 2500 | IU |
| L-5-Methyltetrahydrofolate, glucosamine salt | 1000 | mcg |
| Methylcobalamin | 500 | mcg |
| Niacin (vitamin B3) | 15 | mg |
| Nicotinamide Riboside Chloride (NR) (vitamin B3) | 750 | mg |
| Pantothenic Acid | 100 | mg |
| Pyridoxine | 15 | mg |
| Riboflavin | 15 | mg |
| Thiamine | 1 | mg |
| Vitamin A Palmitate | 1000 | mcg |
| Vitamin E as Mixed Tocopherols | 50 | IU |
| Nicotinamide Mononucleotide | 1000 | mg |

Amino Acids

| | | |
|---|---|---|
| L-Alanine | 175 | mg |
| L-Arginine HCl | 100 | mg |
| L-Glutamine | 1250 | mg |
| L-Glycine | 300 | mg |
| L-Lysine HCl | 500 | mg |
| L-Methionine | 70 | mg |
| L-Ornithine HCl | 50 | mg |
| L-Proline | 250 | mg |
| L-Tyrosine | 50 | mg |
| Taurine | 100 | mg |
| Trimethyl Glycine (TMG) - Betaine Anhydrous | 1000 | mg |

Anti-inflammatory agents

| | | |
|---|---|---|
| Bromelain 2400 gelatin dissolving units (GDU) | 250 | mg |
| Noni Fruit Extract 4:1 | 50 | mg |
| Omega-3 comprising 2:1 eicosapentaenoic acid | 200 | mg |
| (EPA):docosahexaenoic acid (DHA) | | |
| Pterostilbene | 50 | mg |
| Resveratrol Root Extract | 100 | mg |
| Skullcap Root Extract (30% Baicalin) | 250 | mg |
| Superoxide dismutase 14000 IU/g | 75 | mg |
| Unicaria Tomentosa Bark Extract (3% Oxindole | 250 | mg |
| Alkaloids) | | |

Antioxidants

| | | |
|---|---|---|
| Alpha Lipoic Acid | 100 | mg |
| Artichoke Flower Extract (5% Polyphenols) | 100 | mg |
| Astaxanthin | 5 | mg |
| Boswellia Serrata extract | 1000 | mg |
| Broccoli Seed Extract (13% Glucoraphanin) | 75 | mg |
| Coenzyme Q10 blend from Sunflower Lecithin | 50 | mg |
| Dandelion Root Extract | 250 | mg |
| Grape Seed Extract | 100 | mg |
| Green tea (Camellia Sinensis) Leaf Extract with 50% | 100 | mg |
| Epigallocatechin gallate (EGCG) | | |
| Hops Extract | 125 | mg |
| L-Glutathione reduced | 100 | mg |
| Micro PQQ | 10 | mg |
| Milk Thistle Seed extract (80% Silymarin) | 100 | mg |
| N-Acetyl-L-Cysteine (NAC) | 100 | mg |
| Punica granatum Fruit Extract (30% Punicalagins) | 250 | mg |
| Quercetin | 500 | mg |
| Rosemary Extract | 100 | mg |
| Schisandra Berry Extract | 100 | mg |
| Shilajit Extract | 100 | mg |
| Tumeric (Curcuma Longa) Extract | 250 | mg |

Saccharides

| | | |
|---|---|---|
| D-Ribose | 1000 | mg |
| Larch Arabinogalactan Powder | 500 | mg |
| 1,3 Beta Glucan 85% | 500 | mg |

Herbs

| | | |
|---|---|---|
| Bitter Melon Extract | 100 | mg |
| Ginger Rhizome Extract (5% Gingerols) | 200 | mg |

Protein

| | | |
|---|---|---|
| Rice Protein | 15000 | mg |

### Example 2: The Combined TPE Treatment Protocol was More Effective than the Control TPE Treatment in Increasing the Level of Biomarkers of Immune System and Longevity

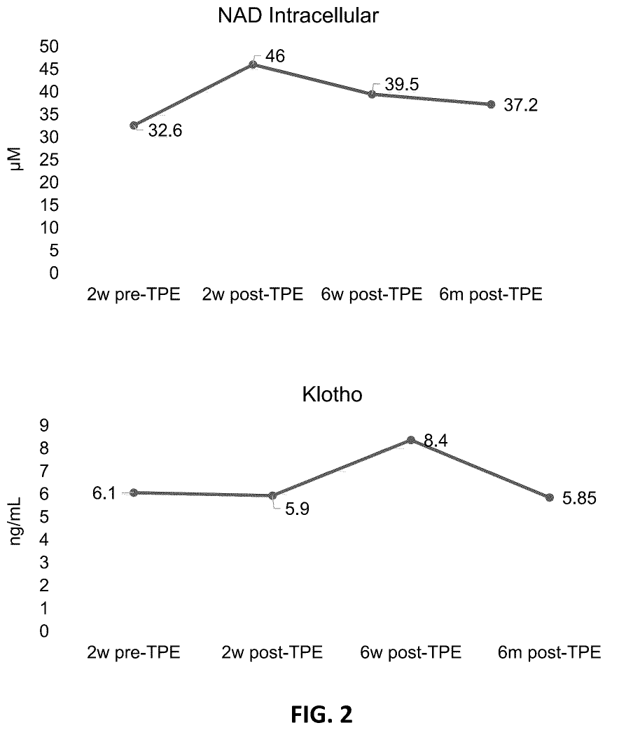

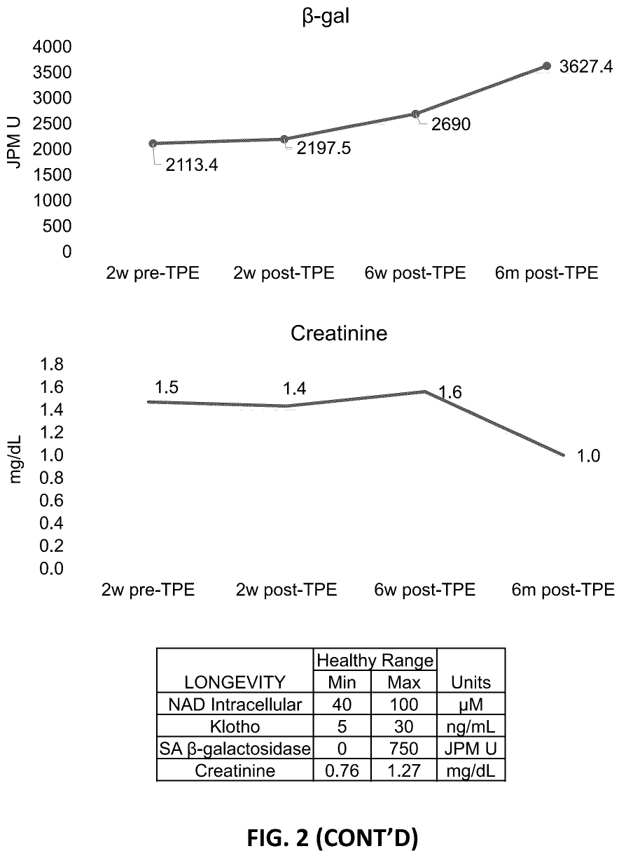

The immune system is important in determining overall well-being and has been linked to longevity. Particularly, the aging immune system loses the ability to protect against infections and cancer, as well as to support appropriate wound healing (Weyand et al., Ann. Am. Thorac. Soc. 13(Suppl5): S422-28 (2016)). Biomarkers of the immune system, such as CD4, CD19, CD8, and CD56, showed a significant increase at 2 weeks (day 14), 6 weeks (day 42), and up to 6 months (day 180) of between 12% and 47% on average after the combined TPE treatment as compared to before the treatment (FIG. 1 and Table 5). Similarly, biomarkers of longevity such as Klotho and nicotinamide adenine dinucleotide intracellular (NAD intracellular) also showed an increase of up to 40% on average after of the combined TPE treatment (FIG. 2 and Table 5). Klotho is an anti-aging single-pass membrane protein predominantly produced in the kidney, with shedding of the amino-terminal extracellular domain into the systemic circulation. Circulating levels of soluble Klotho decrease with age, and the klotho gene is associated with increased risk of age-related diseases (Cheikhi et al., J. Gerontol. A Biol. Sci. Med. Sci. 74(7): 1031-42 (2019); Correa et al., Sci. Rep. 12(1): 17587 (2022)). NAD intracellular plays an essential role in cellular oxidation-reduction (redox) reactions and is responsible for accepting high-energy electrons and carrying them to the electron transport chain (ETC) to synthesize adenosine triphosphate (ATP). Age-dependent decline in NAD levels contributes to aging, namely through DNA damage, epigenetic alteration, deregulated nutrient-sensing, loss of proteostasis, altered cellular communication, cellular senescence, stem cell exhaustion, mitochondrial dysfunction, compromised autophagy, and possibly telomere attrition (Kincaid et al., Exp. Biol. Med. (Maywood) 245(17): 1594-614 (2020)).

In contrast, the control TPE treatment also showed a modest increase of average 17% of the immune system biomarkers and 30% average of the longevity biomarkers (Table 5). The increase for combined TPE treatment was at least 150% and 40% more than the TPE only treatment in the immune system and longevity biomarkers respectively (Table 6). These results suggest that the combined TPE treatment was more effective than the TPE treatment to replenish these biomarkers.

### Example 3: The Combined TPE Treatment Protocol was More Effective than the Control TPE Treatment in Decreasing the Level of Biomarkers of Inflammation, Oxidative Stress, and Senescence

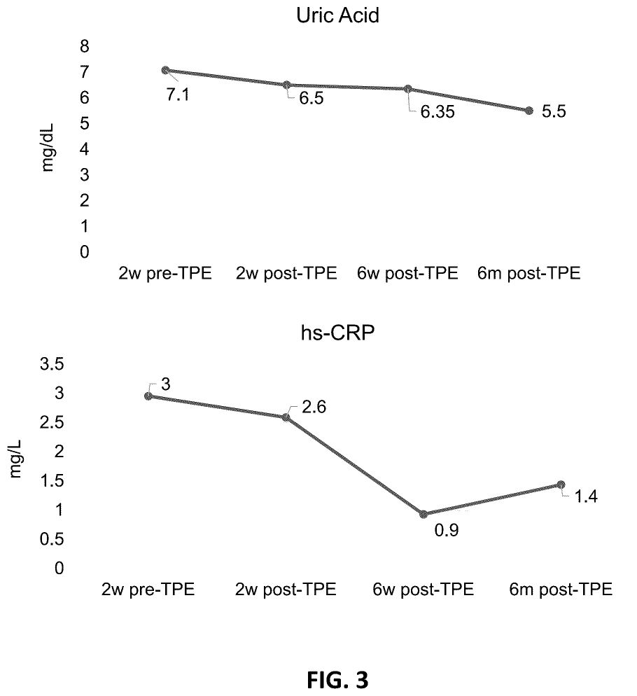

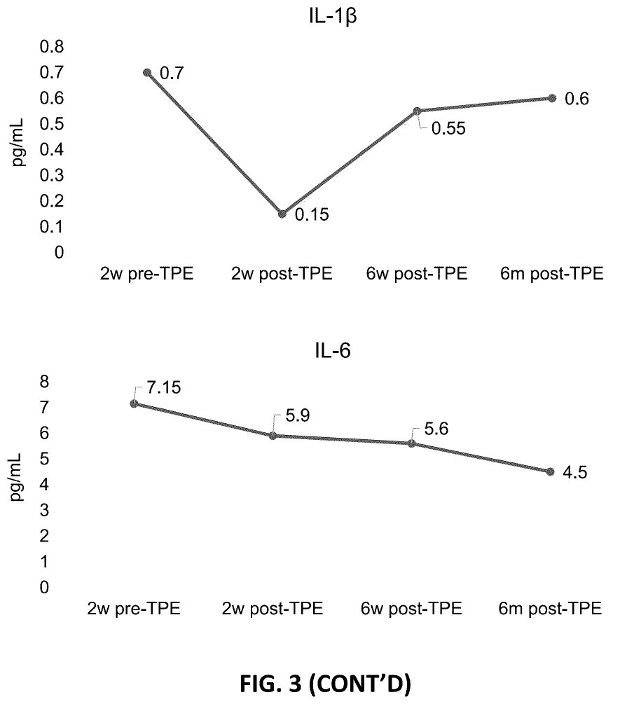

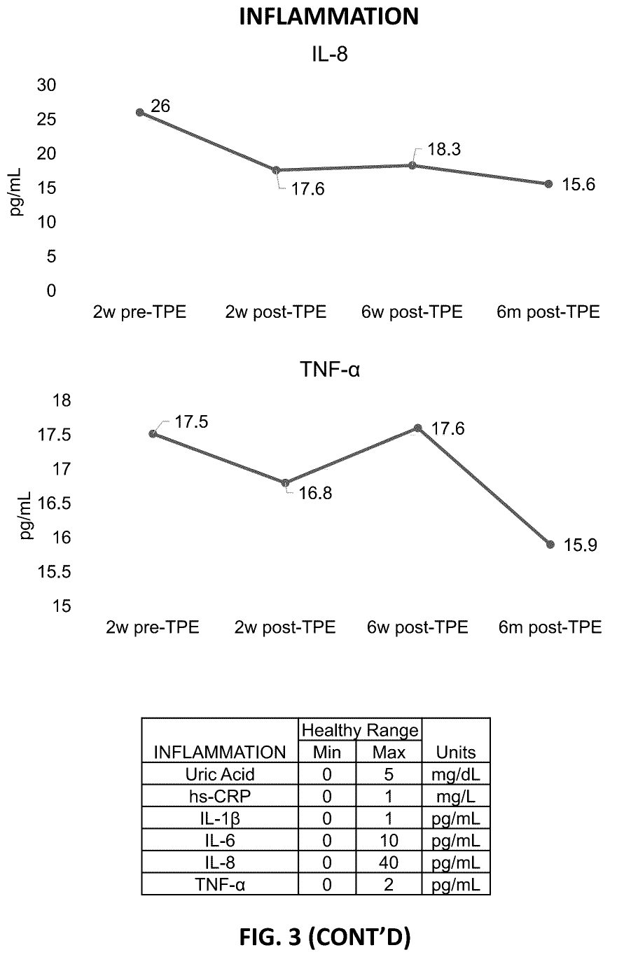

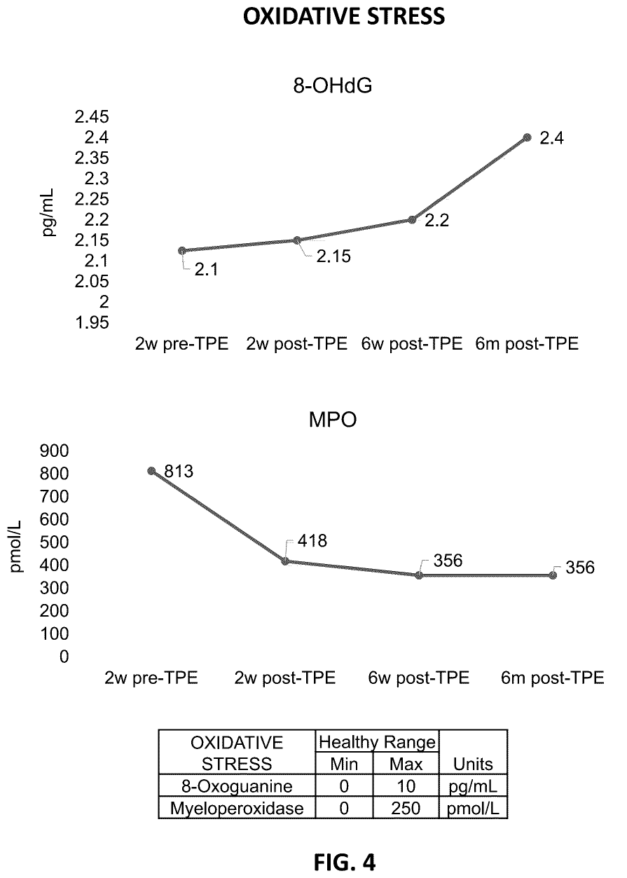

It has been shown that aging is associated with systemic chronic inflammation and cellular senescence (Li et al., Signal Transduct. Target Ther. 8(1): 239 (2023)). One of the causes of chronic inflammation can be attributed to oxidative stress (Hussain et al., Oxid. Med. Cell Longev. 2016:2016: 7432797 (2016)). Thus, the biomarkers of inflammation and oxidative stress were measured and assessed. Combined TPE treatment reduced most of these biomarkers from the abnormal level before treatment to a healthy and normal level (lighter shade) after treatment (FIG. 3, FIG. 4, and Table 5). In circumstances where the biomarkers were not reduced to a normal level, the overall decreasing trend indicated that combined TPE treatment was effective at suppressing these biomarkers. Additionally, when compared with the control TPE treatment, combined TPE treatment was at least 100% and 60% more effective in reducing inflammation and oxidative stress respectively (Table 5 and Table 6).

### Example 4: Combined TPE Treatment Protocol was Surprisingly Effective in Removing Toxins

It is known in the art that elimination of toxins, especially heavy metals, from the body is challenging with no known effective method (Rajkumar et al., “Heavy Metal Toxicity,” in StatPearls [Internet](Treasure Island (FL), StatPearls Publishing, 2024); Sears, Scientific World Journal 2013: 219840 (2013)). Traditionally, TPE is performed on patients to remove pathologic targets in the plasma which were causing the clinical symptoms (Mahmoud et al., Neurocrit. Care. 34(1): 301-11 (2021)). However, it is uncommon to perform TPE on healthy patients and no studies have reported on TPE and its capacity to remove other toxins. The results of this study show that combined TPE treatment and TPE only treatment significantly reduced a range of toxins from heavy metals, herbicides, pesticides, phenols, phthalates, volatile organic compounds, and mycotoxins (FIG. 6 through FIG. 12, and Table 5). Surprisingly, the methods of detoxification disclosed utilizing combined TPE treatment comprising TPE and administration of IV and oral compositions were found to be more robust in clearing toxins than the control TPE/TPE only treatment (Table 5), namely at least 40% more effective on average in clearing cadmium, barium, tungsten, mercury, thallium, BPA, 4-Nonylphenol, DEDTP, Glyphosate, MEHP, Diacetoxyscirpenol, Fumonisins B3, and Ochratoxin A than TPE only treatment (Table 6). This study suggests that the results obtained with combined TPE treatment were not solely due to TPE but a combination of TPE, IV infusion, and administration of oral supplements.

Interestingly, the trend of change in the tested biomarkers continued even after 6 months following completion of both combined TPE treatment and TPE treatment (Table 5). During this period, patients in the combined TPE treatment continued taking the oral supplements/composition and all patients donated plasma at a blood bank every 6 to 12 weeks. The data indicate that the combined TPE treatment was effective in removing toxins and boosting beneficial biological processes, and that the results were sustained for a long period of time with daily administration of oral supplements and regular plasma donation.

TABLE 5
Percentage change (% change) at 2 weeks (2 w), 6 weeks (6 w), and 6 months
(6 m) after combined TPE treatment and control TPE treatment (TPE only) compared
to before the treatment. In combined TPE protocol, patients continued taking
the oral composition. Some patients in both groups donated plasma every 6-12
weeks and continued lifestyle changes to avoid exposure toxins.

| Biomarkers | COMBINED TPE | | | | TPE ONLY | | | |
|---|---|---|---|---|---|---|---|---|
| and Toxins | 2 w | 6 w | 6 m | Average | 2 w | 6 w | 6 m | Average |

Immune System

| | | | | | | | | |
|---|---|---|---|---|---|---|---|---|
| CD3 | 31.02 | 15.02 | 76.68 | 40.91 | 13.37 | 36.51 | −0.36 | 16.51 |
| CD4 | 30.78 | 15.54 | 71.72 | 39.35 | 3.24 | 25.69 | 2.70 | 10.54 |
| CD8 | 18.69 | 14.09 | 39.62 | 24.13 | 3.47 | 28.82 | −18.40 | 4.63 |
| CD19 | 11.87 | 14.06 | 8.31 | 11.41 | −3.52 | 6.68 | 6.72 | 3.29 |
| CD56 | 56.62 | 47.43 | 36.69 | 46.91 | −0.42 | −2.76 | 1.53 | −0.55 |

Inflammation

| | | | | | | | | |
|---|---|---|---|---|---|---|---|---|
| Uric Acid | −9.06 | −11.42 | −13.39 | −11.29 | −10.73 | −12.79 | 6.78 | −5.58 |
| (UA) | | | | | | | | |
| hs-C Reactive | −12.29 | −63.43 | −71.39 | −49.04 | −5.98 | −15.36 | −22.31 | −14.55 |
| Protein | | | | | | | | |
| Interleukin-1β | −74.24 | −13.64 | −45.45 | −44.44 | −16.28 | −11.92 | −15.98 | −14.73 |
| Interleukin-6 | −11.26 | −29.53 | −58.12 | −32.97 | −2.96 | −0.33 | −3.18 | −2.16 |
| Interleukin-8 | −30.57 | −30.12 | −32.45 | −31.05 | −59.52 | −51.20 | −50.81 | −53.84 |
| Tumor | −5.91 | −2.15 | 0.31 | −2.58 | −23.64 | −12.73 | 19.27 | −5.70 |
| Necrosis | | | | | | | | |
| Factor alpha | | | | | | | | |

Longevity

| | | | | | | | | |
|---|---|---|---|---|---|---|---|---|
| Klotho | −0.08 | 23.54 | 6.73 | 10.06 | 0.23 | 19.23 | 0.79 | 6.75 |
| Protein | | | | | | | | |
| NAD | 39.96 | 26.86 | 8.56 | 25.13 | 27.89 | −0.63 | 20.95 | 16.07 |
| Intracellular | | | | | | | | |
| β- | 6.47 | 32.73 | 80.20 | 39.80 | 33.57 | −19.22 | 71.85 | 28.73 |
| galactosidase | | | | | | | | |
| Creatinine | −2.03 | −0.53 | −13.04 | −5.20 | 7.84 | 9.54 | 11.26 | 9.55 |

Kidney function

| | | | | | |
|---|---|---|---|---|---|
| Estimated | −2.03 | −5.53 | −13.04 | 9.54 | 9.54 |
| glomerular | | | | | |
| filtration rate | | | | | |
| (eGFR) | | | | | |

Oxidative Stress

| | | | | | | | | |
|---|---|---|---|---|---|---|---|---|
| 8-Oxoguanine | −18.59 | −46.28 | | | | | | |
| Myeloperoxidase | −45.81 | −26.29 | −26.29 | −32.80 | −98.37 | −34.02 | −98.16 | −76.85 |
| 8-Hydroxy-2′- | 6.97 | 11.82 | 28.18 | 15.66 | 85.11 | −0.78 | | 42.17 |
| deoxyguanosine | | | | | | | | |
| (8-OHdG) | | | | | | | | |

Heavy Metals

| | | | | | | | | |
|---|---|---|---|---|---|---|---|---|
| Aluminum | −92.33 | −93.19 | −100.00 | −95.17 | | | | |
| Nickel | −90.63 | −95.88 | −47.26 | −77.92 | −86.08 | −57.19 | | −71.64 |
| Arsenic | −51.66 | −78.69 | −82.06 | −70.80 | −89.69 | | −89.63 | −89.66 |
| Cadmium | −49.01 | −74.37 | −72.89 | −65.42 | 65.29 | 19.01 | −39.47 | 14.94 |
| Antimony | −78.57 | −100.00 | −100.00 | −92.86 | −75.00 | | −75.00 | −75.00 |
| Tellurium | −92.79 | −67.57 | −54.95 | −71.77 | −50.00 | −77.56 | −73.02 | −66.8 |
| Cesium | −38.19 | −43.47 | −50.50 | −44.05 | | | | |
| Barium | −75.66 | −11.95 | −100.00 | −62.54 | 3.91 | −56.68 | −46.17 | −32.98 |
| Gadolinium | −100.00 | | | −100.00 | −100.00 | −100.00 | −71.43 | −90.48 |
| Tungsten | −92.21 | −95.92 | −100.00 | −96.04 | −81.25 | −80.00 | −28.00 | −63.08 |
| Mercury | −61.93 | −79.85 | −67.86 | −69.88 | −35.40 | −60.25 | −30.43 | −42.03 |
| Lead | −58.45 | −83.02 | −25.58 | −55.68 | −56.47 | −9.45 | −71.66 | −45.86 |
| Thallium | −55.52 | −90.24 | −63.03 | −69.60 | 39.24 | 8.86 | | 24.05 |
| Beryllium | −63.29 | −31.65 | −49.37 | −48.10 | −13.58 | −100.00 | | −56.79 |
| Tin | −74.37 | −100.00 | −68.45 | −80.94 | | −60.25 | | −60.25 |
| Bismuth | −68.34 | −82.12 | −77.08 | −75.85 | | −9.45 | | −9.45 |
| Thorium | −100.00 | | −100.00 | −100.00 | | −8.86 | | −8.86 |
| Uranium | −100.00 | −81.82 | −100.00 | −93.94 | | | | |

Environmental Toxins

| | | | | |
|---|---|---|---|---|
| Perchlorate | −67.65 | −58.85 | −74.75 | −67.08 |

Volatile Organic Compounds

| | | | | | | | | |
|---|---|---|---|---|---|---|---|---|
| N-acetyl-S- | −44.51 | −76.70 | −51.70 | −57.64 | −63.73 | −28.21 | −90.78 | −60.91 |
| (2- | | | | | | | | |
| carbamoylethyl)- | | | | | | | | |
| cysteine (NAE) | | | | | | | | |
| 2- | | | | | −27.79 | −23.74 | −54.35 | −35.29 |
| Hydroxyethyl | | | | | | | | |
| Mercapturic | | | | | | | | |
| Acid (HEMA) | | | | | | | | |
| N-Acetyl | | | | | −52.58 | −91.95 | | −72.27 |
| Propyl | | | | | | | | |
| Cysteine | | | | | | | | |
| (NAPR) | | | | | | | | |
| Tiglylglycine | −63.64 | −27.27 | −36.36 | −42.42 | −18.52 | −33.33 | −48.15 | −33.33 |
| (TG) | | | | | | | | |
| mono-(2- | −60.88 | −76.98 | −67.79 | −68.55 | | | | |
| ethyl-5- | | | | | | | | |
| oxohexyl) | | | | | | | | |
| phthalate | | | | | | | | |
| (MEOHP) | | | | | | | | |
| 4- | −49.42 | −95.56 | −99.57 | −81.52 | | | | |
| Methylhippuric | | | | | | | | |
| Acid (4MHA) | | | | | | | | |
| 2- | −57.98 | −71.86 | −73.67 | −67.84 | | | | |
| Hydroxyisobutyric | | | | | | | | |
| Acid (2HIB) | | | | | | | | |
| Phenylglyoxylic | −84.89 | −80.88 | −88.08 | −84.62 | | | | |
| Acid (PGO) | | | | | | | | |
| mono-(2- | −60.88 | −76.98 | −67.79 | −68.55 | | | | |
| ethyl-5- | | | | | | | | |
| oxohexyl) | | | | | | | | |
| phthalate | | | | | | | | |
| (MEOHP) | | | | | | | | |

Herbicides

| | | | | | | | | |
|---|---|---|---|---|---|---|---|---|
| Atrazine | −76.47 | −88.24 | −76.47 | −80.39 | −83.33 | | −83.33 | −83.33 |
| Glyphosate | −62.26 | −67.96 | −83.58 | −71.27 | −43.97 | −49.29 | | −46.63 |
| 2,4- | −44.29 | −76.69 | −63.58 | −61.52 | | | | |
| Dichlorophen | | | | | | | | |
| oxyacetic | | | | | | | | |
| Acid (2,4-D) | | | | | | | | |

Paraben

| | | | |
|---|---|---|---|
| Butylparaben | −18.80 | −32.14 | −25.47 |

Pesticides

| | | | | | | | | |
|---|---|---|---|---|---|---|---|---|
| Diethyl- | −46.34 | −51.22 | −26.83 | −41.46 | −26.53 | −26.53 | | −26.53 |
| dithiophosphate | | | | | | | | |
| (DEDTP) | | | | | | | | |
| Dimethyl- | −12.44 | −43.66 | −52.24 | −36.11 | | | | |
| dithiophosphate | | | | | | | | |
| (DMDTP) | | | | | | | | |
| Dimethyl- | −33.47 | −75.34 | −28.58 | −45.80 | −55.39 | −49.94 | −67.73 | −57.69 |
| phosphate (DMP) | | | | | | | | |
| Dimethyl- | −36.59 | −39.82 | −58.90 | −45.10 | | | | |
| thiophosphate | | | | | | | | |
| (DMTP) | | | | | | | | |
| 1,12- | −72.44 | | −80.63 | −76.54 | | | | |
| Dodecanedioic | | | | | | | | |
| acid (DDA) | | | | | | | | |

Phenols

| | | | | | | | | |
|---|---|---|---|---|---|---|---|---|
| Bisphenol A | −53.74 | −66.28 | −60.23 | −60.08 | 6.98 | −4.02 | | 1.48 |
| (BPA) | | | | | | | | |
| Triclosan | −21.60 | −45.44 | −36.81 | −34.62 | | | | |
| 4- | −62.75 | −45.10 | | −53.93 | −18.13 | | −53.64 | −35.89 |
| Nonylphenol | | | | | | | | |

Phthalates

| | | | | | | | |
|---|---|---|---|---|---|---|---|
| mono-2- | −99.97 | −94.68 | −97.33 | −35.77 | −41.23 | | −38.50 |
| ethylhexyl | | | | | | | |
| phthalate | | | | | | | |
| (MEHP) | | | | | | | |
| mono-(2- | | | | −53.08 | −81.27 | −97.01 | −77.12 |
| ethyl-5- | | | | | | | |
| hydroxyhexyl) | | | | | | | |
| phthalate | | | | | | | |
| (MEHHP) | | | | | | | |

Mycotoxins

| | | | | | | | | |
|---|---|---|---|---|---|---|---|---|
| Aflatoxin B1 | −63.64 | −27.27 | −36.36 | −42.42 | | | | |
| Aflatoxin B2 | −40.21 | −42.26 | −55.22 | −45.90 | | | | |
| Aflatoxin G1 | −64.82 | −64.93 | −76.11 | −68.62 | | | | |
| Aflatoxin G2 | −58.05 | | −99.33 | −78.69 | | | | |
| Aflatoxin M1 | | −65.31 | | −65.31 | | | | |
| Citrinin | −0.66 | −74.81 | −94.08 | −56.52 | −33.29 | −57.20 | | −45.25 |
| Deoxynivalenol | | −10.19 | | −10.19 | | | | |
| Diacetoxyscirpenol | −70.12 | −52.34 | −89.52 | −70.66 | −27.44 | −30.61 | | −29.03 |
| Enniatin B1 | −60.29 | −79.41 | −73.53 | −71.08 | | | | |
| Fumonisins | −31.50 | −45.22 | −57.93 | −44.88 | −89.32 | −94.42 | | −91.87 |
| B1 | | | | | | | | |
| Fumonisins | −49.52 | −79.34 | −80.71 | −69.86 | −32.88 | −66.21 | | −49.55 |
| B3 | | | | | | | | |
| Gliotoxin | | −33.87 | | −33.87 | | | | |
| Ochratoxin A | −64.91 | −70.66 | −85.72 | −73.76 | −34.55 | −56.65 | −35.00 | −42.07 |
| Roridin E | −19.78 | −51.19 | −53.85 | −41.61 | | | | |
| Zearalenone | −53.36 | −77.32 | −78.95 | −69.88 | | | | |

Brain Autoimmune Markers

| | | | | | | | | |
|---|---|---|---|---|---|---|---|---|
| Anti- | −26.84 | −26.84 | −46.94 | −33.54 | −36.76 | −45.10 | −54.25 | −45.37 |
| Cerebellum | | | | | | | | |
| (IgG + IgA) | | | | | | | | |
| Anti-HSV1 | 6.75 | −23.6 | 15.78 | −0.38 | −17.65 | −13.73 | 17.65 | −4.58 |
| (IgG + IgA) | | | | | | | | |
| Anti-Neuron | | −15.38 | | −15.38 | | −9.09 | | −9.09 |
| specific | | | | | | | | |
| enolase (IgG + | | | | | | | | |
| IgA) | | | | | | | | |
| Anti-Neuron | −28.57 | −28.57 | | −28.57 | | −0.5 | | −0.5 |
| specific | | | | | | | | |
| enolase IgM | | | | | | | | |
| Anti-Purkinje | −6.02 | −15.07% | −13.04 | −11.38 | −9.09 | −9.09 | −9.09 | −9.09 |
| cell (IgG + | | | | | | | | |
| IgA) | | | | | | | | |
| Anti-Purkinje | −20.00 | −33.33 | −46.67 | −33.33 | | | | |
| cell IgM | | | | | | | | |
| Anti-Tubulin | | −7.69 | | −7.69 | | | | |
| (IgG + IgA) | | | | | | | | |

TABLE 6
Percentage difference (A) in average percent
change of TPE only (without supplementation)
vs. combined TPE (with supplementation).

| | |
|---|---|
| | Percentage A in average |
| | % change of TPE only |
| | compared to combined |
| Biomarkers and Toxins | TPE (%) |

Immune System

| | |
|---|---|
| CD3 | 147.8 |
| CD4 | 273.2 |
| CD8 | 421.2 |
| CD19 | 246.6 |
| CD56 | 8629.7 |

Inflammation

| | |
|---|---|
| Uric Acid (UA) | 102.3 |
| hs-C Reactive Protein | 237.0 |
| Interleukin-1ß | 201.8 |
| Interleukin-6 | 1428.7 |

Longevity

| | |
|---|---|
| Klotho Protein | 49.1 |
| NAD Intracellular | 56.4 |
| β-galactosidase | 38.5 |
| Creatinine | 154.5 |

Oxidative Stress

| | |
|---|---|
| 8-Hydroxy-2′-deoxyguanosine (8-OhdG) | 62.9 |

Heavy Metals

| | |
|---|---|
| Nickel | 8.8 |
| Cadmium | 537.8 |
| Antimony | 23.8 |
| Tellurium | 7.3 |
| Barium | 89.6 |
| Gadolinium | 10.5 |
| Tungsten | 52.2 |
| Mercury | 66.3 |
| Lead | 21.4 |
| Thallium | 389.4 |

Environmental Toxin

| | |
|---|---|
| Tiglylglycine (TG) | 27.3 |

Herbicides

| | |
|---|---|
| Glyphosate | 52.8 |

Pesticides

| | |
|---|---|
| Diethyldithiophosphate (DEDTP) | 56.3 |

Phenols

| | |
|---|---|
| Bisphenol A (BPA) | 4159.7 |
| 4-Nonylphenol | 50.3 |

Phthalate

| | |
|---|---|
| mono-2-ethylhexyl phthalate (MEHP) | 152.8 |

Mycotoxins

| | |
|---|---|
| Citrinin | 24.9 |
| Diacetoxyscirpenol | 143.4 |
| Fumonisins B3 | 41.0 |
| Ochratoxin A | 75.3 |

Brain autoimmune marker

| | |
|---|---|
| Anti-Purkinje cell (IgG + IgA) | 25.2 |

### Example 5: The Combined TPE Treatment Protocol was Effective in Removing Per- and Polyfluorinated Substances (PFAS)

PFAS are a group of chemicals used to make fluoropolymer coatings and products that resist heat, oil, stains, grease, and water. Studies have shown that some exposure to low environmental levels of PFAS may affect reproduction, thyroid function, immune system, growth and development, and injure the liver. Scientists at the Centers for Disease Control and Prevention (CDC) have measured at least 12 PFAS in blood serum (CDC, “Per- and Polyfluorinated Substances (PFAS) Factsheet,” <https://www.cdc.gov/biomonitoring/PFAS_FactSheet.html> last accessed Mar. 3, 2024). Currently, no medical treatments are available to reduce PFAS in the body (Agency for Toxic Substances and Disease Registry, “PFAS information for clinicians,” <https://www.atsdr.cdc.gov/pfas/resouorces/pfas-information-for-clinicians.html#print> last accessed Mar. 3, 2024). The level of PFAS in urine was measured before and after combined TPE treatment. Seven PFAS were measured including: hexafluoropropylene oxide dimer acid (HPFO-DA), Perfluoro-1-[1,2,3,4-13C4]octanesulfonic acid, Perfluoro-1-heptane sulfonic acid (PFHpS), Perfluorododecanoic acid (PFDoA), Perfluorohexane Sulfonic Acid (PFHxS), Perfluorooctane sulfonic acid (PFOS), Perfluorooctanoic acid (PFOA), Perfluoropentanoic acid (PFPeA). The level of all measured PFAS were reduced significantly after the combined TPE treatment (FIG. 12 and Table 7).

TABLE 7
Percent change of PFAS in urine sample collected from patients 6 weeks after
receiving combined TPE treatment vs. before receiving the treatment.

| | Before | After | Percentage |
|---|---|---|---|
| | treatment | treatment | change |
| Test | (μg/g) | (μg/g) | (%) |
| GenX/HPFO-DA | 2.82 | 2.014 | −28.58 |
| Perfluoro-1-heptane sulfonic acid (PFHpS) | 0.719 | 0.533 | −25.87 |
| Perfluorododecanoic acid (PFDoA) | 0.696 | 0.396 | −43.10 |
| Perfluorohexane Sulfonic Acid (PFHxS) | 0.156 | 0.117 | −25.00 |
| Perfluorooctane sulfonic acid (PFOS) | 0.982 | 0.64 | −34.83 |
| Perfluorooctanoic acid (PFOA) | 0.931 | 0.924 | −0.75 |
| Perfluoropentanoic acid (PFPeA) | 0.467 | 0.299 | −35.97 |

### Example 6: The Combined TPE Treatment Protocol was Effective in Removing Microplastics

Multiple studies have reported the negative impact of microplastics exposure on health outcomes (Wang et al., EBioMedicine 103: 105118 (2024); Lee et al., Yonsei Med J. 64(5): 301-08 (2023)); however, there is currently no effective method to remove microplastics from the body.

The results in FIG. 13 and Table 8 show that the disclosed combined TPE treatment was effective in removing microplastics up to the longest tested time point at 6 months after the treatment. The measured microplastics included polymers such as: polyethylene, poplypropylene, polyamide, polyethylene terephthalate, polytetrafluoroethylene, PMMA, PAN, PEEK, PLA, POM, POB, polyisoprene, PU, PC, PEBA, silicone/polysiloxanes, poly(p-hydroxybutyrate), and other bio-derived and biodegradable plastics.

TABLE 8
Microplastic particles per mL (MP/mL) and percent change of microplastics
in blood sample collected from patients at 2 weeks and 6 months after
receiving combined TPE treatment vs. before receiving the treatment.

| | Pre- | | | | |
|---|---|---|---|---|---|
| | treatment | 2 w | 6 m | 2 w (% | 6 m (% |
| Tested Compound | (MP/mL) | (MP/mL) | (MP/mL) | change) | change) |
| Total Microplastics | 16.2 | 11.7 | 6.67 | −33.52% | −61.49% |

### Example 7: The Brain TPE Treatment Protocol was Effective in Improving Cognitive Function and Increasing Telomere Length

Boada et al., Alzheimers Dement. 16(10): 1412-25 (2020), reported that plasma exchange slowed cognitive and functional decline in patients with mild-to-moderate Alzheimer's disease (AD). Our study showed that TPE, peptides, and human umbilical cord tissue MSCs and exosomes reduced biological age, increased telomere length, and improved cognitive function Lee et al., J. Restorative Medicine 14(1): 1-12 (2024). A brain TPE treatment incorporating the method in Lee et al. and the combined TPE treatment were carried out in patients to assess the biological age, telomere length, and cognitive function. The Lee et al. method included administration of neuropeptide, exosomes, and stem cells to improve body regeneration. After the brain TPE treatment, cognitive function as measured by CNS-VS was improved in the tested patients (Table 9). Additionally, telomere length was increased, and biological age was reduced after the brain TPE treatment. The level of inflammation and oxidative stress biomarkers were reduced and remain low after the treatment. The biomarkers indicative of improved immune system and wellness were increased and sustained after the treatment. All the toxins measured including PFAS, microplastics, heavy metals, and other described above were also reduced after the treatment.

TABLE 9
Percentage change (% change) at 6 weeks (6 w) after brain TPE treatment
and control TPE treatment (TPE only) compared to before the treatment.

| | Combined TPE | | Control TPE | |
|---|---|---|---|---|
| | Percentage | | Percentage | |
| | difference | | difference | |
| | in average | | in average | |
| | before and | | before and | |
| | after | | after | |
| | treatment | Count | treatment | Count |
| Neurocognition Index (NCI) Std Score | 15.15 | 2 | | |
| Composite Memory Std Score | 7.35 | 3 | 6.90 | 1 |
| Verbal Memory Std Score | 16.11 | 2 | | |
| Visual Memory Std Score | 11.54 | 1 | 3.75 | 1 |
| Psychomotor Speed Std Score | 16.09 | 1 | | |
| Reaction Time Std Score | 6.22 | 2 | 0.00 | 1 |
| Complex Attention Std Score | 25.98 | 2 | | |
| Cognitive Flexibility Std Score | 27.19 | 2 | | |
| Executive Function Std Score | 26.03 | 2 | | |
| Simple Attention Std Score | 13.41 | 1 | 0.00 | 1 |

### Example 8: The Heart TPE Treatment is Effective in Halting Progression of Atherosclerosis

It is reported that chronic exposure to low levels of heavy metals increases the risk of cardiovascular diseases (American Heart Association News, “Higher cardiovascular risk linked to toxic metals found in everyday life,” www.heart.org/en/news/2023/06/12/higher-cardiovascular-risk-linked-to-toxic-metals-found-in-everyday-life, published Jun. 12, 2023, last accessed Sep. 18, 2024). Furthermore, oxidative stress and inflammation have been linked to atherosclerosis (Batty et al., Cells 11(23): 2843 (2022)). Thus, a heart TPE treatment comprises the combined TPE treatment and administration of regenerative materials such as stem cells and exosomes, is tested on patients with atherosclerosis who have been maximized on medications and would otherwise need a bypass surgery. It is expected the size of the plaque is significantly reduced after the treatment. The cardiometabolic markers such as those associated with inflammation and oxidative stress biomarkers are reduced and remain low after the treatment. The level of toxins also are reduced after the treatment. The biomarkers indicative of improved immune system and wellness are increased and sustained after the treatment. All the toxins measured including PFAS, microplastics, heavy metals, and other described above are also reduced after the treatment.

### Example 9: The Combined TPE Treatment was Effective in Preventing Early Cancer Development

Accumulated toxins and heavy metals have been reported to underlie many diseases such as cancer (Briffa et al., Heliyon 6(9):e04691, (2020); National Cancer Institute, “Cancer-Causing Substances in the Environment,”<https://www.cancer.gov/about-cancer/causes-prevention/risk/substances> last accessed Mar. 8, 2024). A cancer TPE treatment comprised the combined TPE treatment and administrations of anti-inflammatory and anti-fungal medications was tested on 4 patients with early cancer markers in the blood. In these patients, CD31, CD133, and pan-cytokeratin (panCK) were found to be positive before the treatment. CD31, also known as platelet endothelial cell adhesion molecule-1 (PECAM-1), is a marker of endothelial cells that can be expressed on tumor cells and is involved in cancer. CD133 is a glycoprotein that serves as a marker for cancer stem cells in many types of cancer. Measurement of PanCK was used to confirm the presence of epithelial-origin tumors; detect and categorize tumors; distinguish tumors based on whether or not they originate from the epithelium; diagnose if a tumor has a lot of the HER2 receptor protein on the surface of the cancer cells. The treatment lowered the levels of CD31 and panCK in PBMC to negative detection (Table 10). The levels of CD31, CD133, and panCK remained low for at least 6 months after the treatment was completed. In one patient that was diagnosed with prostate cancer, the cancer TPE treatment reduced the size of inflammation by 90%, the size of a localized cancer in the prostate by 50%, and the size of the prostate by 50%.

TABLE 10
Percentage change (% change) at 6 weeks (6 w) after
cancer TPE treatment and control TPE treatment
(TPE only) compared to before the treatment.

| | Percentage | |
|---|---|---|
| | difference | |
| | in average | |
| | before and | |
| | after | |
| | treatment | Count |
| CD31 | −100.00 | 3 |
| panCK | −100.00 | 3 |

### Example 10: The Immunity TPE Treatment is Effective in Boosting the Immune Health

As shown in Example 2, the combined TPE treatment increased expression of biomarkers indicative of improved immune health. To increase the effectiveness and further promote body healing and regeneration, the immunity TPE treatment comprises administration of stem cells and exosomes in addition to the combined TPE treatment. After the immunity TPE treatment, the level of biomarkers is shifted towards improvement of the immune system lymphocytes as well as improvement of the inflammation.

### Example 11: All TPE Treatments with Supplementations were Effective in Clearing Toxin Levels and Improving Biomarkers Towards Healthy Range

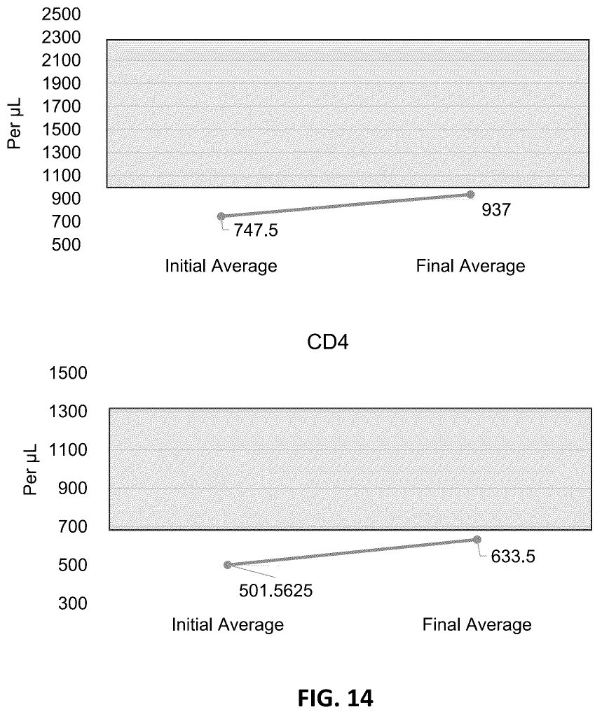

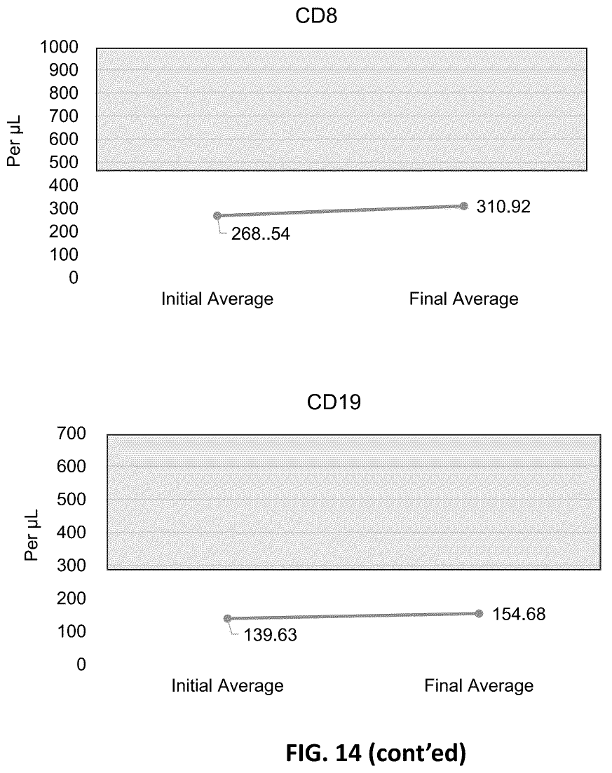

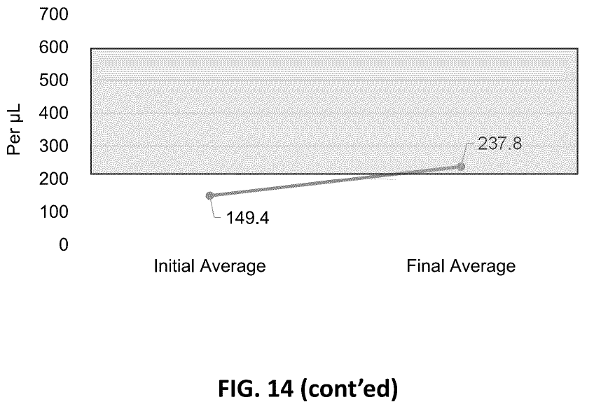

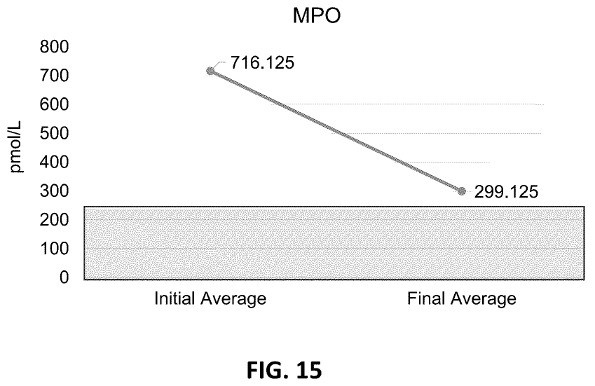

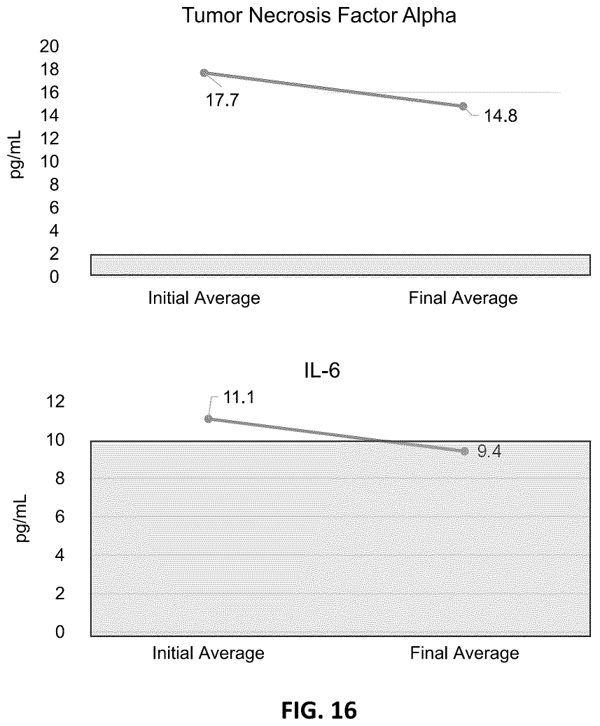

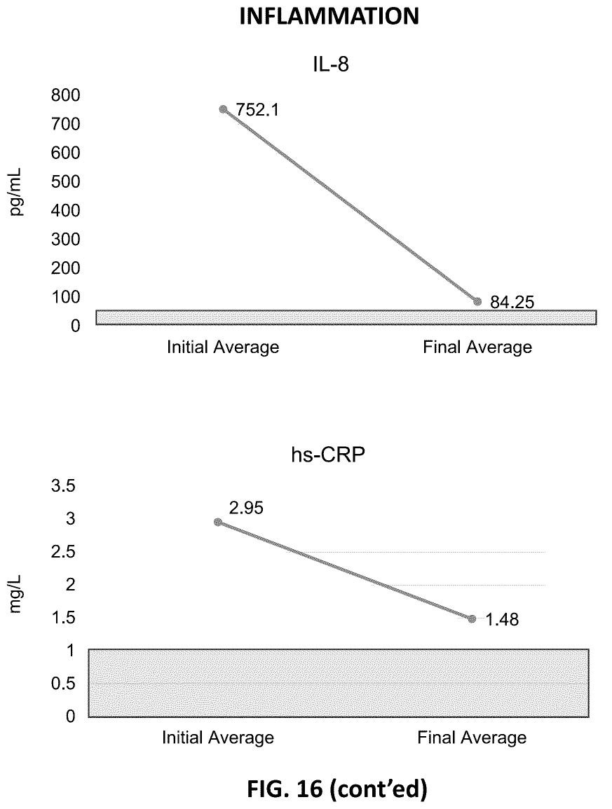

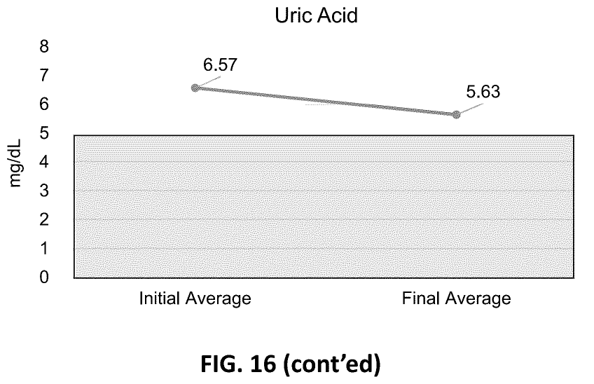

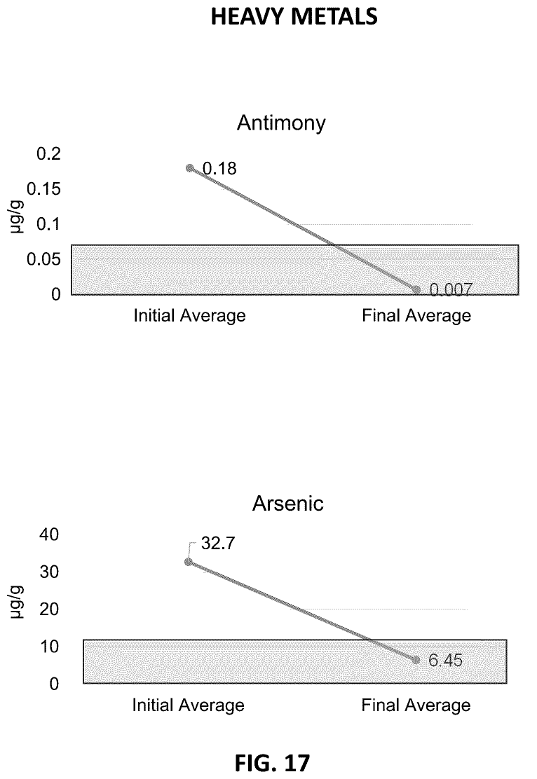

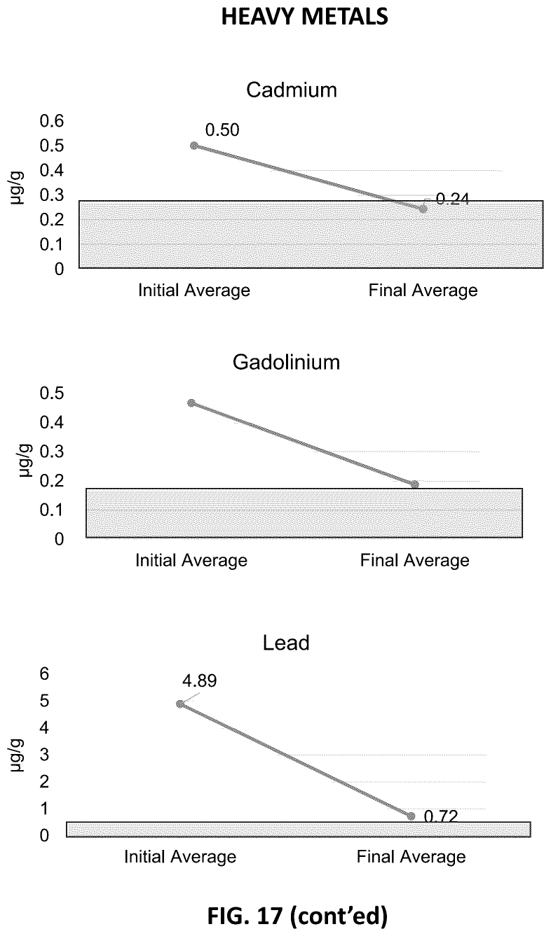

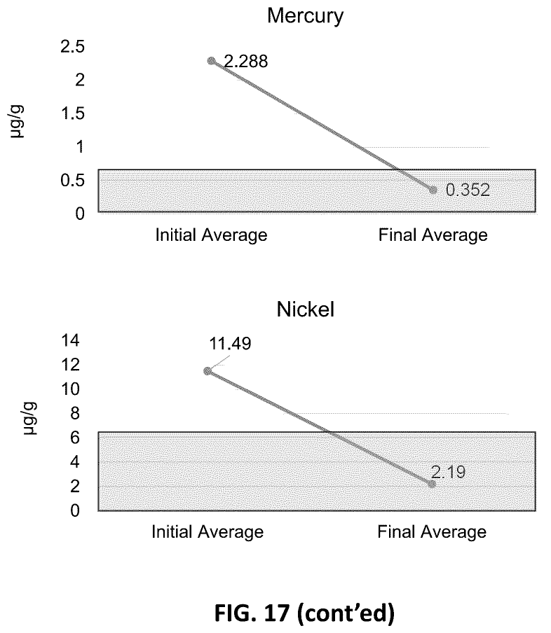

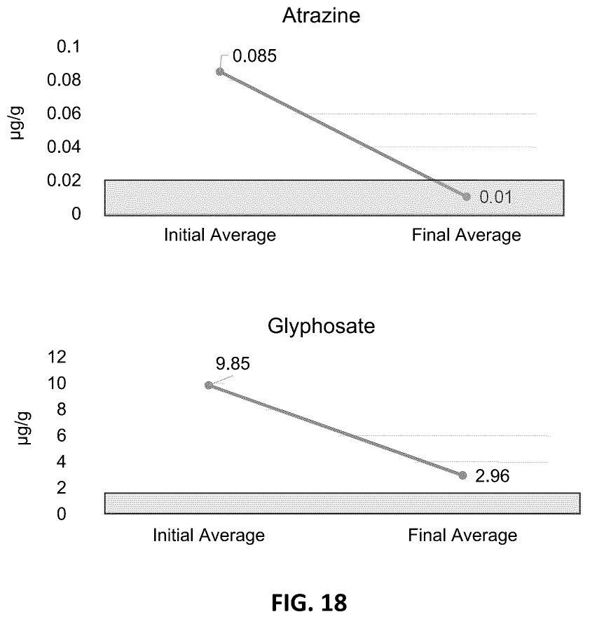

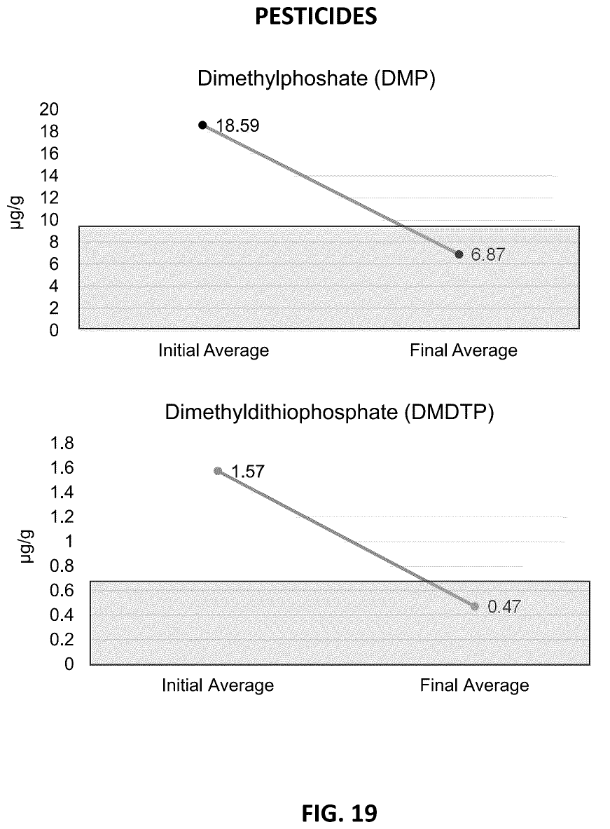

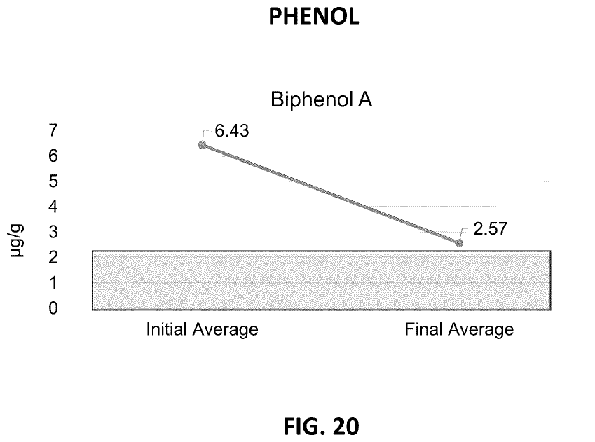

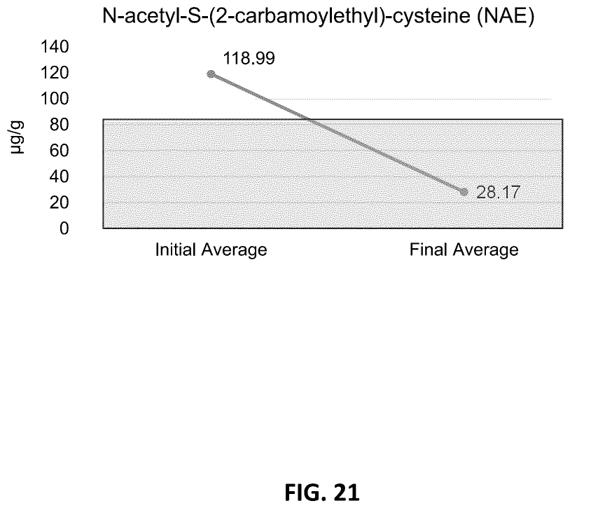

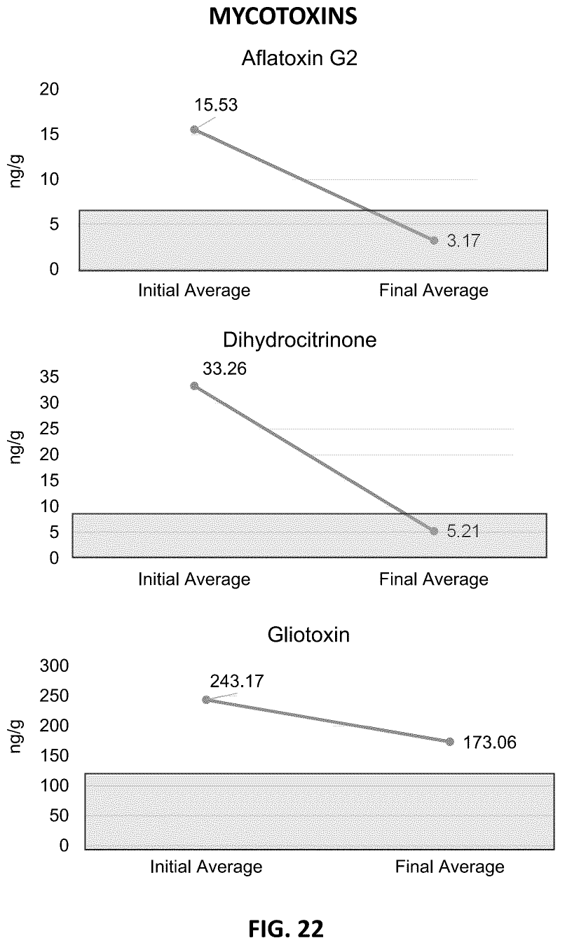

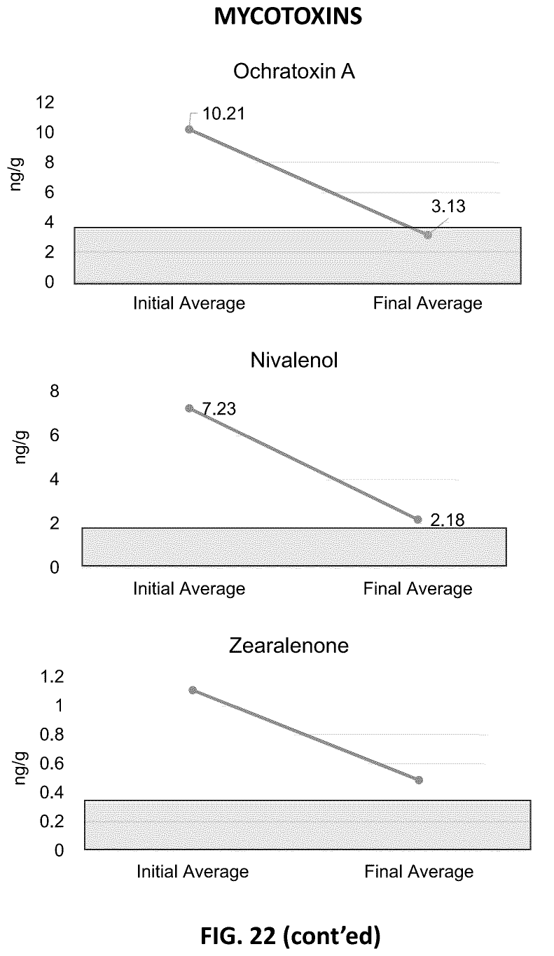

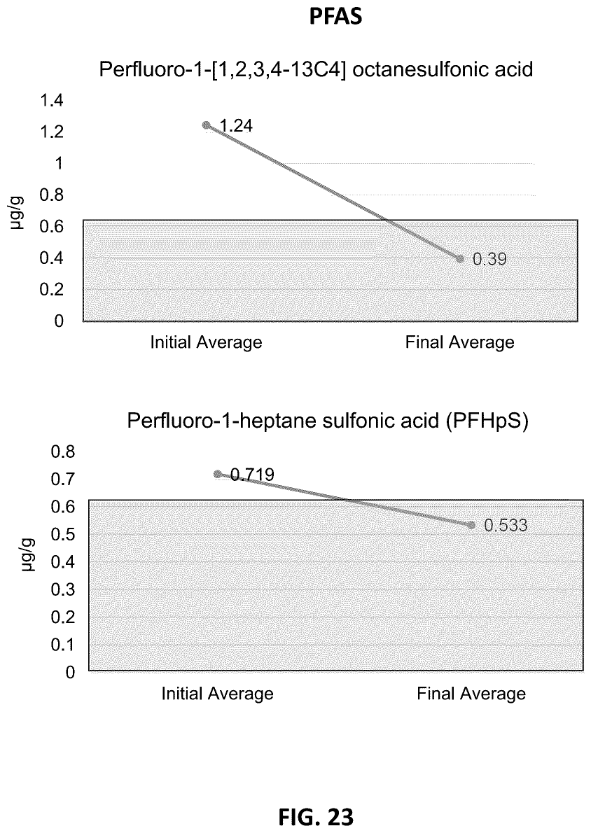

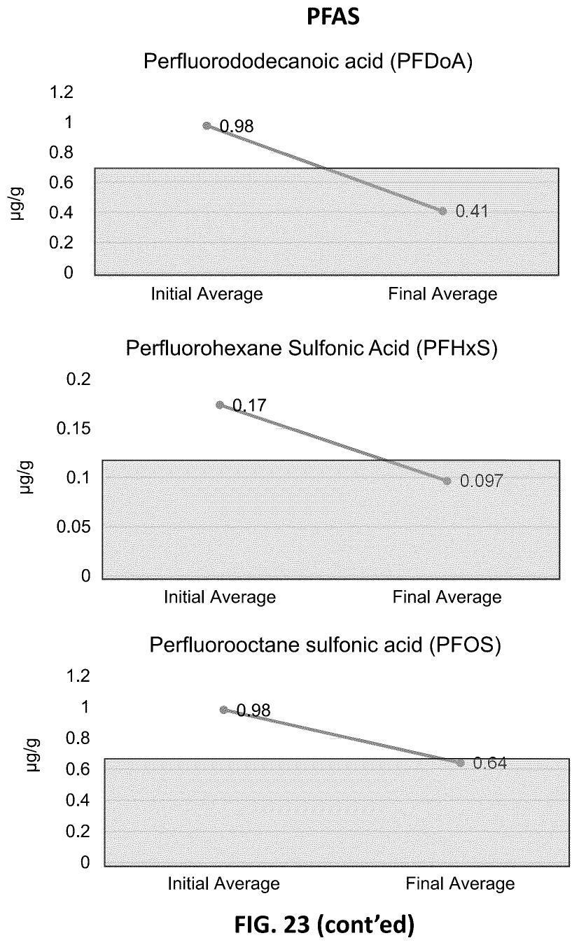

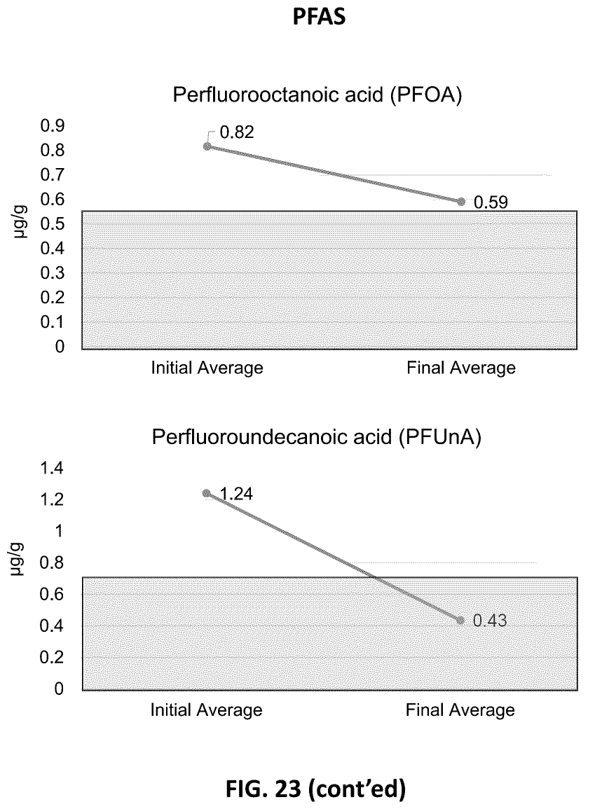

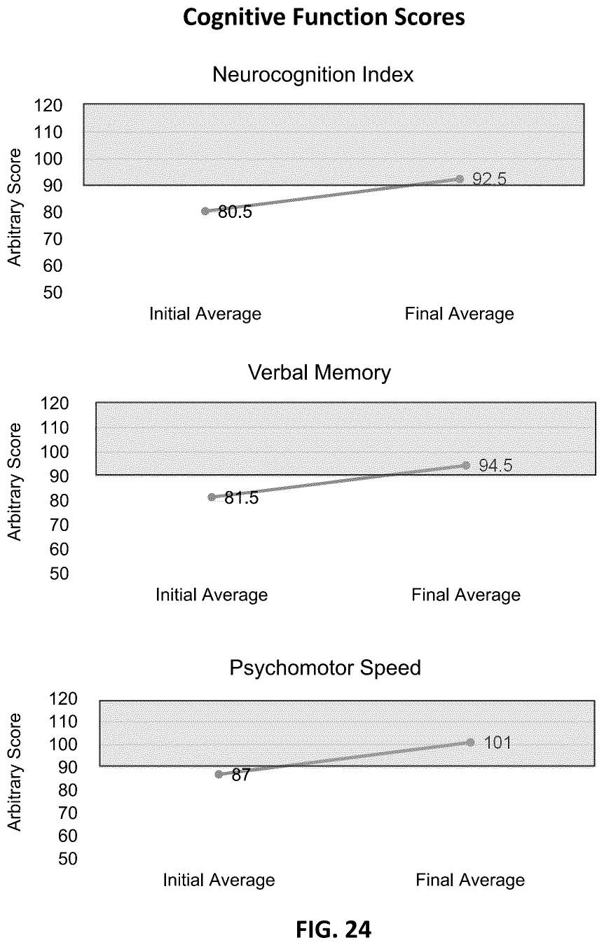

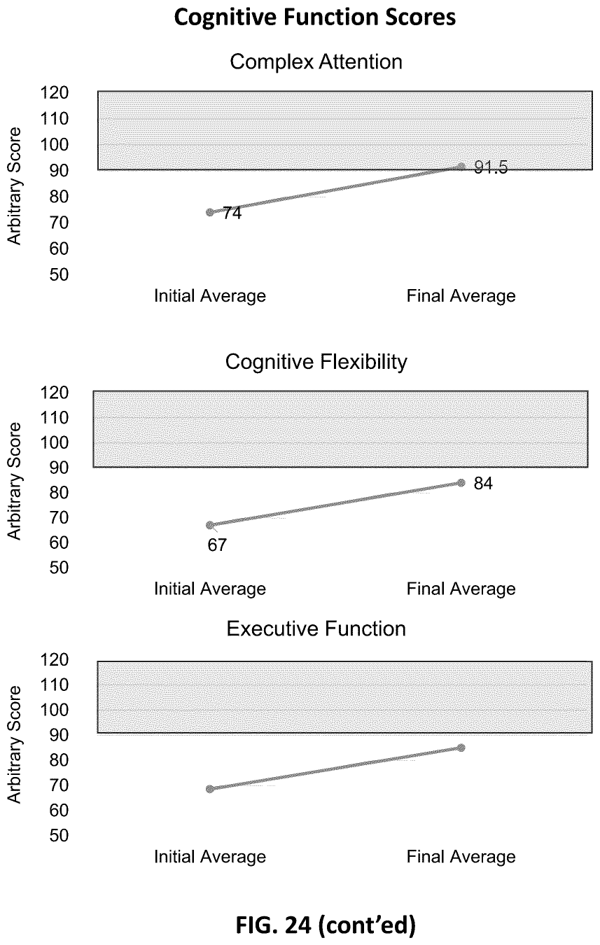

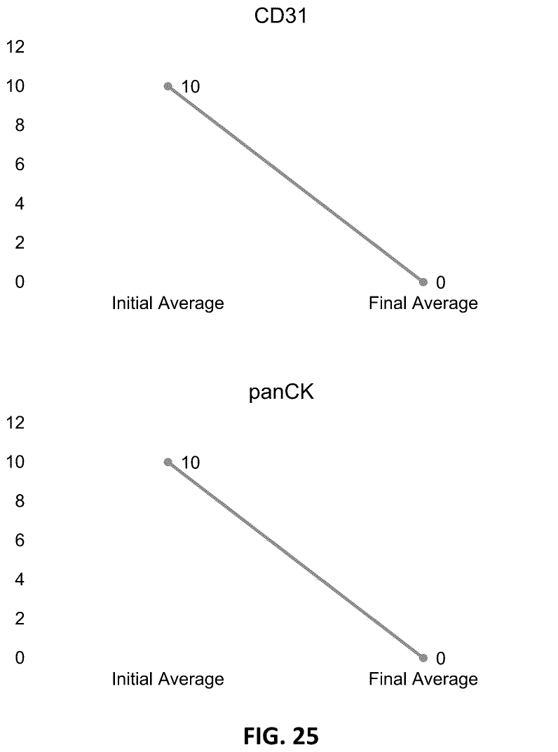

Table 11 shows the average data of all patients receiving combined TPE, brain TPE, or cancer TPE treatment. The overall trend of the tested biomarkers (for immune system, inflammation, oxidative stress, and cancer) showed improvement towards the healthy range (FIG. 14 through FIG. 16 and FIG. 25). The toxin levels were also reduced towards, and in some cases, to the normal range (FIG. 17 through FIG. 24). In most cases, these changes in biomarkers and toxin levels were better with the combined TPE, brain TPE, and cancer TPE treatments compared to the control TPE treatment (Table 11), indicating that the supplementations (oral and IV compositions administrations) and other components (such as stem cells, anti-inflammatory and anti-fungal medications) boosted the effects of the TPE.

TABLE 11
Percentage change (% change) at 6 weeks (6 w) after control TPE
treatment (TPE only) and TPE treatments that incorporating the
combined TPE treatment (combined TPE treatment, brain TPE treatment,
and cancer TPE treatment) compared to before the treatments.

| | | Combined TPE, | | | |
|---|---|---|---|---|---|
| | | Brain TPE, and | | | |
| | | Cancer TPE | | Control TPE | |
| | | Percentage | | Percentage | |
| | | difference | | difference | |
| | | in average | | in average | |
| | | before and | | before and | |
| | Biomarkers/Toxins/Function | after | | after | |
| Category | Tests | treatment | Count | treatment | Count |
| Immune | CD3 | 25.25 | 7 | 2.25 | 3 |
| System | | | | | |
| | CD4 | 26.80 | 8 | 6.56 | 4 |
| | CD8 | 13.70 | 13 | −2.13 | 4 |
| | CD19 | 14.42 | 19 | 2.93 | 5 |
| | CD56 | 65.68 | 10 | −1.37 | 2 |
| Inflammation | Uric Acid (UA) | −14.62 | 6 | −5.43 | 3 |
| | hs-C Reactive Protein | −51.01 | 5 | −18.29 | 4 |
| | Interleukin-6 | −15.32 | 1 | 31.15 | 3 |
| | Interleukin-8 | −86.18 | 2 | −2.22 | 3 |
| | Tumor Necrosis Factor alpha | −16.38 | 1 | 0.63 | 2 |
| Longevity | β-galactosidase | 27.99 | 8 | −30.14 | 3 |
| Oxidative | Myeloperoxidase | −53.08 | 8 | −28.88 | 2 |
| Stress | | | | | |
| Heavy Metals | Antimony | −94.44 | 3 | −73.21 | 2 |
| | Aluminum | −83.15 | 4 | | |
| | Arsenic | −74.63 | 7 | −65.68 | 2 |
| | Cadmium | −56.58 | 9 | −39.47 | 1 |
| | Cesium | −43.57 | 6 | | |
| | Beryllium | −77.22 | 3 | | |
| | Bismuth | −87.44 | 4 | | |
| | Nickel | −71.38 | 6 | | |
| | Tellurium | −83.80 | 4 | −80.95 | 1 |
| | Barium | −36.06 | 5 | −46.17 | 1 |
| | Gadolinium | −66.06 | 3 | −31.97 | 1 |
| | Tungsten | −82.69 | 6 | −60.00 | 1 |
| | Thallium | −61.50 | 9 | | |
| | Thorium | −100.00 | 3 | | |
| | Tin | −92.11 | 4 | | |
| | Mercury | −79.83 | 11 | −60.25 | 1 |
| | Lead | −69.03 | 9 | −53.40 | 2 |
| | Uranium | −81.82 | 1 | | |
| Environmental | Perchlorate | −72.29 | 3 | | |
| Toxins | | | | | |
| Volatile | N-acetyl-S-(2- | −76.70 | 2 | −55.67 | 1 |
| Compounds | carbamoylethyl)-cysteine | | | | |
| | (NAE) | | | | |
| | Tiglylglycine (TG) | −23.69 | 5 | −48.15 | 1 |
| | 4-Methylhippuric Acid | −95.56 | 1 | | |
| | (4MHA) | | | | |
| | 2-Hydroxyisobutyric Acid | −69.64 | 6 | | |
| | (2HIB) | | | | |
| | N-Acetyl Propyl Cysteine | −78.74 | 2 | | |
| | (NAPR) | | | | |
| | Phenylglyoxylic Acid (PGO) | −80.88 | 1 | | |
| Herbicides | Atrazine | −88.24 | 1 | −83.33 | 1 |
| | Glyphosate | −69.03 | 9 | −24.79 | 2 |
| | 2,4-Dichlorophenoxyacetic | −76.69 | 2 | | |
| | Acid (2,4-D) | | | | |
| Paraben | Butylparaben | −39.18 | 4 | | |
| | Propylparaben | −51.56 | 1 | | |
| Pesticides | 2,2-bis(4-Chlorophenyl) acetic | −49.21 | 2 | | |
| | acid (DDA) | | | | |
| | Dimethyldithiophosphate | −62.19 | 7 | −51.32 | 1 |
| | (DMDTP) | | | | |
| | Dimethylphosphate (DMP) | −67.45 | 9 | −67.73 | 1 |
| | Dimethylthiophosphate | −32.96 | 4 | −40.30 | 1 |
| | (DMTP) | | | | |
| | Diethyldithiophosphate | −61.04 | 3 | | |
| | (DEDTP) | | | | |
| Phenols | Bisphenol A (BPA) | −44.18 | 11 | −18.83 | 2 |
| | Triclosan | −37.49 | 6 | | |
| | 4-Nonylphenol | −60.48 | 2 | −53.64 | 1 |
| Phthalates | mono-(2-ethyl-5-oxohexyl) | −61.70 | 7 | | |
| | phthalate (MEOHP) | | | | |
| | mono-2-ethylhexyl phthalate | −88.29 | 2 | | |
| | (MEHP) | | | | |
| Mycotoxins | Aflatoxin B1 | −38.76 | 3 | | |
| | Aflatoxin B2 | −64.93 | 1 | | |
| | Aflatoxin G1 + B93: C94 | −33.69 | 4 | | |
| | Aflatoxin G2 | −55.52 | 2 | | |
| | Aflatoxin M1 | −65.31 | 1 | | |
| | Chaetoglobosin A | −71.28 | 1 | | |
| | Enniatin B1 | −79.41 | 1 | | |
| | Dihydrocitrinone | −65.40 | 2 | −74.77 | 1 |
| | Citrinin | −54.48 | 5 | −45.74 | 1 |
| | Nivalenol | −65.01 | 3 | −8.76 | 1 |
| | Fumonisins B1 | −49.16 | 7 | −26.26 | 1 |
| | Fumonisins B2 | −25.20 | 4 | −1.20 | 1 |
| | Fumonisins B3 | −69.88 | 6 | | |
| | Gliotoxin | −21.07 | 5 | −32.48 | 2 |
| | Ochratoxin A | −69.26 | 7 | −35.00 | 1 |
| | Zearalenone | −49.90 | 5 | −21.34 | 1 |
| | Patulin | −3.73 | 2 | | |
| | Deoxynivalenol | −15.78 | 3 | | |
| | Diacetoxyscirpenol | −43.22 | 6 | | |
| | Roridin A | −45.98 | 2 | | |
| | Roridin E | −51.19 | 2 | | |
| | Roridin L2 | −37.96 | 3 | | |
| | Satratoxin G | 0.00 | 1 | | |
| | T-2 toxin | −9.09 | 1 | | |
| | Verrucarin A | −44.05 | 1 | | |
| | Verrucarin J | −68.60 | 3 | | |
| PFAS | GenX/HPFO-DA | −16.51 | 5 | | |
| | Perfluorooctane sulfonic acid | −34.83 | 1 | | |
| | (PFOS) | | | | |
| | Perfluorooctanoic acid (PFOA) | −29.28 | 4 | | |
| | Perfluorohexane Sulfonic Acid | −42.71 | 2 | | |
| | (PFHxS) | | | | |
| | Perfluorododecanoic acid | −48.50 | 4 | −21.60 | 1 |
| | (PFDoA) | | | | |
| | Perfluorohexanoic acid | −26.35 | 4 | −15.79% | 1 |
| | (PFHxA) | | | | |
| | Perfluoropentanoic acid | −40.89 | 3 | −23.53% | 1 |
| | (PFPeA) | | | | |
| | Perfluoro-1-[1,2,3,4-13C4] | −63.63 | 2 | | |
| | octanesulfonic acid | | | | |
| | Perfluoro-1-heptane sulfonic | −25.87 | 1 | | |
| | acid (PFHpS) | | | | |
| | Perfluoroundecanoic acid | −70.42 | 2 | | |
| | (PFUnA) | | | | |
| Cancer | CD31 | −100.00 | 3 | −100.00 | 1 |
| markers | | | | | |
| | panCK | −100.00 | 3 | −100.00 | 1 |
| Cognitive | Neurocognition Index (NCI) | 15.15 | 2 | | |
| function | Std Score | | | | |
| | Composite Memory Std Score | 7.35 | 2 | 6.90 | 1 |
| | Verbal Memory Std Score | 16.11 | 2 | | |
| | Visual Memory Std Score | 11.54 | 1 | 3.75 | 1 |
| | Psychomotor Speed Std Score | 16.09 | 1 | | |
| | Reaction Time Std Score | 6.22 | 2 | 0.00 | 1 |
| | Complex Attention Std Score | 25.98 | 2 | | |
| | Cognitive Flexibility Std Score | 27.19 | 2 | | |
| | Executive Function Std Score | 26.03 | 2 | | |
| | Simple Attention Std Score | 13.41 | 1 | 0.00 | 1 |

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings of the disclosure, and the appended claims. The mere fact that certain measures or features are recited in mutually different dependent claims does not indicate that the combination of these measures or features cannot be used. Any reference signs in the claims should not be construed as limiting the scope.

1: A method of removing toxins in a patient as a result of chronic exposure to the toxins, comprising:
(a) performing one or more therapeutic plasma exchange (TPE) sessions on the patient; and
(b) administering an oral composition to the patient.

2: The method of claim 1, wherein each TPE session is performed with 0.8 to 1.5 estimated plasma volume (EPV), 5% to 10% albumin, and/or an anticoagulant.

3-4. (canceled)

5: The method of claim 2, wherein the anticoagulant is citrate or heparin.

6: The method of claim 1 further comprising administering a metal chelator to the patient during step (a).

7: The method of claim 6, wherein the metal chelator is dimercaptosuccinic acid (DMSA), sodium 2,3 dimercaptopropanel sulphonate, ethylenediaminetetraacetic acid (EDTA), sodium calcium edetate, D-penicillamine, or N-acetyl-DL-penicillamine.

8: The method of claim 1, wherein the oral composition is administered to the patient daily after the first TPE session.

9: The method of claim 1, wherein the patient has atherosclerosis.

10: The method of claim 9, wherein the method is capable of reducing the level or slowing the progression of atherosclerosis in the patient.

11: The method of claim 9 further comprising administering at least one cholesterol-reducing agent to the patient after completing the first TPE session.

12: The method of claim 11, wherein the at least one cholesterol-reducing agent has not been administered to the patient prior to commencing the first TPE session.

13: The method of claim 11, wherein the at least one cholesterol-reducing agent is administered to the patient after completing the first TPE session.

14: The method of claim 1, wherein the patient has declined cognitive function.

15: The method of claim 14, wherein the method is capable of improving cognitive function or immune function, or treating cancer in the patient.

16-17. (canceled)

18: The method of claim 15 further comprising administering low dose naltrexone to the patient.

19: The method of claim 18 further comprising administering one or more neuropeptides to the patient prior to step (a).

20: The method of claim 19, wherein the neuropeptide is adrenocorticotropin, a tetrapeptide having the amino acid sequence of AEDG (SEQ ID NO: 1), a heptapeptide having the amino acid sequence of TKPRPGP (SEQ ID NO: 2), a tripeptide having the amino acid sequence of EDR, a tripeptide having the amino acid sequence of KED, fibroblast growth loop peptide (FGL), brain-derived neurotrophic factor (BDNF), glial cell line-derived neurotrophic factor (GDNF), nerve growth factor (NGF), ciliary neurotrophic factor (CNTF), hypocretin-1, low molecular peptides with a molecular weight up to 5000 Da isolated from the brain tissues of calves, or low molecular peptides with a molecular weight up to 10000 Da isolated from the vascular (aorta) tissue of calves, or a combination thereof.

21: The method of claim 15, wherein step (a) further comprises administering exosomes, mesenchymal stem cells (MSCs), or a combination thereof to the patient after completing at least one TPE session but no more than two consecutive TPE sessions.

22: The method of claim 21, wherein the MSCs and/or exosomes are derived from umbilical cord.

23. (canceled)

24: The method of claim 21, wherein the exosomes, mesenchymal stem cells (MSCs), or a combination thereof are administered to the patient after completing at least one TPE session but no more than two consecutive TPE sessions.

25: The method of claim 17 further comprising administering at least one anti-inflammatory compound, one anti-fungal compound, or a combination thereof to the patient.

26: The method of claim 25 further comprising administering thymosin al, met enkephalin, methylene blue, nystatin, diflucan, colloidal silver, mebendazole, or a combination thereof to the patient.

27: The method of claim 1, wherein step (a) further comprises administering immunoglobulin to the patient after each TPE session.

28: The method of claim 1, wherein between 3 and 8 TPE sessions are performed and each TPE session is performed between 1 and 6 weeks after the preceding TPE session.

29: The method of claim 1 further comprising removing plasma from the patient at least once every 1 to 20 weeks after the last TPE session.

30. (canceled)

31: The method of claim 1, wherein the level of one or more toxins is reduced in the patient.

32: The method of claim 31, wherein the one or more toxins is a heavy metal, herbicide, pesticide, phenol, phthalate, volatile organic compound, mycotoxin, per- and/or polyfluorinated substance (PFAS), microplastic, or a combination thereof.

33: The method of claim 1, wherein the method is capable of increasing the level of one or more biomarkers indicating an improvement in immune response in the patient.

34. (canceled)

35: The method of claim 1, wherein the method is capable of reducing the level of one or more biomarkers indicating a suppression of inflammation in the patient.

36. (canceled)

37: The method of claim 1, wherein the method is capable of increasing the level of one or more biomarkers associated with longevity, oxidative stress, and/or cellular senescence in the patient.

38-42. (canceled)

43: The method of claim 1, wherein each TPE session of step (a) further comprises:
(i) administering an intravenous (IV) composition to the patient after performing TPE on the patient; and
(ii) administering a glutathione solution to the patient.

44: The method of claim 43, wherein the IV composition comprises:
(a) at least one mineral;
(b) at least one vitamin; and
(c) at least one amino acid.

45: The method of claim 44, wherein the IV composition comprises the mineral magnesium; the vitamins ascorbic acid, dexpanthenol, hydroxocobalamin, niacin, pyridoxine, riboflavin, and thiamine; and the amino acids carnitine and taurine.

46: The method of claim 44, wherein:
(a) the at least one mineral is one or more of calcium, magnesium, molybdenum, potassium, selenium, and/or zinc;
(b) the at least one vitamin is one or more of ascorbic acid, dexpanthenol, hydroxocobalamin, niacin, pyridoxine, riboflavin, and/or thiamine; and/or
(c) the at least one amino acid is carnitine and/or taurine.

47-48. (canceled)

49: The method of claim 1, wherein the oral composition comprises:
(a) at least one vitamin;
(b) at least one mineral;
(c) at least one anti-inflammatory agent;
(d) at least one antioxidant;
(e) at least one amino acid;
(f) at least one saccharide; and
(g) at least one herb.

50: The method of claim 49, wherein the oral composition further comprises at least one protein.

51: The method of claim 49, wherein the oral composition comprises the vitamins ascorbic acid, cobalamin, folate, nicotinamide riboside chloride, pantothenic acid, pyridoxine, riboflavin, and vitamin E; the minerals calcium and magnesium; the anti-inflammatory agents baicalin, Noni fruit extract, oxindole alkaloid, omega-3, superoxide dismutase, resveratrol, and pterostilbene; the antioxidants coenzyme Q10, glutathione, epigallocatechin gallate, cysteine, punicalagin, quercetin, rosemary extract, turmeric extract, silymarin, shilajit extract, glucoraphanin, polyphenols, Schisandra berry extract, grape seed extract, micro pyrroloquinoline quinone, alpha lipoic acid, boswellin, and dandelion root extract; the amino acids arginine, glutamine, proline, and tyrosine; the saccharides larch arabinogalactan, ribose, and beta glucan; and the herbs gingerol and bitter melon extract.

52: The method of claim 50, wherein the protein is rice protein.

53: The method of claim 49, wherein the oral composition further comprises at least one other component.

54: The method of claim 53, wherein the at least one other component is vinpocentine or Panax ginseng.

55: The method of claim 49, wherein:
(a) the at least one vitamin is one or more of biotin, cobalamin, folate, pantothenic acid, pyridoxine, riboflavin, thiamine, vitamin A, vitamin B3, vitamin C, vitamin D3, and/or vitamin E;
(b) the at least one mineral is one or more of calcium, chromium, copper, iodine, magnesium, manganese, molybdenum, and/or zinc;
(c) the at least one anti-inflammatory agent is one or more of baicalin, bromelain, Noni fruit extract, omega-3, oxindole alkaloid, pterostilbene, resveratrol, and/or superoxide dismutase;
(d) the at least one antioxidant is one or more of alpha lipoic acid, astaxanthin, Boswellia serrata extract, coenzyme Q10, Curcuma longa extract, cysteine, dandelion root extract, epigallocatechin gallate, Ginkgo biloba, glucoraphanin, glutathione, grape seed extract, hops extract, micro pyrroloquinoline quinone, polyphenols, punicalagin, quercetin, rosemary extract, schisandra berry extract, shilajit extract, and/or silymarin;
(e) the at least one amino acid is one or more of alanine, arginine, glutamine, glycine, lysine, methionine, ornithine, phosphatidylserine, proline, tyrosine, taurine, theanine, and/or trimethyl glycine;
(f) the at least one saccharide is one or more of larch arabinogalactan, beta glucan, and/or ribose; and/or
(g) the at least one herb is Bacopa monnieri, Berberis aristata, bitter melon extract, Citrus bergamia, gingerol, Hericium erinaceus, and/or Withania somnifera.

56-61. (canceled)

62: An IV composition for detoxification in a patient, comprising one or more of:
(a) at least one mineral;
(b) at least one vitamin; and
(c) at least one amino acid.

63: An oral composition for detoxification in a patient, comprising one or more of:
(a) at least one vitamin;
(b) at least one mineral;
(c) at least one anti-inflammatory agent;
(d) at least one antioxidant;
(e) at least one amino acid;
(f) at least one saccharide; and
(g) at least one herb.
